(19) United States
(12) Patent Application Publication
(10) Pub. No.: US 2008/0220416 A1
(43) Pub. Date: Sep. 11, 2008

(54) Compositions for Inhibiting Cell Growth and Inducing Apoptosis in Cancer Cells and Methods of Use Thereof

(75) Inventors: Lucio Miele, Batavia, IL (US); Brian J. Nickoloff, Burr Ridge, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: THE BOARD OF TRUSTEES OF THE UNIVERSITY OF ILLINOI, Urbana, IL (US)

(21) Appl. No.: 11/569,327

(22) PCT Filed: May 20, 2005
(86) PCT No.: PCT/US05/17768

§ 371 Date: Apr. 11, 2008

Related U.S. Application Data
(60) Provisional application No. 60/572,819, filed on May 20, 2004.
(60) Provisional application No. 60/585,317, filed on Jul. 2, 2004.

(51) Int. Cl. C12Q 1/68

(52) U.S. Cl. 435/6

(57) ABSTRACT

This invention provides compounds, methods and pharmaceutical compositions for inhibiting cancer cell growth and inducing apoptosis in cancer cells, particularly cells resistant to conventional chemotherapeutic drug treatment.

### CROSS-REFERENCE TO RELATED APPLICATIONS

This application claims priority from U.S. provisional patent application Ser. No. 60/572,819 filed May 20, 2004 and Ser. No. 60/585,317 filed Jul. 2, 2004, the disclosure of each of which is incorporated by reference herein.

### STATEMENT REGARDING FEDERALLY SPONSORED RESEARCH

This research was sponsored in part by the United States Government, Department of Health and Human Services, under grants from the National Institutes of Health identifies by grant nos. PO159327, RO1CA84065. The government has certain rights in this invention

### BACKGROUND OF THE INVENTION

Cellular growth of normal tissue is maintained in homeostasis. This balance is determined by cellular proliferation and renewal on one hand, and cell death on the other. Neoplasia can result from aberrant regulation of this homeostasis, through inter alia somatic genetic abnormalities that cause cancer initiation and progression. It has long been known that cancerous tumors are created by the unregulated growth of undifferentiated tumor cells. Hence, scientists have long sought therapies that would prevent tumor cells from growing.

Conventional treatments for cancer using radiation or chemotherapeutic drugs target rapidly proliferating cells and induce such cells to undergo apoptosis. Apoptosis, or programmed cell death, is a normal cellular process that is critical for differentiation during embryogenesis and regulation of cell numbers. It can also be induced in neoplastic cells, so that they self-destruct. A growing body of evidence suggests that the intracellular “death program” activated during apoptosis is similar in different cell types and has been conserved during evolution.

Apoptosis involves two essential steps. The first, or “decision” step of apoptosis involves expression (or lack thereof) of members of the Bcl-2 family of proteins, which consists of different anti- and pro-apoptotic members. The other, or “execution” step, in contrast, is mediated by the activation of caspases, cysteine proteases that induce cell death via proteolytic cleavage of substrates vital for cellular homeostasis. In this arrangement of the steps of apoptosis Bcl-2-related proteins act “upstream” from caspases in the cell death pathway.

Extensive efforts have been directed toward developing chemotherapeutic drugs suitable for treating cancer. Most chemotherapeutic agents exhibit some degree of toxicity toward normal cells at therapeutic doses, causing undesired side effects that may be dose limiting, thereby reducing the usefulness of the drug. Furthermore, these traditional methods of treatment are not successful in treating many types of cancers, particularly those that are resistant to apoptotic stimuli. Because most genotoxic agents act primarily through p53-dependent induction of apoptosis, resistance to apoptotic stimuli is often attributed to mutations in a p53 gene.

Typically, individual drugs used alone are often ineffective in treating cancer. Advances in chemotherapy have been made largely through empirical identification of useful combinations of drugs through a process of trial and error. Although progress has been made in discovering and developing new drugs or drug combinations that are at least partially effective against certain types of cancer, there is an ongoing need in the art for improved methods of treating cancer and of discovering useful combinations of drugs or other agents to treat cancer.

One approach to tumor therapy is to identify agents that act to initiate programmed cell death, or apoptosis. Another approach to tumor therapy is to identify agents that act to inhibit cell growth. In view of the high incidence of cancers that are refractory to current treatment methods, there is also an ongoing need for new methods of inducing apoptosis in cancer cells and/or new methods for inhibiting cell growth in cancer cells, for new methods of identifying molecules capable of inducing apoptosis and/or inhibiting cell growth in cancer cells, and for new compounds and pharmaceutical compositions that induce apoptosis in cancer cells and/or inhibit cell growth in cancer cells.

### SUMMARY OF THE INVENTION

The invention provides methods for inducing apoptosis in cancer cells comprising the step of contacting cancer cells with combinations of a gamma secretase inhibitor, a proteasome inhibitor, a tumoricidal agent or a compound that inhibits Notch-1 gene expression or protein activity in amounts and for periods of time sufficient to induce apoptosis in said cancer cells.

The invention further provides methods of inhibiting cell growth in cancer cells comprising the step of contacting cancer cells with combinations of a gamma secretase inhibitor, a proteasome inhibitor, a tumoricidal agent or a compound that inhibits Notch-1 gene expression or protein activity in amounts and for periods of time sufficient to inhibit cell growth in said cancer cells.

The invention also provides methods for screening compounds for NOXA gene expression-inducing ability in cancer cells comprising:
(a) contacting cancer cells with a culture media in the presence and absence of a test compound;
(b) assaying the cells of step (a) for NOXA gene expression;
(c) comparing NOXA gene expression assayed in step (b) from cells contacted with culture media in the presence of the test compound with NOXA gene expression from cells contacted with culture media in the absence of the test compound; and
(d) identifying a compound that induces NOXA gene expression when NOXA gene expression is higher in cells contacted with a culture media in the presence of the test compound than in cells contacted with a culture media in the absence of the test compound.

In addition, the invention provides methods for screening a test compound for Notch-1 gene expression- or activity-inhibiting ability in cancer cells, the method comprising the steps of:
(a) contacting cancer cells with a culture media in the presence and absence of a test compound;
(b) assaying the cells of step (a) for Notch-1 gene expression or activity;
(c) comparing Notch-1 gene expression or activity assayed in step (b) from cells contacted with culture media in the presence of the test compound with Notch-1 gene expression or activity from cells contacted with culture media in the absence of the test compound; and
(d) identifying a compound that inhibits Notch-1 gene expression or activity when Notch-1 gene expression or activity is lower in cells contacted with culture media in the presence of the test compound than in cells contacted with culture media in the absence of the test compound.

The invention further provides methods for inducing apoptosis or inhibiting cell growth in cancer cells comprising the step of contacting the cancer cells with an apoptosis-inducing effective amount or a growth-inhibiting effective amount of N-benzyloxycarbonyl-leucyl-leucyl-norleucinal.

The invention also provides methods for inducing apoptosis in melanoma cells, myeloma cells, prostate cancer cells, osteosarcoma cells, breast cancer cells or cervical cancer cells comprising the step of contacting the cancer cells with an apoptosis inducing effective amount of a combination of a gamma secretase inhibitor, a proteasome inhibitor, a tumoricidal agent or a compound that inhibits Notch-1 gene expression or protein activity.

The invention also provides methods for inhibiting cell growth in melanoma cells, myeloma cells, prostate cancer cells, osteosarcoma cells, breast cancer or cervical cancer cells comprising the step of contacting the cancer cells with a growth inhibiting-effective amount of a combination of a gamma secretase inhibitor, a proteasome inhibitor, a tumoricidal agent or a compound that inhibits Notch-1 gene expression or protein activity.

In addition, the invention provides pharmaceutical compositions that induce apoptosis in cancer cells comprising a combination of a gamma secretase inhibitor, a proteasome inhibitor, a tumoricidal agent or a compound that inhibits Notch-1 gene expression or protein activity, and a pharmaceutically-acceptable carrier, excipient, diluent or adjuvant.

In addition, the invention provides pharmaceutical compositions that inhibit cell growth in cancer cells comprising a combination of a proteasome inhibitor, gamma secretase inhibitor, a tumoricidal agent or a compound that inhibits Notch-1 gene expression or protein activity and a pharmaceutically-acceptable carrier, excipient, diluent or adjuvant.

The invention also provides methods of treating an animal that has melanoma, prostate cancer, multiple myeloma, osteosarcoma, breast cancer or cervical cancer comprising the step of administering a therapeutically-effective amount of the pharmaceutical compositions of the invention.

The methods of the invention are advantageously practiced using, and the inventive methods provided use, compounds that induce apoptosis in a cancer cell having the formula:
Z-(AA)ₙ-Y

wherein
each AA is independently a hydrophobic L- or D-amino acid, a hydrophobic non-naturally-occurring amino acid, a sulfonamide or a benzodiazepine, wherein at least one amino acid can be different from at least one other amino acid;
Z is an acyl group or is absent;
Y is an aldehyde group, a boronate group, an α-keto acid, an α-keto ester, an α-keto amide, an epoxyketone, a vinyl sulfone, an α-keto heterocycle or is absent; and
n is an integer from 3 to 5 when AA is an amino acid and n is 1 when AA is a sulfonamide or a benzodiazepine.

Specific preferred embodiments of the invention will become evident from the following more detailed description of certain preferred embodiments and the claims.

### BRIEF DESCRIPTION OF THE DRAWINGS

FIG. 1 shows selective induction of apoptosis by treatment of melanoma cells in vitro and in vivo with N-benzyloxycarbonyl-leucyl-leucyl-norleucinal (z-Leu-Leu-Nle-CHO). FIG. 1A is a graph showing the apoptotic effect of 1-10 μM z-Leu-Leu-Nle-CHO in melanoma cells carrying both wild-type and mutated p53 but not melanocytes. FIG. 1B shows a Western blot for Apaf-1 expression levels in melanoma cells and melanocytes that have not been treated with a gamma secretase inhibitor. Actin was used as a loading control. FIG. 1C shows results from FACS analysis of melanoma cells and melanocytes following treatment with z-Leu-Leu-Nle-CHO, indicating that z-Leu-Leu-Nle-CHO induces apoptosis in melanoma cells, but only a G2/M growth arrest in melanocytes. FIG. 1D is a hematoxylin and eosin (H&E) stained frozen section of highly aggressive human cutaneous melanoma tumor xenograft (C8161 cells) treated with z-Leu-Leu-Nle-CHO (1 mM), for a week. FIG. 1E is photo of highly aggressive human cutaneous melanoma tumor xenograft (C8161 cells) showing a high level of apoptosis observed by immunofluorescent TUNEL assay following 1 week treatment with z-Leu-Leu-Nle-CHO. FIG. 1F is photo of highly aggressive human cutaneous melanoma tumor xenograft (C8161 cells) showing a lack of apoptosis observed by immunofluorescent TUNEL assay following 1 week treatment with DMSO carrier.

FIG. 1G is a graph of the average tumor size prior to treatment (week 1) and 1 week after treatment (week 2) with z-Leu-Leu-Nle-CHO (“GSI”) or DMSO. z-Leu-Leu-Nle-CHO significantly reduced the in average tumor size after 1 week of treatment (p=0.046) compared to DMSO (n=11).

FIG. 2 illustrates z-Leu-Leu-Nle-CHO induction of pro-apoptotic proteins including p53-independent induction of NOXA in melanoma cell, prostate cancer cells and osteosarcoma cells. FIG. 2A is a Western blot showing the profile of pro-survival and pro-apoptotic proteins in RJ002L melanoma cells following z-Leu-Leu-Nle-CHO exposure (10 μM; left side) compared to medium alone (right side). NOXA expression is induced upon z-Leu-Leu-Nle-CHO exposure.

FIG. 2B is a Western blot showing that z-Leu-Leu-Nle-CHO triggers release of cytochrome C and SMAC from the mitochondria of RJ002L cells. FIG. 2C is a Western blot showing induction of NOXA by z-Leu-Leu-Nle-CHO in melanoma cell lines with either mutant p53 (C8161, MUM2B and SK-MeI-28) or wild-type p53 (SK-MeI-100), indicating that induction of NOXA by z-Leu-Leu-Nle-CHO is p53-independent. FIG. 2D is a Western blot of RJ002L melanoma cells treated with control or p53-specific siRNA, in the presence or absence of z-Leu-Leu-Nle-CHO (“GSI”). The blot shows that reduction of p53 levels in RJ002L cells using p53 siRNA leads to a reduction of z-Leu-Leu-Nle-CHO-induced GADD45 and MDM2 levels, but not NOXA levels. FIG. 2E is a Western blot of two different non-melanoma cell lines, prostate cancer (PC-3) and osteosarcoma (SAOS-2) cell lines, treated with z-Leu-Leu-Nle-CHO. The blot shows induction of NOXA by z-Leu-Leu-Nle-CHO non-melanoma cell lines even though the cell lines are deficient in p53.

FIG. 3 shows that inhibition of NOXA expression with antisense oligonucleotides reduces induction of apoptosis by z-Leu-Leu-Nle-CHO. FIG. 3A is a Western blot showing that a NOXA-specific antisense oligonucleotide (“ASO”), but not a control oligonucleotide (“Control Oligo”), reduced z-Leu-Leu-Nle-CHO-induced NOXA levels, but not Bim or Bak levels. Actin was used as a loading control. FIG. 3B is a graph showing the effect of z-Leu-Leu-Nle-CHO in melanoma cells treated with control oligonucleotide (“Control Oligo” or “CO”) or NOXA-specific ASO on cell cycle population distribution. NOXA ASO but not CO reduced the z-Leu-Leu-Nle-CHO-mediated induction of apoptosis (sub-G₀ DNA content) accompanied by increased G2/M growth arrest in RJ002L cells. FIG. 3C is a graph showing the effect of z-Leu-Leu-Nle-CHO (“GSI”) on cell cycle population distribution in three different melanoma cell lines (RJ002L, C8161, MUM2B) treated with control oligonucleotide (“CO”) or NOXA-specific ASO (“NOXA ASO”). NOXA ASO, but not CO reduced z-Leu-Leu-Nle-CHO-mediated induction of apoptosis (sub-G₀ DNA content). (p<0.01).

FIG. 4 shows induction of apoptosis by proteasome inhibitors in melanoma cells but not normal melanocytes. FIG. 4A is a graph showing the percentage of dead cells in populations of normal melanocytes and melanoma cells treated with various concentrations of the proteasome inhibitor MG-132. The graph shows a dose-dependent induction of apoptosis in melanoma cells, but not normal melanocytes, upon exposure to MG-132. The melanoma cell lines undergo significantly enhanced apoptotic responses to increasing concentrations of MG-132, irrespective of their p53 status. FIG. 4B is a graph showing the percentage of dead cells in populations of normal melanocytes and melanoma cells treated with various concentrations of the proteasome inhibitor lactacystin. The graph shows a dose-dependent induction of apoptosis in melanoma cells, but not normal melanocytes, by lactacystin. The melanoma cell lines undergo significantly enhanced apoptotic responses to increasing concentrations of lactacystin, irrespective of their p53 status. FIG. 4C are phase contrast microscopy images of RJ002L melanoma cells before, and after exposure (24 hours), to bortezomib (“Velcade”) (at concentrations from 0.001 to 10 μM). No apoptotic response is noted in the concentration of 0.01 μM (2% of cells with sub-G₀ DNA content by FACS analysis), but increased concentrations of bortezomib triggered an increase in apoptotic cells. Melanoma cells exposed to concentrations of bortezomib ≧1 μM appeared rounded-up, with membrane blebbing and detachment from the dish. The cells were exposed to the following concentrations of bortezomib: top left, 0 μM; top middle, 0.001 μM; top right, 0.01 μM; bottom left, 0.1 μM; bottom middle, 1 μM; bottom right, 10 μM.

FIG. 5 shows induction of apoptosis by proteasome inhibitors in melanoma cells, but not normal melanocytes. FIG. 5A is a graph showing the results of a kinetic analysis for induction of apoptosis in RJ002L melanoma cells using the proteasome inhibitor bortezomib. Bortezomib causes minimal changes in viability of the culture over the initial 6 hrs of exposure, but prominent apoptosis was detected at 18- and 24-hr time points. FIG. 5B is a graph showing the percentage of dead cells in populations of normal melanocytes and melanoma cells treated with various concentrations of the proteasome inhibitor bortezomib. Bortezomib failed to trigger an apoptotic response in proliferating melanocytes (FIG. 5B, left side panel). In contrast, bortezomib triggered a dose-dependant increase in apoptosis in melanoma cell lines (FIG. 5B, right side panel). FIG. 5C is a graph showing the percentage of dead cells in populations of normal melanocytes and melanoma cells treated with the proteasome inhibitors MG-132 (10 μM) and lactacystin (10 μM). MG-132 and lactacystin failed to trigger an apoptotic response in proliferating melanocytes (FIG. 5C, left side panel). In contrast, bortezomib triggered a dose-dependant increase in apoptosis in melanoma cell lines (FIG. 5C, right side panel). FIG. 5D are phase contrast microscopy images of RJ002L melanoma cells after exposure (24 hours), to the proteasome inhibitors lactacystin (1-10 μM), MG-132 (1-10 μM) and bortezomib (0.01 to 10 μM). All three proteasome inhibitors triggered a dose-dependant increase in apoptosis in melanoma cell lines.

FIG. 6 shows the effect of proteasome inhibitors on apoptosis and induction of NOXA expression in melanoma cells, myeloma cells and normal melanocytes. FIG. 6A is a Western blot showing the effect of MG-132 and lactacystin on NOXA protein levels in melanoma cells and normal melanocytes. NOXA expression is induced by both MG-132 and lactacystin in melanoma cell lines (irrespective of p53 status), but not in normal melanocytes. FIG. 6B is a graph showing the percentage of dead cells in populations of melanoma cells transfected with NOXA-specific antisense oligonucleotides (“NOXA ASO”) or control antisense oligonucleotides (“control ASO”) and treated with either the proteasome inhibitor MG-132 or lactacystin. Knock-down of NOXA with NOXA ASO, but not control ASO, significantly reduced apoptosis induced by either MG-32 or lactacystin in melanoma cells. FIG. 6C is a Western blot showing the effect of two proteasome inhibitors, bortezomib (“Velcade” and “PS-341”) and MG-132, and a dual inhibitor, z-Leu-Leu-Nle-CHO (“GSI”) on NOXA and caspase 3 expression in myeloma cell lines. Bortezomib induces NOXA protein levels in a concentration dependent fashion in the different myeloma cell lines. At a concentration of 10 μM, bortezomib induces approximately equal NOXA levels as that produced by another proteasome inhibitor, MG-132, and the dual inhibitor z-Leu-Leu-Nle-CHO (“GSI”). FIG. 6D is a graph showing the percentage of dead cells in populations of melanoma cells transfected with NOXA-specific ASO (“NOXA ASO”) or control ASO and treated with the proteasome inhibitor bortezomib. Knock-down of NOXA with NOXA ASO, but not control ASO, significantly reduced apoptosis induced by bortezomib in RJ002L melanoma cells (p<0.02).

FIG. 7 shows that the proteasome inhibitor bortezomib induces regression of C8161 cells in melanoma xenographs. FIG. 7A is a photograph showing the clinical appearance of nude mice with subcutaneous tumors treated with PBS or bortezomib. Treatment with bortezomib reduces the average subcutaneous tumor size as compared to PBS treated lesions. FIG. 7B is a graph of tumor weight of the bortezomib and PBS-treated lesions. The reduction in weight of subcutaneous tumors removed from bortezomib treated lesions versus control PBS treated lesions was significant (p<0.05). FIG. 7C is a microscopic image of H&E stained tumor slices from PBS and bortezomib treated tumors. The PBS-treated tumor contains an expansile mass of viable human C8161 melanoma cells filling the upper dermis (FIG. 7C, left panel). The bortezomib treated tumor contains scattered apoptotic cells (FIG. 7C, right panel). Arrows indicate cells undergoing apoptosis.

FIG. 8 shows the effect the proteasome inhibitor bortezomib on anti- and pro-apoptotic proteins including NOXA in melanoma cell and normal melanocytes. FIGS. 8A and 8B are Western blots showing the effect of bortezomib on the profile of BH3-only proteins (FIG. 8A) and multiple BH-related proteins. (FIG. 8A) in the melanoma and melanocytes cells. The only BH3-only protein induced by bortezomib treatment of melanoma cells was NOXA. None of the multiple-BH related family member proteins were consistently induced in all four melanoma cells by bortezomib. Normal melanocytes responded very differently to bortezomib, and they did not exhibit NOXA induction in response to bortezomib. FIG. 8C is another Western blot, this time from whole cell protein extracts generated from subcutaneous tumors produced in Nude mice and treated with PBS or bortezomib. Tumors of C8161 melanoma cells injected with PBS did not contain detectable NOXA, but bortezomib-treated tumors did induce NOXA. Actin was used as a loading control for all of the Western blots in FIG. 8. FIG. 8D is a Northern blot of NOXA mRNA using RNA extracted from C8161 melanoma cells before and after treatment with bortezomib (1 μM). The housekeeping gene GADPH was used as a loading control. Treatment of the C8161 melanoma cells with bortezomib increased NOXA transcription as early as 2 h after treatment. FIG. 8E is a Northern blot of NOXA mRNA using RNA extracted from four different melanoma cells lines before and after treatment with bortezomib (1 μM). The housekeeping gene GADPH was used as a loading control. Some of the cell lines constitutively expressed low levels of NOXA, but treatment with bortezomib caused a several-fold induction in NOXA transcription in all of these melanoma cell lines.

FIG. 9 shows the differential induction of NOXA in melanocytes, melanoma cells and osteosarcoma cells in the presence of the proteasome inhibitors MG-132, lactacystin and bortezomib. FIG. 9A is a Western blot showing the effect of MG-132 and lactacystin on p53 and NOXA levels in melanocytes. Proteasome inhibition in the melanocytes enhanced the p53 levels, but NOXA was not induced in any of the normal melanocytes in the presence of MG-132 or lactacystin. FIG. 9B is a Western blot showing the effect of MG-132 and lactacystin on p53 and NOXA levels in melanoma cells. Proteasome inhibition in the melanoma cells enhanced the p53 levels in RJ002L and C8161 cells, but no p53 was detected before or after treatment in MUM2B cells carrying homozygous p53 mutations. In all three melanoma cell lines, MG-132 and lactacystin induced high NOXA levels. FIG. 9C is a Western blot showing the effect of p53 levels on bortezomib-induced p53, MDM2, GADD45 and NOXA levels in melanoma cells. Knock-down of p53 levels using p53 siRNA not only reduced constitutive and bortezomib-induced p53 protein levels, but also significantly reduced bortezomib-induced MDM2 and GADD45 levels accompanied by only a slight reduction in bortezomib-induced NOXA levels. FIG. 9D is a Western blot showing the effect of bortezomib on NOXA levels in p53 null osteosarcoma cells. Proteasome inhibition in the osteosarcoma cells using bortezomib induced high NOXA levels, even in the absence of p53. Actin was used as a loading control for all of the Western blots in FIG. 9.

FIG. 10 shows the decrease in proteasome inhibitor-induced apoptosis in melanoma cells treated with NOXA-specific antisense oligonucleotide (“NOXA ASO”). FIGS. 10A, 10B and 10C contain a graph showing the number dead cells (left panel) and a Western blot showing NOXA levels (right panel) in RJ002L (FIG. 10A), MUM2B (FIG. 10B) and C8161 (FIG. 10C) melanoma cells pretreated with control antisense oligonucleotide (ASO) or NOXA-specific ASO NOXA ASO) before treatment with the proteasome inhibitors MG-132, lactacystin and bortezomib. The Western blots confirm that the NOXA ASO, but not the control ASO, reduced the NOXA levels in the cells. The graphs show that the NOXA ASO reduced the apoptotic response of all three proteasome inhibitors tested in all three melanoma cells lines tested. Actin was used as a loading control for all of the Western blots in FIG. 10.

FIG. 11 shows that withdrawal of serum from melanoma cells, which renders the cells quiescent, does not reduce proteosome inhibitor-induced NOXA expression or apoptotic response. FIG. 11A is a graph showing the proliferation of C8161 melanoma cells in the presence and absence of 10% fetal calf serum (“FCS”). By days 2 and 3, serum withdrawal significantly reduced the proliferation of the melanoma cells. FIG. 11B is a Western blot showing the effect of the proteosome inhibitors MG-132 and bortezomib on NOXA and p21 levels in either rapidly proliferating C8161 melanoma cells (10% FCS) or growth arrested C8161 melanoma cells (no serum). Growth arrest by serum withdrawal was confirmed by induction of p21. Both MG-132 and bortezomib caused strong induction of NOXA in either proliferating or serum-deprived melanoma cells. Actin was used as a loading control for the Western blot. FIG. 11C is a graph showing the relative apoptotic response induced in C8161 melanoma cells grown in either the presence or absence of 10% FCS and in the absence or presence of bortezomib. Minimal spontaneous apoptosis occurs even after 2 days of serum starvation, but bortezomib causes a prominent apoptotic response, irrespective of the serum status of the culture.

FIG. 12 is a graph showing induction of apoptosis in RPMI8226 myeloma cells by the proteasome inhibitor bortezomib. Doses as low as 0.01 μM bortezomib trigger significant apoptosis (p<0.05).

FIG. 13 shows the chemical structure of N-benzyloxycarbonyl-leucyl-leucyl-norleucinal (z-Leu-Leu-Nle-CHO) (FIG. 13A) and its peptidyl boronic acid derivative z-Leu-Leu-Nle-B(OH)₂ (FIG. 13B).

FIG. 14 shows potentiation of the effects of cisplatin-induced cytotoxicity and apoptosis in human cervical cancer cells by Notch-1 silencing via siRNA. FIG. 14A is a Western blot which shows that the Notch-1 siRNA is effective at silencing Notch-1 expression in the CaSki cervical cancer cells. FIG. 14B is a graph of cell growth inhibition in CaSki cervical cancer cells caused by the combination of Notch-1 or control (“scrambled”) siRNA and cisplatin. Inhibition of Notch-1 expression using the Notch-1 siRNA potentiates the cell growth inhibitory effect of cisplatin in the cervical cancer cells. FIG. 14C is a graph of the effect of Notch-1 inhibition on cisplatin-induced apoptosis. Inhibition of Notch-1 expression using Notch-1 siRNA significantly enhances cisplatin-induced apoptosis in cervical cancer cells.

FIG. 15 shows potentiation of the cytotoxic effect of cisplatin and induction of apoptosis in human cervical cancer cells by z-Leu-Leu-Nle-CHO. FIG. 15A is a Western blot of the effect of z-Leu-Leu-Nle-CHO (“GSI”) on Notch-1 expression in cervical cancer cells. The dual inhibitor z-Leu-Leu-Nle-CHO (“GSI”) caused a dose-dependent reduction in cleaved Notch-1, with a corresponding accumulation in transmembrane Notch-1. FIG. 15B is a graph showing the effects of z-Leu-Leu-Nle-CHO (“GSI”) on cell growth inhibition in cervical cancer cells. z-Leu-Leu-Nle-CHO (“GSI”) alone is cytotoxic for CaSki cells, with an IC₅₀=0.64 μM. FIG. 15C is a graph showing the effects on cell growth inhibition in cervical cancer cells of a combined treatment of z-Leu-Leu-Nle-CHO (“GSI”) and cisplatin. z-Leu-Leu-Nle-CHO (“GSI”) potentiated the effects of cisplatin on cell growth inhibition. FIG. 15D is a graph showing the synergistic effect between z-Leu-Leu-Nle-CHO (“GSI”) and cisplatin, especially at low cisplatin concentrations (1-50 μM). FIG. 15E is a graph showing that the effect of z-Leu-Leu-Nle-CHO (“GSI”) on CaSki cell survival is mediated by Notch-1. CaSki cells transfected with a constitutively active Notch-1 (ICN) significantly reversed the effects of z-Leu-Leu-Nle-CHO (“GSI”) at concentrations below IC₅₀. FIG. 15F is a graph showing the relative number of apoptotic CaSki cells after treatment with various concentrations of z-Leu-Leu-Nle-CHO (“GSI”). z-Leu-Leu-Nle-CHO (“GSI”) primarily induces cell death via apoptosis. FIG. 15G is a Western blot showing the effect of z-Leu-Leu-Nle-CHO (“GSI”) on caspase 3 protein forms in cervical cancer. Cell death induced by z-Leu-Leu-Nle-CHO (“GSI”) is accompanied by dose-dependent caspase 3 activation.

FIG. 16 shows synergistic effect of gamma secretase inhibitor LY411,575 and either AKT inhibitor I or proteasome inhibitor MG-132 on cell growth inhibition in human cervical cancer cells. FIG. 16A is a graph showing the effects of the combined treatment of the gamma-secretase inhibitor LY411,575 and AKT inhibitor I on cell growth inhibition in cervical cancer cells. AKT inhibitor I potentiates the effect of the gamma-secretase inhibitor LY411,575 in cytotoxicity assays. FIG. 16B is a graph showing the synergistic effect between LY411,575 and AKT inhibitor I. FIG. 16C is a graph showing the effects of the combined treatment of the gamma-secretase inhibitor LY411,575 and MG-132 on cell growth inhibition in cervical cancer cells. MG-132 potentiates the effect of the gamma-secretase inhibitor LY411,575 in cytotoxicity assays. FIG. 16D is a graph showing the striking synergistic effect between LY411,575 and MG-132.

FIG. 17 shows that genetic silencing of Notch-1 inhibits proliferation of normal breast cells and MDA-MB231 breast cancer cells, and has an anti-neoplastic effect on MDA-MB231 breast cancer cells. FIG. 17A is a graph showing the effect of Notch-1 and Notch-4 silencing using Notch-1 siRNA (“Notch-1i”) on proliferation of normal breast cells. Notch-1 siRNA (“Notch-1i”) inhibits proliferation of normal breast cells. FIG. 17B is a graph showing the effect of Notch-1 and Notch-4 silencing using Notch-1 siRNA (“Notch-1i”) and Notch-4 siRNA (“Notch-4i”) on proliferation of MDA-MB231 breast cancer cells. Notch-1 siRNA (“Notch-1i”) inhibits proliferation of MDA-MB231 breast cancer cells. Notch-4 siRNA (“Notch-4i”) caused an even stronger anti-proliferative effect in MDA-MB231 cells than Notch-1 silencing. FIG. 17C is a Western blot showing Notch-4, but not Notch-1, silencing by Notch-4 siRNA (“Notch-4i”) in MDA-MB231 cells. FIG. 17D is a graph showing the effect of Notch-1 and Notch-4 silencing using Notch-1 siRNA (“Notch-1i”) and Notch-4 siRNA (“Notch-4i”) on extracellular matrix invasion of MDA-MB231 breast cancer cells. Notch-1 (“Notch-1i”) and Notch-4 (“Notch-4i”) silencing significantly inhibited extracellular matrix invasion, with similar potencies.

FIG. 18 shows that pharmacological inhibition of Notch signaling inhibits proliferation and extracellular matrix invasion in breast cancer cells. FIG. 18A is a Western blot showing Notch-1 silencing (as measure by N^{TM} and N^{IC} levels) in MDA-MB231 cells by the gamma secretase inhibitor IL-X. GADPH was used as a loading control. FIG. 18B is graph showing the effect of Notch-1 silencing using various concentrations of the gamma secretase inhibitor IL-X on proliferation of MDA-MB231 breast cancer cells. Notch-1 silencing by IL-X inhibits proliferation of MDA-MB231 breast cancer cells only above 100 μM. FIG. 18C is a graph showing the effect of Notch-1 silencing using various concentrations of the gamma secretase inhibitor IL-X on extracellular matrix invasion of MDA-MB231 breast cancer cells. Notch-1 silencing by 25 μM IL-X significantly reduced in vitro matrix invasion of MDA-MB231 cells. FIG. 18D is a Western blot showing Notch-1 silencing in MDA-MB231 cells by the dual inhibitor z-Leu-Leu-Nle-CHO (“GSI”), as evidenced by a dose-dependent decrease in N^{IC} and relative accumulation of N^{TM}. GADPH was used as a loading control. FIG. 18E is graph showing the effect of Notch-1 silencing using various concentrations of the dual inhibitor z-Leu-Leu-Nle-CHO (“GSI”) on proliferation of MDA-MB231 breast cancer cells. Notch-1 silencing by z-Leu-Leu-Nle-CHO (“GSI”) inhibits proliferation of MDA-MB231 breast cancer cells only above 100 μM. FIG. 18F is a graph showing that the effect of z-Leu-Leu-Nle-CHO (“GSI”) on MDA-MB231 cell survival is mediated by Notch-1. CaSki cells transfected with a constitutively active Notch-1 (“Notch-1”) significantly reversed the effects of z-Leu-Leu-Nle-CHO (“GSI”). FIG. 18G is a graph showing the effects of z-Leu-Leu-Nle-CHO (“GSI”) on cell growth inhibition in MDA-MB231 breast cancer cells. z-Leu-Leu-Nle-CHO (“GSI”) alone is cytotoxic for MDA-MB231 cells, with an IC₅₀=1.30 μM. FIG. 18H is a graph showing the effects of z-Leu-Leu-Nle-CHO (“GSI”) on cell growth inhibition in T47D:C42 breast cancer cells. z-Leu-Leu-Nle-CHO (“GSI”) alone is cytotoxic for T47D:C42 cells, with an IC₅₀=0.38 μM. FIG. 18I is a graph showing the effects of z-Leu-Leu-Nle-CHO (“GSI”) on cell growth inhibition in T47D:A18 breast cancer cells. z-Leu-Leu-Nle-CHO (“GSI”) alone is cytotoxic for T47D:A18 cells, with an IC₅₀=0.57 μM.

FIG. 19 shows that pharmacological or genetic inhibition of Notch signaling causes growth arrest in G2/M in breast cancer cells. FIG. 19A is a graph showing the cell cycle distribution of MDA-MB231 cells treated with z-Leu-Leu-Nle-CHO (“GSI”). Pharmacological inhibition of Notch signaling using z-Leu-Leu-Nle-CHO (“GSI”) caused growth arrest in G2/M in the breast cancer cells and a corresponding decrease in the number of cells in G1 and S. FIG. 19B is a graph showing the cell cycle distribution of MDA-MB231 cells treated with Notch-1 siRNA (“Notch-1i”) or control siRNA (“control”). Genetic inhibition of Notch signaling using Notch-1 siRNA (“Notch-1i”), but not control siRNA (“control”), caused growth arrest in G2/M in the breast cancer cells and a corresponding decrease in the number of cells in G1 and S. FIG. 19C is a graph showing the cell cycle distribution of MDA-MB231 cells treated with Notch-4 siRNA (“Notch-4i”) or control siRNA (“control”). Genetic inhibition of Notch signaling using Notch-4 siRNA (“Notch-4i”), but not control siRNA (“control”), caused growth arrest in G2/M in the breast cancer cells and a corresponding decrease in the number of cells in G1 and S.

FIG. 19D shows representative raw data from flow cytometry experiments with the Notch-1 siRNA (“Notch-1i”)- and Notch-4 siRNA (“Notch-4i”)-treated cells. Numbers above the graphs indicate percentages of cells in subG1, G1, S and G2/M, respectively. FIG. 19E shows that results of a Northern blot analysis to determine the effects of Notch-1 inhibition by Notch-1 siRNA (“Notch-1i”) on the expression of different cyclins. MDA-MB231 cells treated with Notch-1 siRNA (“Notch-1i”) express reduced levels of cyclins A and B1. L32 (rRNA) and GAPDH were internal controls. Additional controls were untreated cells and tRNA (non-specific protection control). FIG. 19F is a Western blot showing the effect of Notch-1 inhibition by Notch-1 siRNA (“Notch-1i”) on cyclin B, Cdkl and cyclin A expression in MDA-MB231 cells. Notch-1 silencing by Notch-1 siRNA (“Notch-1i”) dramatically reduced the levels of cyclin B1 and cyclin A proteins. FIG. 19G is a Western blot showing the effect of Notch-1 inhibition by Notch-1 siRNA (“Notch-1i”) on E2F-1 and p21 expression in MDA-MB231 cells. Notch-1 silencing by Notch-1 siRNA (“Notch-1i”) causes accumulation of E2F-1 at 48 h (“Day 2”) and down regulation of p21 at 24 h (“Day 1”). FIG. 19H is a Western blot showing the effect of z-Leu-Leu-Nle-CHO (“GSI”) (0.5 or 1 μM, 24 h) treatment on cyclin B and cyclin A expression in MDA-MB231 cells. z-Leu-Leu-Nle-CHO (“GSI”) dramatically reduced the levels of cyclin A protein at 0.5 μM, whereas cyclin B was decreased at 1 μM.

FIG. 20 shows that pharmacological inhibition of Notch-1 potentiates Paclitaxel-induced inhibition breast cancer cell growth. FIG. 20A is a graph showing the effects on cell growth inhibition in MDA-MB-231 breast cancer cells of a combined treatment of z-Leu-Leu-Nle-CHO (“GSI”) and Paclitaxel. z-Leu-Leu-Nle-CHO (“GSI”) moderately potentiated the effects of Paclitaxel on cell growth inhibition. FIG. 20B is a graph showing the effects on cell growth inhibition in T47D:C42 breast cancer cells of a combined treatment of z-Leu-Leu-Nle-CHO (“GSI”) and Paclitaxel. z-Leu-Leu-Nle-CHO (“GSI”) potentiated the effects of Paclitaxel on cell growth inhibition. FIG. 20C is a graph showing the effects on cell growth inhibition in T47D:A18 breast cancer cells of a combined treatment of z-Leu-Leu-Nle-CHO (“GSI”) and Paclitaxel. z-Leu-Leu-Nle-CHO (“GSI”) potentiated the effects of Paclitaxel on cell growth inhibition.

### DETAILED DESCRIPTION OF PREFERRED EMBODIMENTS

The invention provides methods and compositions, particularly pharmaceutical compositions, for treating cancer. In particular embodiments, the methods and compositions, particularly pharmaceutical compositions, of the invention induce apoptosis preferentially in cancer cells and not normal (i.e., non-cancer) cells. In alternative embodiments, the methods and compositions, particularly pharmaceutical compositions, of the invention inhibit growth of cancer cells, most particularly and preferentially without inhibiting growth of normal (i.e., non-cancer) cells.

In one aspect, the invention provides methods for inducing apoptosis in cancer cells comprising the step of contacting the cancer cells with a gamma secretase inhibitor and a proteasome inhibitor in combination and in amounts and for a period of time sufficient to induce apoptosis.

As used herein, the term “inducing apoptosis in cancer cells” refers to any increase in the percentage of cancer cells killed within a population of cancer cells. As one of skill in the art will appreciate, benefit may be conferred without achieving 100% killing.

As used herein, the term “gamma secretase inhibitor” is any compound with the ability to inhibit the activity of gamma-secretase, a membrane-bound protease that cleaves the transmembrane region of amyloid precursor protein (APP).

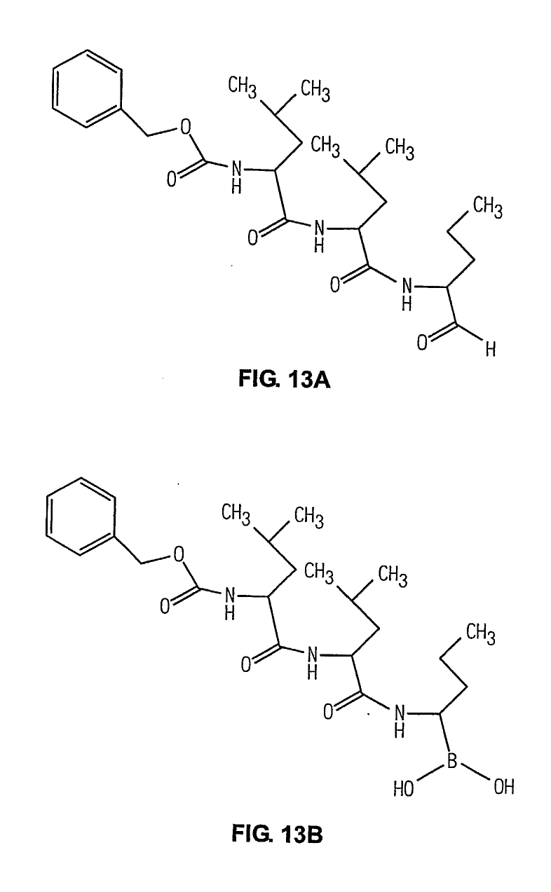

A preferred gamma secretase inhibitor for use in the present invention is the tripeptide aldehyde, N-benzyloxycarbonyl-leucyl-leucyl-norleucinal, referred to herein as z-Leu-Leu-Nle-CHO (FIG. 13A).

Non-limiting examples of additional gamma secretase inhibitors for use in the present invention include gamma secretase inhibitors known in the art, as well as structurally related molecules or molecules derived therefrom (for example using, inter alia, the methods and techniques described in Seiffert et al., 2000, J. Biol. Chem. 275:34086-34091; Tian et al., 2002, J. Biol. Chem. 277:31499-31505; Tian et al., 2003, J. Biol. Chem. 278:28968-28975). In general, gamma secretase inhibitors are short peptides (e.g., about two to about five amino acid residues) comprised primarily of hydrophobic amino acids or peptidomimetic agents that structurally resemble such peptides.

In addition, derivatives of the gamma secretase inhibitor z-Leu-Leu-Nle-CHO (FIG. 13A) can be used in the invention. For examples, z-Leu-Leu-Nle-CHO derivatives for use in the invention can include molecules similar to z-Leu-Leu-Nle-CHO in which the aldehyde group of z-Leu-Leu-Nle-CHO is replaced with boronate to create a peptidyl boronic acid or z-Leu-Leu-Nle-B(OH)₂ (FIG. 13B). It is well within the ability of one of ordinary skill in the art to obtain z-Leu-Leu-Nle-CHO or other gamma secretase inhibitor derivatives using any suitable synthesis. For example, one could obtain a z-Leu-Leu-Nle-CHO boronate derivative by according to methods described in described by Adams et al., 1998, Bioorganic and Medicinal Chemistry Lett. 8:333-338.

In addition to peptides, other molecules that can be used as chemical scaffolds to synthesize molecules that have the ability to inhibit gamma secretase include, without limitation, sulfonamides and benzodiazepines. One non-limiting example of a non-peptide, benzodiazepine gamma-secretase inhibitor is LY411,575 (Wong et al., 2004, J Biol. Chem. 279:12876-82.)

Other suitable z-Leu-Leu-Nle-CHO, derivatives include those having a substituent at the N-terminus other than “z”. For example, it is specifically envisioned that the N-terminus can be modified to include any acyl group. In addition, one or more of the hydrophobic amino acids of z-Leu-Leu-Nle-CHO or other peptide gamma secretase inhibitors can be replaced with another other hydrophobic amino acid (e.g., leucine, isoleucine, valine, phenylalanine, tyrosine, alanine, methoinine, threonine, beta alanine) in any combination. Further, it is envisioned that one or more of L-amino acids could be replaced with the corresponding D-amino acid or a non-naturally-occurring amino acid (e.g., phenylalanine in which the benzene ring of the side chain is substituted by one or more fluorine atoms). In alternative embodiments, the amino acids are linked by non-peptide bonds. Disulfide bonds may be added to stabilize longer peptides.

The invention also provides methods and compositions, particularly pharmaceutical compositions using a proteasome inhibitor. As used herein, the term “proteasome inhibitor” is any substance which directly or indirectly inhibits the 20S or 26S proteasome or the activity thereof.

Non-limiting examples of proteasome inhibitors for use in the present invention include peptide aldehydes (see, e.g., Stein et al., PCT Publication No. WO 95/24914 published Sep. 21, 1995; Siman et al., PCT Publication No. WO 91/13904 published Sep. 19, 1991; Iqbal et al., 1995, J. Med. Chem. 38:2276-2277), vinyl sulfones (see, e.g., Bogyo et al., 1997, Proc. Natl. Acad. Sci. USA 94:6629), .alpha.′.beta.′-epoxyketones (see, e.g., Spaltenstein et al., 1996, Tetrahedron Lett. 37:1343); peptide boronic acids (see, e.g., Adams et al., PCT Publication No. WO 96/13266 published May 9, 1996; Siman et al., PCT Publication No. WO 91/13904 published Sep. 19, 1991), and lactacystin and lactacystin analogs (see, e.g., Fenteany et al., 1994, Proc. Natl. Acad. Sci. USA 94:3358; Fenteany et al., PCT Publication No WO 96/32105 published Oct. 19, 1996), each of which is hereby incorporated by reference in its entirety. A preferred proteasome inhibitor is benzoxycarbonyl-Leucyl-Leucyl-Leucyl-aldehyde (otherwise known as MG-132). Another preferred proteasome inhibitor is bortezomib (N-(2-pyrazine)carbonyl-L-phenylalanine-L-leucine boronic acid).

As used herein, the phrase “in combination” means providing one compound in conjunction with another compound, wherein the compounds may be provided simultaneously or concurrently, sequentially or in any order. When provided simultaneously or concurrently, the compounds can be administered as a single treatment, or the compounds can be provided as separate treatments that are administered concurrently. When the compounds are administered simultaneously or concurrently, sequentially or in any order, the administration of each can be by the same method or by different methods. Any combination of a gamma secretase inhibitor, a proteasome inhibitor, a tumoricidal agent or a compound that inhibits Notch-1 gene expression or protein activity are used methods and compositions, particularly pharmaceutical compositions of the invention. Preferred embodiments utilize combinations of a gamma secretase inhibitor and a proteasome inhibitor, a gamma secretase inhibitor and a tumoricidal agent, a Notch-1 siRNA and a tumoricidal agent or a Notch-1 siRNA and a proteasome inhibitor. Particularly preferred embodiments utilize combinations of a gamma secretase inhibitor and a proteasome inhibitor, a gamma secretase inhibitor and a tumoricidal agent, a Notch-1 siRNA having the sequence identified as SEQ ID NO. 1 and a tumoricidal agent or a Notch-1 siRNA having the sequence identified as SEQ ID NO. 1 and a proteasome inhibitor.

The term “cancer” refers to unregulated or uncontrolled growth of cells resulting in a spectrum of pathological symptoms associated with the initiation or progression of tumor growth, as well as metastasis of malignant tumors. As used herein, the term “tumor” is used to refer to a new growth of tissue in which the multiplication of cells is uncontrolled and progressive. The tumor that is relevant to the invention can be benign, i.e. one that does not form metastases and does not invade and destroy adjacent normal tissue, or malignant, i.e. one that invades surrounding tissues, is capable of producing metastases, may recur after attempted removal, and is likely to cause death of the host.

Cancers that are treatable by the present invention and cancer cells suitable for use with the present invention include, but are not limited to, melanomas, multiple myelomas, prostate cancer, osteosarcomas, breast cancer, ovarian cancer, pancreatic cancer, colon cancer, acute lymphoblastic T- or B-cell leukemia, cervical cancer, endometrial cancer, malignant mesothelioma, carcinomas of the head and neck and thyroid, renal cancer, lung cancer, anaplastic non-Hodgkin lymphoma, Hodgkin lymphoma, acute myeloblastic leukemia, chronic lymphoid leukemia, gliomas, medulloblastomas, and neuroblastomas.

Cancer cells useful in the inventive methods for inducing apoptosis in cancer cells as disclosed and claimed herein are preferably melanoma, multiple myeloma, prostate cancer, osteosarcoma, breast cancer, or cervical cancer cells.

In one embodiment, a single dual inhibitor compound is used in the disclosed methods for inducing apoptosis in cancer cells. The terms “dual inhibitor compound” and “dual inhibitor” as used herein are compounds that possess both gamma secretase inhibitor and proteasome inhibitor activities. Non-limiting examples of dual inhibitor compounds for use in the invention are leucyl-leucyl-norleucinal, N-benzyloxycarbonyl-leucyl-leucyl-norleucinal and N-benzyloxycarbonyl-leucyl-leucyl-leucinal (also known as MG-132).

In certain other embodiments, a gamma secretase inhibitor and a proteasome inhibitor useful in the methods of inducing apoptosis in cancer cells of the invention are separate and distinct compounds.

In the Examples below, apoptosis was evaluated using a commercially available kit and flow cytometry, or cell DNA content and flow cytometry. As one of skill in the art will appreciate, any suitable means of detecting apoptosis may be used in the method of the invention.

In other aspects, the invention provides methods for inhibiting cell growth in cancer cells comprising the step of contacting the cancer cells with a gamma secretase inhibitor and a proteasome inhibitor in combination and in amounts and for a period of time sufficient to inhibit cell growth.

As used herein, the term “cell growth in cancer cells” is used to refer to the growth of a tumor cell. Assaying for cell growth inhibition in cancer cells can be accomplished by any method known the art, including but not limited to monitoring protein content in a cell culture using the crystal violet staining method as described by Skehan et al., 1990, J. Natl. Cancer Inst. 82:1107-1112 and Prochaska et al., 1988, Anal. Biochem. 169:328-336.

Cancer cells useful in the methods for inhibiting cell growth in cancer cells disclosed herein are preferably melanoma, multiple myeloma, prostate cancer, osteosarcoma, breast cancer, or cervical cancer cells.

In certain embodiments, a single dual inhibitor compound is used in the inventive methods for inhibiting cell growth in cancer cells as disclosed herein. Non-limiting examples of dual inhibitor compounds for use in these methods of the invention are leucyl-leucyl-norleucinal, N-benzyloxycarbonyl-leucyl-leucyl-norleucinal and N-benzyloxycarbonyl-leucyl-leucyl-leucinal (also known as MG-132).

In certain other embodiments, the gamma secretase inhibitor and the proteasome inhibitor used in the methods of inhibiting cell growth in cancer cells disclosed herein are separate and distinct compounds.

In yet other aspects, the present invention provides methods for inducing apoptosis in cancer cells comprising the step of contacting the cancer cells with a gamma secretase inhibitor and a tumoricidal agent in combination and in amounts and for a period of time sufficient to induce apoptosis, wherein the tumoricidal agent is a cytotoxic drug or radiation.

Examples of cytotoxic drugs include, in general and without limitation, microtubule-stabilizing agents such as paclitaxel (TAXOL®), docetaxel (TAXOTERE®), epothilone A, epothilone B, desoxyepothilone A, desoxyepothilone B or their derivatives); microtubule-disruptor agents; alkylating agents, for example, nitrogen mustards, ethyleneimine compounds, alkyl sulfonates and other compounds with an alkylating action such as nitrosoureas, cisplatin, and dacarbazine; anti-metabolites, for example, folic acid, purine or pyrimidine antagonists; epidophyllotoxin; an antineoplastic enzyme; a topoisomerase inhibitor; procarbazine; mitoxantrone; platinum coordination complexes, including but not limited to cisplatin and carboplatin; biological response modifiers and growth inhibitors; mitotic inhibitors, for example, vinca alkaloids and derivatives of podophyllotoxin; cytotoxic antibiotics; hormonal/anti-hormonal therapeutic agents, haematopoietic growth factors and antibodies (such as trastuzumab, also known as HERCEPTIN), proteasome inhibitors, AKT inhibitors, mitotic poisons, DNA damaging agents, growth factor receptor antagonists, or STAT3 inhibitors.

Exemplary classes of cytotoxic drugs include, for example, the anthracycline family of drugs, the vinca drugs, the mitomycins, the bleomycins, the cytotoxic nucleosides, the taxanes, the epothilones, discodermolide, the pteridine family of drugs, diynenes and the podophyllotoxins. Particularly useful members of those classes include, for example, doxorubicin, caminomycin, daunorubicin, aminopterin, methotrexate, methopterin, dichloro-methotrexate, mitomycin C, porfiromycin, 5-fluorouracil, 6-mercaptopurine, gemcitabine, cytosine arabinoside, podophyllotoxin or podo-phyllotoxin derivatives such as etoposide, etoposide phosphate or teniposide, melphalan, vinblastine, vincristine, leurosidine, vindesine, leurosine, paclitaxel and the like. Other useful antineoplastic agents include estramustine, cisplatin, carboplatin, cyclophosphamide, bleomycin, tamoxifen, ifosamide, melphalan, hexamethyl melamine, thiotepa, cytarabin, idatrexate, trimetrexate, dacarbazine, L-asparaginase, dactinomycin, mechlorethamine (nitrogen mustard), streptozocin, cyclophosphamide, carnustine (BCNU), lomustine (CCNU), procarbazine, mitomycin, cytarabine, etoposide, methotrexate, bleomycin, chlorambucil, camptothecin, CPT-11, topotecan, ara-C, bicalutamide, flutamide, leuprolide, pyridobenzoindole derivatives, interferons and interleukins. Particular examples of cytotoxic drugs, or chemotherapeutic agents, are described, for example, by Stewart in “Nausea and Vomiting: Recent Research and Clinical Advances”, (Kucharczyk et al., eds.), CRC Press Inc.: Boca Raton, Fla., USA (1991), pp. 177-203, especially page 188. See also, Gralla et al., 1984, Cancer Treatme t Reports 68: 163-172.

In preferred embodiments, the gamma secretase inhibitor used in combination with a tumoricidal agent is N-benzyloxycarbonyl-leucyl-leucyl-norleucinal. In other preferred embodiments, the tumoricidal agent is a proteasome inhibitor, an AKT inhibitor, a mitotic poison, a DNA damaging agent, a growth factor receptor antagonist, or a STAT3 inhibitor, and is used in combination with N-benzyloxycarbonyl-leucyl-leucyl-norleucinal to induce apoptosis in cancer cells. In particularly preferred embodiments, the tumoricidal agent is a platinum coordination complex, AKT inhibitor I, MG-132 or Paclitaxel, and is used in combination with N-benzyloxycarbonyl-leucyl-leucyl-norleucinal to induce apoptosis in cancer cells.

In certain embodiments, cancer cells useful in the methods of inducing apoptosis in cancer cells disclosed herein are melanoma, multiple myeloma, prostate cancer, osteosarcoma, breast cancer, or cervical cancer cells.

In additional aspects, the invention provides methods for inhibiting cell growth in cancer cells comprising the step of contacting the cancer cells with a gamma secretase inhibitor and a tumoricidal agent in combination and in amounts and for a period of time sufficient to inhibit cell growth, wherein the tumoricidal agent is a cytotoxic drug or radiation.

In preferred embodiments, the gamma secretase inhibitor used in said methods is N-benzyloxycarbonyl-leucyl-leucyl-norleucinal. In preferred embodiments, the tumoricidal agent is a proteasome inhibitor, an AKT inhibitor, a mitotic poison, a DNA damaging agent, a growth factor receptor antagonist, or a STAT3 inhibitor. Preferably one or a plurality of said tumoricidal agents is used in combination with N-benzyloxycarbonyl-leucyl-leucyl-norleucinal to inhibiting cell growth in cancer cells. In particularly preferred embodiments, the tumoricidal agent is a platinum coordination complex, AKT inhibitor I, MG-132 or Paclitaxel, and one or a plurality of said tumoricidal agents is used in combination with N-benzyloxycarbonyl-leucyl-leucyl-norleucinal to inhibiting cell growth in cancer cells.

Cancer cells useful in the disclosed methods for inhibiting cell growth in cancer cells are preferably melanoma, multiple myeloma, prostate cancer, osteosarcoma, breast cancer, or cervical cancer cells.

In yet other aspects, the invention provides methods for inducing apoptosis in cancer cells comprising the step of contacting the cancer cells with a compound that inhibits Notch-1 gene expression or protein activity and a tumoricidal agent in combination, wherein the tumoricidal agent is a cytotoxic drug or radiation in amounts and for a period of time sufficient to induce apoptosis. In preferred embodiments, the compound that inhibits Notch-1 gene expression or protein activity is a Notch-1 siRNA, more particularly SEQ ID NO. 1.

The Notch signaling network plays an important role in cell fate determination. Notch receptors regulate cell differentiation, proliferation and apoptosis during intercellular contact, as a consequence of activation of the Delta and Jagged/Serrate families by transmembrane ligands. Notch precursor proteins are cleaved by a furin-like protease to generate mature heterodimers comprised of a transmembrane subunit (N^{TM}) non-covalently associated with an extracellular subunit (N^{EC}) that contains EGF-like repeats. Upon ligand binding, the transmembrane subunit is cleaved by an extracellular disintegrin metalloprotease and a presenilin-1 (PS-1)-dependent gamma-secretase. Cleavage of the transmembrane subunit releases a cytoplasmic subunit (N^{IC}), which migrates to the nucleus and regulates the function of ubiquitous transcription factor CBF-1, which in turn modulates the expression of multiple targets including several transcription factors, thereby modulating cell fate decisions.

As described in the Examples below, Notch inhibitors, including Notch siRNA, small peptide gamma secretase inhibitors, and non-peptide gamma secretase inhibitors, work synergistically with DNA damaging agents such as platinum coordination complexes, AKT inhibitors, the proteasome inhibitor MG-132, or the mitotic poison Paclitaxel to inhibit the growth of or promote the death of cancer cells. These results are inconsistent with expectations reasonably held by one of ordinary skill in the art in a number of respects. First, proteasome inhibitors block protein degradation, including degradation of Notch. Therefore, the skilled worker would expect that proteasome inhibitors to prevent the degradation of N^{IC}, thereby antagonizing Notch inhibitors. Furthermore, Notch activation resulted in an increase in cyclin A and B. Because proteasome inhibitors block degradation of cyclin B, one would expect that the proteasome inhibitors would counter the reduction of cyclin A and B caused by Notch inhibitors. Reduced levels of cyclin A and B would delay entry of the cell into mitosis, thereby causing the cell to be retained in the S-phase or G2 phase, thus antagonizing the effects of mitotic poisons, which work on cells in mitosis.

In this aspect of the invention, Notch-1 expression is inhibited by a short interfering RNA (siRNA) through RNA interference (RNAi). RNA interference (RNAi) is a mechanism of post-transcriptional gene silencing in which double-stranded RNA (dsRNA) corresponding to a gene (or coding region) of interest is introduced into a cell or an organism, resulting in degradation of the corresponding mRNA. The RNAi effect persists for multiple cell divisions before gene expression is regained. RNAi is therefore an extremely powerful method for making targeted knockouts or “knockdowns” at the RNA level. RNAi has proven successful in human cells, including human embryonic kidney and HeLa cells (see, e.g., Elbashir et al., 2001, Nature 411:494-8).

Functional siRNAs are 21-23 nucleotides in length and are produced by a cell in response to the introduction of dsRNA. The siRNA duplexes bind to a nuclease complex to form what is known as the RNA-induced silencing complex, or RISC. The RISC targets the homologous transcript by base pairing interactions between one of the siRNA strands and the endogenous mRNA. It then cleaves the mRNA.about.12 nucleotides from the 3′ terminus thereof (reviewed in Sharp et al., 2001, Genes Dev 15: 485-490; and Hammond et al., 2001, Nature Rev Gen 2: 110-119).

RNAi technology in gene silencing utilizes conventional molecular biology methods. dsRNA corresponding to the sequence from a target gene to be inactivated can be produced by standard methods, e.g., by simultaneous transcription of both strands of a template DNA (corresponding to the target sequence) with T7 RNA polymerase. Kits for production of dsRNA for use in RNAi are available commercially, e.g., from New England Biolabs, Inc. (Beverley, Mass.) Methods of transfecting dsRNA or plasmids engineered to make dsRNA are routine in the art. In this invention, any siRNA targeting the human Notch-1 gene can be used. In a preferred embodiment, the siRNA has a sequence identified as 5′-AAG TGT CTG AGG CCA GCA AGA-3′ (SEQ ID NO. 1).

The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52: 456; Sambrook et al., 2001, MOLECULAR CLONING: A LABORATORY MANUAL, 3d ed., Cold Spring Harbor Laboratory Press, Cold Spring Harbor, N.Y.; Davis et al., 1986, BASIC METHODS IN MOLECULAR BIOLOGY (Elsevier); and Chu et al., 1981, Gene 13: 197. Such techniques can be used to introduce the Notch-1 siRNA of the invention into suitable host cells.

In a preferred embodiment, tumoricidal agents useful in the methods of the invention in combination with a compound that inhibits Notch-1 gene expression or protein activity, particularly Notch-1 siRNA, more particularly SEQ ID NO. 1, include cytotoxic drugs that can be a proteasome inhibitor, an AKT inhibitor, a mitotic poison, a DNA damaging agent, a growth factor receptor antagonist, or a STAT3 inhibitor. In a particularly preferred embodiment, the tumoricidal agent is a platinum coordination complex, AKT inhibitor I, MG-132 or Paclitaxel.

Cancer cells useful in the methods of the invention for inducing apoptosis in cancer cells by contacting the cancer cells with a compound that inhibits Notch-1 gene expression or protein activity, particularly Notch-1 siRNA, more particularly SEQ ID NO. 1 and a tumoricidal agent in combination are preferably melanoma, multiple myeloma, prostate cancer, osteosarcoma, breast cancer, or cervical cancer cells.

In other aspects, the invention provides methods for inhibiting cell growth in cancer cells comprising the step of contacting the cancer cells with a compound that inhibits Notch-1 gene expression or protein activity and a tumoricidal agent in combination, wherein the tumoricidal agent a cytotoxic drug or radiation in amounts and for a period of time sufficient to inhibiting cell growth.

In preferred embodiments, the tumoricidal agent is a proteasome inhibitor, an AKT inhibitor, a mitotic poison, a DNA damaging agent, a growth factor receptor antagonist, or a STAT3 inhibitor. In a particularly preferred embodiment, the tumoricidal agent is a platinum coordination complex, AKT inhibitor I, MG-132 or Paclitaxel. In preferred embodiments, the compound that inhibits Notch-1 gene expression or protein activity is a Notch-1 siRNA, more particularly 5′-AAG TGT CTG AGG CCA GCA AGA-3′ (SEQ ID NO. 1).

Cancer cells useful in the inventive methods for inhibiting cell growth in cancer cells by contacting said cancer cells with a compound that inhibits Notch-1 gene expression or protein activity, particularly Notch-1 siRNA, more particularly SEQ ID NO. 1 and a tumoricidal agent in combination are preferably melanoma, multiple myeloma, prostate cancer, osteosarcoma, breast cancer, or cervical cancer cells.

As described in detail below, certain gamma secretase inhibitors, preferably tripeptide aldehydes having the structure N-benzyloxycarbonyl-leucyl-leucyl-norleucinal, or z-Leu-Leu-Nle-CHO, induce NOXA gene expression in cancer cells. NOXA is a pro-apoptotic protein that is a member of the Bcl-2 family, and is further characterized as a BH3-only protein as it contains only the third of four Bcl-2 Homology (BH) domains. Induction of NOXA, a proapoptotic molecule, by z-Leu-Leu-Nle-CHO is correlated with an increase in apoptosis, even in cells that are generally regarded as being apoptosis-resistant.

In addition to the dual inhibitor N-benzyloxycarbonyl-leucyl-leucyl-norleucinal (z-Leu-Leu-Nle-CHO), proteasome inhibitors such as lactacystin, MG-132 and bortezomib also have the ability to induce NOXA in cancer cells, and NOXA induction by proteasome inhibitors was correlated with an increase in apoptosis.

The induction of apoptosis in z-Leu-Leu-Nle-CHO- or proteasome inhibitor-treated cancer cells was specific for NOXA induction, because melanoma cells transformed with an antisense highly specific for NOXA showed significantly-reduced (p≦0.02) apoptosis induction were significantly reduced but were not reduced in similarly treated cells containing a nonspecific control oligonucleotide.

NOXA induction by these compounds induced apoptosis even in cells known to be p53-deficient. These results indicate that compounds capable of inducing NOXA gene expression or activity are useful in inducing apoptosis in cancer cells, particularly p53-deficient cancer cells that are typically resistant to the apoptosis-promoting effects of conventional chemotherapeutic drugs. Identification of NOXA induction as a reliable marker for apoptosis induction also provides methods for screening and identifying novel and useful chemotherapeutic agents. Accordingly, in certain aspects, the invention provides methods for evaluating the ability of a test molecule to induce NOXA in a cancer cell comprising contacting the cell with the test molecule and determining whether the cell exhibits an increase in NOXA, relative to a control cell (i.e., a like cell not contacted with the test molecule). The test molecules may be evaluated individually or in combination with one or more other molecules or treatments. Conveniently, an array of test molecules may be evaluated simultaneously as, for example, in a high throughput assay.

In yet other aspects, the present invention provides methods for screening compounds for NOXA gene expression-inducing ability in cancer cells comprising:
(a) contacting cancer cells with a culture media in the presence and absence of a test compound;
(b) assaying the cells of step (a) for NOXA gene expression;
(c) comparing NOXA gene expression assayed in step (b) from cells contacted with culture media in the presence of the test compound with NOXA gene expression from cells contacted with culture media in the absence of the test compound; and
(d) identifying a compound that induces NOXA gene expression when NOXA gene expression is higher in cells contacted in the presence of the test compound than in cells contacted in the absence of the test compound.

These methods can employ a single type of cancer cell, or a panel comprising a plurality of different types of cancer cells. The Examples below describe the use of numerous cell lines in evaluating compounds for the ability to induce NOXA, some of which are commercially available from, for example, the American Type Culture Collection, and others that are early-passage cell lines derived from tumor that have spent relatively short times in culture, which are thus both useful in the disclosed screening methods for identifying compounds that inhibit gene expression or activity. These methods can be performed in any standard cell culture conditions, as known by one of skill in the art. For example, a RJ002L melanoma cell lines can be maintained in RPMI supplemented with 10% FBS. Suitable cell culture conditions for other cancer cell lines and cancer cell types are well known in the art.

Although the invention provides methods that are particularly useful for screening compounds effective in inducing NOXA in apoptosis-resistant cells, the methods may employ any cancer cell type, without regard to the existence of other treatments or methods for inducing apoptosis in the cancer cell. Cancer cells useful in the methods of screening compounds for NOXA gene expression-inducing ability in cancer cells are melanoma cells, myeloma cells, prostate cancer cells, osteosarcoma cells, cervical cancer cells or breast cancer cells. Cancer cells particularly useful in the screening methods of the invention are cancer cells that are deficient in p53 tumor suppressor protein.

In the Examples below, induction of NOXA expression was detected by electrophoretically separating proteins in cell lysates and subjecting the proteins to Western blot analysis. However, as one of ordinary skill in the art will appreciate, an increase in NOXA gene expression may be detected by any suitable means. It is specifically envisioned that induction of NOXA expression could be detected with or without first fractionating cellular proteins. For example, the methods described in the Examples may be adapted for use with cells grown and treated in a well (e.g., a well in a multi-well plate), followed by in-well lysis and detection of NOXA protein in a quantitative or semi-quantitative screening assay without prior purification of the NOXA protein. Optionally, NOXA may be detected following partial purification from the cell lysate by binding to immobilized anti-NOXA antibody.

It is also envisioned within the scope of these methods of the invention that induction of NOXA expression can be detected by hybridizing a detectably-labeled oligonucleotide probe specific for a portion of NOXA mRNA to cellular nucleic acids under high stringency conditions, detecting hybridization of the labeled probe, and correlating hybridization with the level of NOXA mRNA. Alternatively, NOXA mRNA may be determined using quantitative RT-PCR. It is envisioned that NOXA mRNA quantitation using any appropriate method may be conducted in a high-throughput system.

As one skilled in the art will appreciate, the methods of the invention can be used to screen any molecule or group of molecules for the ability to induce NOXA expression, including but not limited to libraries of currently available molecules (i.e., gamma secretase inhibitor libraries or proteasome inhibitor libraries), libraries of modified proteasome inhibitors, and libraries of modified gamma secretase inhibitors. Furthermore, N-benzyloxycarbonyl-leucyl-leucyl-norleucinal derivatives (z-Leu-Leu-Nle-CHO) can be tested for the ability to affect NOXA induction in cancer cells. As used herein N-benzyloxycarbonyl-leucyl-leucyl-norleucinal derivatives includes related molecules derived from or synthesized to have a structure similar to N-benzyloxycarbonyl-leucyl-leucyl-norleucinal (z-Leu-Leu-Nle-CHO) (FIG. 13A) that can be tested for its ability to affect NOXA induction. For example, suitable z-Leu-Leu-Nle-CHO derivatives would include molecules structurally similar to N-benzyloxycarbonyl-leucyl-leucyl-norleucinal in which the aldehyde group of z-Leu-Leu-Nle-CHO is replaced with boronate to create a peptidyl boronic acid or z-Leu-Leu-Nle-B(OH)₂ (FIG. 13B). It is well within the ability of one of ordinary skill in the art to obtain N-benzyloxycarbonyl-leucyl-leucyl-norleucinal or other gamma secretase inhibitor derivatives using any suitable synthesis. For example, one could obtain a N-benzyloxycarbonyl-leucyl-leucyl-norleucinal boronate derivative by according to methods described in described by Adams et al., 1998, Bioorganic and Medicinal Chemistry Lett. 8:333-338.

Other suitable N-benzyloxycarbonyl-leucyl-leucyl-norleucinal derivatives include those having a substituent at the N-terminus other than “z”. For example, it is specifically envisioned that the N-terminus could be modified to include any acyl group. In addition, it one or more of the hydrophobic amino acids of N-benzyloxycarbonyl-leucyl-leucyl-norleucinal or other peptide gamma secretase inhibitors may be replaced with another hydrophobic amino acid (e.g., leucine, isoleucine, valine, phenylalanine, tyrosine, alanine, methoinine, threonine, beta alanine) in any combination provided that each amino acid is different from at least one other amino acid comprising the molecule. Further, it is envisioned that one or more of the L-amino acids could be replaced with the corresponding D-amino acid or non-naturally-occurring amino acids (e.g., phenylalanine in which the aromatic ring is substituted with one or more fluorine atoms). In additional embodiments, the amino acids could be linked by one or more non-peptide bonds. Disulfide bonds may be added to stabilize longer peptides.

Preferably, NOXA induction stimulated by the compounds disclosed herein, or identified using the screening methods discloses herein, is accompanied by increased apoptosis in NOXA-induced cells relative to apoptosis observed in untreated or control cells. In yet other preferred embodiments, NOXA induction stimulated by the compounds disclosed herein, or identified using the screening methods discloses herein, is accompanied by the ability to inhibit cell growth in cancer cells

In another aspect, the present invention provides methods for screening a test compound for Notch-1 gene expression or activity inhibiting ability in cancer cells, the method comprising the steps of:
(a) contacting cancer cells with a culture media in the presence and absence of a test compound;
(b) assaying the cells of step (a) for Notch-1 gene expression or activity;
(c) comparing Notch-1 gene expression or activity assayed in step (b) from cells contacted with culture media in the presence of the test compound with Notch-1 gene expression or activity from cells contacted with culture media in the absence of the test compound; and
(d) identifying a compound that inhibits Notch-1 gene expression or activity when Notch-1 gene expression or activity is lower in cells contacted in the presence of the test compound than in cells contacted in the absence of the test compound.
   These inventive methods can employ a single type of cancer cell, or a panel comprising a plurality of different types of cancer cells. The Examples below describe the use of numerous cell lines in evaluating compounds for the ability to inhibit Notch-1 gene expression or activity, some of which are commercially available from, for example, the American Type Culture Collection, and others that are early-passage cell lines derived from tumor that have spent relatively short times in culture, which are thus both useful in the disclosed screening methods for identifying compounds that inhibit gene expression or activity. These methods can be performed in any standard cell culture conditions, as known by one of skill in the art. For example, a RJ002L melanoma cell lines can be maintained in RPMI supplemented with 10% FBS. Suitable cell culture conditions for other cancer cell lines and cancer cell types are well known in the art.

Cancer cells useful in the screening methods for identifying compounds that inhibit Notch-1 gene expression or activity are melanoma cells, myeloma cells, prostate cancer cells, osteosarcoma cells, cervical cancer cells or breast cancer cells. Cancer cells particularly useful in the screening methods of the invention are cancer cells that are deficient in p53 tumor suppressor protein.

In the Examples below, inhibition of Notch-1 gene expression was detected by electrophoretically separating proteins in cell lysates and subjecting the proteins to Western blot analysis. However, as one of ordinary skill in the art will appreciate, a decrease in Notch-1 gene expression may be detected by any suitable means. It is specifically envisioned that inhibition of Notch-1 gene expression could be detected with or without first fractionating cellular proteins. For example, the methods described in the Examples may be adapted for use with cells grown and treated in a well (e.g., a well in a multi-well plate), followed by in-well lysis and detection of Notch-1 protein in a quantitative or semi-quantitative screening assay without prior purification of the Notch-1 protein. Optionally, Notch-1 may be detected following partial purification from the cell lysate by binding to immobilized anti-Notch-1 antibody.

It is also envisioned to be within the scope of these methods of the invention that inhibition of Notch-1 gene expression can be detected by hybridizing a detectably labeled oligonucleotide probe specific for a portion of Notch-1 mRNA to nucleic acids under high stringency conditions, detecting hybridization of the labeled probe, and correlating hybridization with the level of Notch-1 mRNA. Alternatively, Notch-1 mRNA may be determined using quantitative RT-PCR. It is envisioned that Notch-1 mRNA quantitation may be conducted in a high-throughput system.

Inhibition of Notch-1 activity also can be detected by electrophoretically separating proteins in cell lysates and subjecting the proteins to Western blot analysis and probing the blots for the active, cytoplasmic form of Notch-1 (N^{IC}). Since N^{IC} translocates to the nucleus, nuclear extracts also can be probed for N^{IC} levels using Western blot analysis. However, as one of ordinary skill in the art will appreciate, a decrease in Notch-1 activity may be detected by any suitable means. It is specifically envisioned that inhibition of Notch-1 activity could be detected with or without first fractionating cellular proteins. For example, the methods described in the Examples may be adapted for use with cells grown and treated in a well (e.g., a well in a multi-well plate), followed by in-well lysis and detection of N^{IC} protein in a quantitative or semi-quantitative screening assay without prior purification of the N^{IC} protein. Optionally, N^{IC} may be detected following partial purification from the cell lysate by binding to immobilized anti-N^{IC} antibody. In addition, inhibition of Notch-1 activity can be detected by monitoring the activity of downstream targets, such as the transcription factor CBF-1, which in turn modulates the expression of multiple targets including several transcription factors. For example, the effect of inhibition of Notch-1 activity on these downstream effectors can be monitored using reporter gene constructs regulated by CBF-1, including but not limited to HES1, HES5, HEY1, HEY2, or any of the other downstream effectors of Notch-1. Such reporter gene constructs are well known in the art.

As one skilled in the art will appreciate, the methods of the invention can be used to screen any molecule or plurality of molecules for the ability to inhibit Notch-1 gene expression or activity, including but not limited to libraries of currently available molecules (i.e., gamma secretase inhibitor libraries or proteasome inhibitor libraries), libraries of modified proteasome inhibitors, and libraries of modified gamma secretase inhibitors. Furthermore, N-benzyloxycarbonyl-leucyl-leucyl-norleucinal derivatives (z-Leu-Leu-Nle-CHO) (FIG. 13A) can be tested for the ability to inhibit Notch-1 gene expression or activity in cancer cells. As used herein N-benzyloxycarbonyl-leucyl-leucyl-norleucinal derivatives includes related molecules derived from or synthesized to have a structure similar to N-benzyloxycarbonyl-leucyl-leucyl-norleucinal (z-Leu-Leu-Nle-CHO) (FIG. 13A) that can be tested for its ability to inhibit Notch-1 gene expression or activity. For example, suitable N-benzyloxycarbonyl-leucyl-leucyl-norleucinal derivatives would include molecules structurally similar to N-benzyloxycarbonyl-leucyl-leucyl-norleucinal in which the aldehyde group of N-benzyloxycarbonyl-leucyl-leucyl-norleucinal is replaced with boronate to create a peptidyl boronic acid or z-Leu-Leu-Nle-B(OH)₂ (FIG. 13B). It is well within the ability of one of ordinary skill in the art to obtain N-benzyloxycarbonyl-leucyl-leucyl-norleucinal or other gamma secretase inhibitor derivatives using any suitable synthesis. For example, one could obtain a N-benzyloxycarbonyl-leucyl-leucyl-norleucinal boronate derivative by according to methods described in described by Adams et al., 1998, Bioorganic and Medicinal Chemistry Lett. 8:333-338.

Other suitable N-benzyloxycarbonyl-leucyl-leucyl-norleucinal derivatives include those having a substituent at the N-terminus other than “z”. For example, it is specifically envisioned that the N-terminus could be modified to include any acyl group. In addition, it one or more of the hydrophobic amino acids of N-benzyloxycarbonyl-leucyl-leucyl-norleucinal or other peptide gamma secretase inhibitors may be replaced with another hydrophobic amino acid (e.g., leucine, isoleucine, valine, phenylalanine, tyrosine, alanine, methoinine, threonine, beta alanine) in any combination provided that each amino acid is different from at least one other amino acid comprising the molecule. Further, it is envisioned that one or more of the L-amino acids could be replaced with the corresponding D-amino acid or non-naturally-occurring amino acids (e.g., phenylalanine in which the aromatic ring is substituted with one or more fluorine atoms). In additional embodiments, the amino acids could be linked by one or more non-peptide bonds. Disulfide bonds may be added to stabilize longer peptides.

Preferably, inhibition of Notch-1 gene expression or activity caused by the compounds disclosed herein, or identified using the screening methods discloses herein, is accompanied by increased apoptosis in Notch-1 inhibited cells relative to apoptosis observed in untreated or control cells. In yet other preferred embodiments, Notch-1 inhibition caused by the compounds disclosed herein, or identified using the screening methods discloses herein, is accompanied by the ability to inhibit cell growth in cancer cells

In yet another aspect, the invention provides methods for inducing apoptosis in cancer cells comprising the step of contacting the cancer cells with an apoptosis-inducing effective amount of N-benzyloxycarbonyl-leucyl-leucyl-norleucinal.

The term “apoptosis-inducing effective amount” refers to the amount of a compound of the invention determined to produce any increase in the percentage of cells killed within a population of cells or any increase in the rate with which cancer cells are killed within a population of cells. As one of skill in the art will appreciate, benefit may be conferred without achieving 100% killing. Such apoptosis-inducing effective amounts are readily ascertained by one of ordinary skill in the art and using methods as described herein.

Cancer cells useful in said disclosed methods of inducing apoptosis in cancer cells are melanoma, multiple myeloma, prostate cancer, osteosarcoma, breast cancer, or cervical cancer cells.

In another aspect, the present invention provides methods for inhibiting cell growth in cancer cells comprising the step of contacting the cells with a growth inhibiting-effective amount of N-benzyloxycarbonyl-leucyl-leucyl-norleucinal.

The term “growth inhibiting-effective amount” refers to the amount of a compound of the invention determined to produce any inhibition in cell growth in cancer cells, such as cell cycle arrest. Such growth inhibiting-effective amounts are readily ascertained by one of ordinary skill in the art and using methods as described herein.

Cancer cells useful in the methods of inhibiting cell growth in cancer cells comprising the step of contacting the cells with a growth inhibiting-effective amount of N-benzyloxycarbonyl-leucyl-leucyl-norleucinal are melanoma, multiple myeloma, prostate cancer, osteosarcoma, breast cancer, or cervical cancer cells.

In further aspects, the invention provides methods for inducing apoptosis in melanoma cells, myeloma cells, prostate cancer cells, osteosarcoma, breast cancer or cervical cancer cells comprising the step of contacting the cells with an apoptosis-inducing effective amount of a gamma secretase inhibitor and a proteasome inhibitor.

In certain embodiments, a single dual inhibitor compound is used in the inventive methods for inducing apoptosis in melanoma cells, myeloma cells, prostate cancer cells, osteosarcoma, breast cancer or cervical cancer cells as disclosed herein. Non-limiting examples of dual inhibitor compounds for use in these methods of the invention are leucyl-leucyl-norleucinal, N-benzyloxycarbonyl-leucyl-leucyl-norleucinal and N-benzyloxycarbonyl-leucyl-leucyl-leucinal (also known as MG-132).

In certain other embodiments, the gamma secretase inhibitor and the proteasome inhibitor used in the methods of inducing apoptosis in melanoma cells, myeloma cells, prostate cancer cells, osteosarcoma, breast cancer or cervical cancer cells disclosed herein are separate and distinct compounds.

In further aspects, the invention provides methods for inhibiting cell growth in melanoma cells, myeloma cells, prostate cancer cells, osteosarcoma, breast cancer or cervical cancer cells comprising the step of contacting the cells with an growth-inhibiting effective amount of a gamma secretase inhibitor and a proteasome inhibitor.

In certain embodiments, a single dual inhibitor compound is used in the inventive methods for inhibiting cell growth in melanoma cells, myeloma cells, prostate cancer cells, osteosarcoma, breast cancer or cervical cancer cells as disclosed herein. Non-limiting examples of dual inhibitor compounds for use in these methods of the invention are leucyl-leucyl-norleucinal, N-benzyloxycarbonyl-leucyl-leucyl-norleucinal and N-benzyloxycarbonyl-leucyl-leucyl-leucinal (also known as MG-132).

In certain other embodiments, the gamma secretase inhibitor and the proteasome inhibitor used in the methods of inhibiting cell growth in melanoma cells, myeloma cells, prostate cancer cells, osteosarcoma, breast cancer or cervical cancer cells disclosed herein are separate and distinct compounds.

In other aspects, the invention provides methods for inducing apoptosis in melanoma cells, myeloma cells, prostate cancer cells, osteosarcoma, breast cancer or cervical cancer cells comprising the step of contacting the cells with an apoptosis-inducing effective amount of a gamma secretase inhibitor and a tumoricidal agent.

In certain embodiments, the gamma secretase inhibitor used in said method is N-benzyloxycarbonyl-leucyl-leucyl-norleucinal. In certain embodiments, the tumoricidal agent is a proteasome inhibitor, an AKT inhibitor, a mitotic poison, a DNA damaging agent, a growth factor receptor antagonist, or a STAT3 inhibitor. Particularly preferred tumoricidal agents are platinum coordination complexes, AKT inhibitor I, MG-132 and Paclitaxel.

In yet other aspects, the invention provides methods for inhibiting cell growth in melanoma cells, myeloma cells, prostate cancer cells, osteosarcoma, breast cancer or cervical cancer cells comprising the step of contacting the cells with a growth inhibiting-effective amount of a gamma secretase inhibitor and a tumoricidal agent.

In certain embodiments, the gamma secretase inhibitor used in these methods is N-benzyloxycarbonyl-leucyl-leucyl-norleucinal. In certain embodiments, the tumoricidal agent is a proteasome inhibitor, an AKT inhibitor, a mitotic poison, a DNA damaging agent, a growth factor receptor antagonist, or a STAT3 inhibitor. Particularly preferred tumoricidal agents are platinum coordination complexes, AKT inhibitor I, MG-132 and Paclitaxel.

In still further aspects, the invention provides methods for inducing apoptosis in breast cancer or cervical cancer cells comprising the step of contacting the cells with an apoptosis-inducing effective amount of a tumoricidal agent and a compound that inhibits Notch-1 gene expression or protein activity.

In certain embodiments, the tumoricidal agent used in these methods is a proteasome inhibitor, an AKT inhibitor, a mitotic poison, a DNA damaging agent, a growth factor receptor antagonist, or a STAT3 inhibitor. Particularly preferred tumoricidal agents are platinum coordination complexes, AKT inhibitor I, MG-132 and Paclitaxel. In the present method of inducing apoptosis in breast cancer or cervical cancer cells, any siRNA targeting the human Notch-1 gene can be used. In preferred embodiments, the compound that inhibits Notch-1 gene expression or protein activity is a Notch-1 siRNA, more particularly 5′-AAG TGT CTG AGG CCA GCA AGA-3′ (SEQ ID NO. 1).

In other aspects, the present invention provides methods for inhibiting cell growth in breast cancer or cervical cancer cells comprising the step of contacting the cells with a growth inhibiting effective amount of a tumoricidal agent and a compound that inhibits Notch-1 gene expression or protein activity.

In certain embodiments, the tumoricidal agent used in these methods is a proteasome inhibitor, an AKT inhibitor, a mitotic poison, a DNA damaging agent, a growth factor receptor antagonist, or a STAT3 inhibitor. Particularly preferred tumoricidal agents are platinum coordination complexes, AKT inhibitor I, MG-132 and Paclitaxel. In the present method of inhibiting cell growth in breast cancer or cervical cancer cells, any siRNA targeting the human Notch-1 gene can be used. In preferred embodiments, the compound that inhibits Notch-1 gene expression or protein activity is a Notch-1 siRNA, more particularly 5′-AAG TGT CTG AGG CCA GCA AGA-3′ (SEQ ID NO. 1).

In yet other aspect, the methods of the invention utilize a compound that induces apoptosis in a cancer cell having the formula:
Z-(AA)ₙ-Y

wherein
each AA is independently a hydrophobic L- or D-amino acid, a non-naturally occurring amino acid, a sulfonamide or a benzodiazepine, provided that at least one amino acid is different from at least one other amino acid;
Z is an acyl group or is absent;
Y is an aldehyde group, a boronate group, an α-keto acid, an α-keto ester, an α-keto amide, an epoxyketone, a vinyl sulfone, an α-keto heterocycle or is absent; and
n is an integer from 3 to 5 when AA is an amino acid and n is 1 when AA is a sulfonamide or a benzodiazepine.

In a preferred embodiment, the Z in a compound of the formula Z-(AA)ₙ-Y is benzyloxycarbonyl. In a particularly preferred embodiment, the compound of the formula Z-(AA)ₙ-Y is N-benzyloxycarbonyl-leucyl-leucyl-norleucinal. In one embodiment, a compound of the formula Z-(AA)ₙ-Y contains one or a plurality of the amino acids covalently linked by a non-peptide bond.

As used herein, the twenty conventional amino acids and their abbreviations follow conventional usage. See IMMUNOLOGY—A SYNTHESIS, 2nd Edition, (E. S. Golub and D. R. Gren, Eds.), 1991, Sinauer Associates, Sunderland, Mass., which is incorporated herein by reference for any purpose.

Naturally occurring residues may be divided into classes based on common side chain properties: 1) hydrophobic: norleucine, Met, Ala, Val, Leu, Ile; 2) neutral hydrophilic: Cys, Ser, Thr, Asn, Gln; 3) acidic: Asp, Glu; 4) basic: His, Lys, Arg; 5) residues that influence chain orientation: Gly, Pro; and 6) aromatic: Trp, Tyr, Phe.

Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties. In contrast, non-conservative substitutions may involve the exchange of a member of one of these classes for a member from another class.

In making such changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5) (Kyte et al., 1982, J Mol. Biol. 157:105-131).

The importance of the hydropathic amino acid index in conferring interactive biological function on a protein is understood in the art (see, for example, Kyte et al., 1982, ibid.). It is known that certain amino acids may be substituted for other amino acids having a similar hydropathic index or score and still retain a similar biological activity. It is also understood in the art that the substitution of like amino acids can be made effectively on the basis of hydrophilicity.

As described in U.S. Pat. No. 4,554,101, the following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4).

Exemplary amino acid substitutions are set forth in Table 1.

TABLE 1
Amino Acid Substitutions

| Original | Exemplary | Preferred |
|---|---|---|
| Residues | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, Gln, Asn, | Arg |
| | 1,4 Diamine-butyric | |
| | Acid | |
| Met | Leu, Phe, Ile | Leu |
| Phe | Leu, Val, Ile, Ala, | Leu |
| | Tyr | |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

Preferred amino acids in the compound of the invention include leucine, isoleucine, valine, phenylalanine, tyrosine, alanine, methoinine, threonine and beta alanine.

One skilled in the art may generate test variants containing a single amino acid substitution at each amino acid residue. The variants can then be screened using activity assays known to those skilled in the art and described herein. Such variants can be used to gather information about suitable variants. For example, if it was discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other substitutions. Alternatively, it is within the skill of one having skill in the art to synthesize any combination of 3-, 4- or 5-amino acid containing embodiments of the formula set forth above and test the resulting molecules individually or batch-wise to identify active compounds.

Stereoisomers (e.g., D-amino acids) of the twenty conventional amino acids, non-naturally occurring amino acids such as α-,α-disubstituted amino acids, N-alkyl amino acids, lactic acid, and other unconventional amino acids may also be suitable components for compounds of the present invention. Examples of unconventional amino acids include but are not limited to: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, 1-N-methylarginine, and other similar amino acids and imino acids (e.g., 4-hydroxyproline). One or more of the amino acid side chains may be modified to include fluorine. In the polypeptide notation used herein, the left-hand direction is the amino terminal direction and the right-hand direction is the carboxy-terminal direction, in accordance with standard usage and convention.

Peptide analogs are commonly used in the pharmaceutical industry as non-peptide drugs with properties analogous to those of the template peptide. These types of non-peptide compound are termed “peptide mimetics” or “peptidomimetics.” (See Fauchere, 1986, Adv. Drug Res. 15: 29; Veber and Freidinger, 1985, TINS p. 392; and Evans et al., 1987, J. Med. Chem. 30: 1229, which are incorporated herein by reference for any purpose.) Such compounds are often developed with the aid of computerized molecular modeling. Peptide mimetics that are structurally similar to therapeutically useful peptides may be used to produce a similar therapeutic or prophylactic effect. Generally, peptidomimetics are structurally similar to a paradigm polypeptide (i.e., a polypeptide that has a biochemical property or pharmacological activity), but have one or more peptide linkages optionally replaced by a linkage such as: —CH₂NH—, —CH₂S—, —CH₂—CH₂—, —CH═CH—(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used in certain embodiments to generate more stable peptides. In addition, conformationally-constrained peptides comprising a consensus sequence or a substantially identical consensus sequence variation may be generated by methods known in the art (Rizo and Gierasch, 1992, Ann. Rev. Biochem. 61: 387), incorporated herein by reference for any purpose); for example, by adding internal cysteine residues capable of forming intramolecular disulfide bridges which cyclize the peptide. In addition to peptides, other molecules that can be used as chemical scaffolds to synthesize compounds of the invention include, without limitation, sulfonamides and benzodiazepines.

In certain aspects, the invention provides pharmaceutical compositions that induce apoptosis in cancer cells comprising a gamma secretase inhibitor and a proteasome inhibitor in combination and a pharmaceutically-acceptable carrier, excipient, diluent or adjuvant.

In other aspects, the invention provides pharmaceutical compositions that induce apoptosis in cancer cells comprising a gamma secretase inhibitor and a tumoricidal agent in combination and a pharmaceutically-acceptable carrier, excipient, diluent or adjuvant.

In yet other aspects, the invention provides pharmaceutical compositions that induce apoptosis in cancer cells comprising a compound that inhibits Notch-1 gene expression or protein activity and a tumoricidal agent in combination and a pharmaceutically-acceptable carrier, excipient, diluent or adjuvant. In the present pharmaceutical compositions that induce apoptosis in cancer cells, any compound that inhibits Notch-1 gene expression or protein activity can be used. In preferred embodiments, the compound that inhibits Notch-1 gene expression or protein activity is a Notch-1 siRNA, more particularly 5′-AAG TGT CTG AGG CCA GCA AGA-3′ (SEQ ID NO. 1).

In yet further aspects, the invention provides pharmaceutical compositions that inhibit cell growth in cancer cells comprising a gamma secretase inhibitor and a proteasome inhibitor in combination and a pharmaceutically-acceptable carrier, excipient, diluent or adjuvant.

In still further aspects, the invention provides pharmaceutical compositions that inhibit cell growth in cancer cells comprising a gamma secretase inhibitor and a tumoricidal agent in combination and a pharmaceutically-acceptable carrier, excipient, diluent or adjuvant.

In additional aspects, the invention provides pharmaceutical compositions that inhibit cell growth in cancer cells comprising a compound that inhibits Notch-1 gene expression or protein activity and a tumoricidal agent in combination and a pharmaceutically-acceptable carrier, excipient, diluent or adjuvant. In the present pharmaceutical compositions that inhibit cell growth in cancer cells, any compound that inhibits Notch-1 gene expression or protein activity can be used. In preferred embodiments, the compound that inhibits Notch-1 gene expression or protein activity is a Notch-1 siRNA, more particularly 5′-AAG TGT CTG AGG CCA GCA AGA-3′ (SEQ ID NO. 1).

The term “pharmaceutical composition” as used herein refers to a composition comprising a pharmaceutically acceptable carrier, excipient, diluent or adjuvant, and a chemical compound, peptide, or composition as described herein that is capable of inducing a desired therapeutic effect when properly administered to an animal.

The pharmaceutical compositions of the invention may be delivered to cancer cells or tumors comprised thereof by any suitable mode of administration. For example, depending on the type and location of the cancer modes of administration of the pharmaceutical compositions of the invention include orally, through injection by intravenous, intraperitoneal, intracerebral (intra-parenchymal), intracerebroventricular, intramuscular, intra-ocular, intraarterial, intraportal, or intralesional routes; subcutaneously; topically; transdermally; rectally; vaginally; nasally; ocularly; by sustained release systems; by implantation devices; by liposomes; by micelles; or by depots formulations. The pharmaceutical compositions may be administered by bolus injection or continuously by infusion, or by implantation device. The pharmaceutical composition also can be administered locally via implantation of a membrane, sponge or another appropriate material onto which the desired molecule has been absorbed or encapsulated. Where an implantation device is used, the device may be implanted into any suitable tissue or organ, and delivery of the desired molecule may be via diffusion, timed-release bolus, or continuous administration.

Acceptable formulation materials preferably are nontoxic to recipients at the dosages and concentrations employed. The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20 and polysorbate 80, Triton, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, REMINGTON'S PHARMACEUTICAL SCIENCES, 18^{th} Edition, (A. R. Gennaro, ed.), 1990, Mack Publishing Company.

Optimal pharmaceutical compositions can be determined by one skilled in the art depending upon, for example, the intended route of administration, delivery format and desired dosage. See, for example, REMINGTON'S PHARMACEUTICAL SCIENCES, Id. Such compositions may influence the physical state, stability, rate of in vivo release and rate of in vivo clearance of the antibodies of the invention.

The primary vehicle or carrier in a pharmaceutical composition may be either aqueous or non-aqueous in nature. Por example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Pharmaceutical compositions can comprise Tris buffer of about pH 7.0-8.5, or acetate buffer of about pH 4.0-5.5, which may further include sorbitol or a suitable substitute therefor. Pharmaceutical compositions of the invention may be prepared for storage by mixing the selected composition having the desired degree of purity with optional formulation agents (REMINGTON'S PHARMACEUTICAL SCIENCES, Id.) in the form of a lyophilized cake or an aqueous solution.

Formulation components are present in concentrations that are acceptable to the site of administration. Buffers are advantageously used to maintain the composition at physiological pH or at a slightly lower pH, typically within a pH range of from about 5 to about 8.

The pharmaceutical compositions of the invention can be delivered parenterally. When parenteral administration is contemplated, the therapeutic compositions for use in this invention may be in the form of a pyrogen-free, parenterally acceptable aqueous solution comprising the desired compound identified in a screening method of the invention in a pharmaceutically acceptable vehicle. A particularly suitable vehicle for parenteral injection is sterile distilled water in which the compound identified in a screening method of the invention is formulated as a sterile, isotonic solution, appropriately preserved. Preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which may then be delivered via a depot injection. Formulation with hyaluronic acid has the effect of promoting sustained duration in the circulation. Implantable drug delivery devices may be used to introduce the desired molecule.

The compositions may be formulated for inhalation. In these embodiments, a compound of the invention is formulated as a dry powder for inhalation, or inhalation solutions may also be formulated with a propellant for aerosol delivery, such as by nebulization. Pulmonary administration is further described in PCT Application No. PCT/US94/001875, which describes pulmonary delivery of chemically modified proteins and is incorporated by reference.

The pharmaceutical compositions of the invention can be delivered through the digestive tract, such as orally. The compounds of the invention that are administered in this fashion may be formulated with or without those carriers customarily used in the compounding of solid dosage forms such as tablets and capsules. The pharmaceutical compositions of the invention may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations.

Tablets contain the active ingredient in admixture with non-toxic pharmaceutically acceptable excipients which are suitable for the manufacture of tablets. These excipients may be for example, inert diluents, such as calcium carbonate, sodium carbonate or bicarbonate, lactose, calcium phosphate or sodium phosphate; granulating and disintegrating agents, for example, microcrystalline cellulose, sodium croscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropyl-cellulose, or a time delay material such as ethyl cellulose, cellulose acetate buryrate may be employed. By dissolving the tablets in sterile water, or another appropriate vehicle, solutions may be prepared in unit-dose form.

A capsule may be designed to release the active portion of the formulation at the point in the gastrointestinal tract when bioavailability is maximized and pre-systemic degradation is minimized. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil. Additional agents can be included to facilitate absorption of the compound of the invention. Diluents, flavorings, low melting point waxes, vegetable oils, lubricants, suspending agents, tablet disintegrating agents, and binders may also be employed.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

Compounds of the invention may also be administered in the form of suppositories for rectal administration. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing a compound of the invention are employed. (For purposes of this application, topical application shall include mouth washes and gargles.)

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

Additional pharmaceutical compositions are evident to those skilled in the art, including formulations involving a compound of the invention in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, PCT Application No. PCT/US93/00829, which describes the controlled release of porous polymeric microparticles for the delivery of pharmaceutical compositions. Sustained-release preparations may include semipermeable polymer matrices in the form of shaped articles, e.g. films, or microcapsules, polyesters, hydrogels, polylactides (U.S. Pat. No. 3,773,919 and EP 058,481), copolymers of L-glutamic acid and gamma ethyl-L-glutamate (Sidman et al., 1983, Biopolymers 22: 547-556), poly (2-hydroxyethyl-methacrylate) (Langer et al., 1981, J. Biomed. Mater. Res. 15: 167-277) and Langer, 1982, Chem. Tech. 12: 98-105), ethylene vinyl acetate (Langer et al., id.) or poly-D(−)-3-hydroxybutyric acid (EP 133,988). Sustained release compositions may also include liposomes, which can be prepared by any of several methods known in the art. See e.g., Eppstein et al., 1985, Proc. Natl. Acad. Sci. USA 82: 3688-3692; EP 036,676; EP 088,046 and EP 143,949.

The pharmaceutical composition to be used for in vivo administration typically is sterile. In certain embodiments, this may be accomplished by filtration through sterile filtration membranes. In certain embodiments, where the composition is lyophilized, sterilization using this method may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

The effective amount of a pharmaceutical composition of the invention to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which the pharmaceutical composition is being used, the route of administration, and the size (body weight, body surface or organ size) and/or condition (the age and general health) of the patient. A clinician may titer the dosage and modify the route of administration to obtain the optimal therapeutic effect. Typical dosages range from about 0.1 μg/kg to up to about 100 mg/kg or more, depending on the factors mentioned above. In certain embodiments, the dosage may range from 0.1 μg/kg up to about 100 mg/kg; or 1 μg/kg up to about 100 mg/kg; or 5 μg/kg up to about 100 mg/kg.

The dosing frequency will depend upon the pharmacokinetic parameters of a compound of the invention in the composition. For example, a clinician administers the composition until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

The invention also provides methods for treating an animal that has cancer, particularly melanoma, prostate cancer, multiple myeloma, osteosarcoma, breast cancer or cervical cancer comprising the step of administering a therapeutically-effective amount of a pharmaceutical composition of the invention.

The term “therapeutically effective amount” refers to the amount of a pharmaceutical composition of the invention or a compound identified in a screening method of the invention determined to produce a therapeutic response in an animal. Such therapeutically effective amounts are readily ascertained by one of ordinary skill in the art and using methods as described herein.

As used herein, the term “treating” in the context of treating an animal that has cancer refers to an activity that prevents, alleviates or ameliorates any of the primary phenomena (initiation, progression, metastasis) or secondary symptoms associated with the disease.

In certain embodiments, these methods for treating an animal that has cancer, particularly melanoma, prostate cancer, multiple myeloma or osteosarcoma, comprises the step of administering a therapeutically-effective amount of a pharmaceutical composition that induces apoptosis in cancer cells comprising a gamma secretase inhibitor and a proteasome inhibitor in combination and a pharmaceutically-acceptable carrier, excipient, diluent or adjuvant.

In other embodiments, these method for treating an animal that has cancer, particularly melanoma, prostate cancer, multiple myeloma or osteosarcoma, comprises the step of administering a therapeutically-effective amount of a pharmaceutical composition that induces apoptosis in cancer cells comprising a gamma secretase inhibitor and a tumoricidal agent in combination and a pharmaceutically-acceptable carrier, excipient, diluent or adjuvant.

In yet other embodiments, these methods of treating an animal that has cancer, particularly melanoma, prostate cancer, multiple myeloma or osteosarcoma, comprises the step of administering a therapeutically-effective amount of a pharmaceutical composition that inhibits cell growth in cancer cells comprising a gamma secretase inhibitor and a proteasome inhibitor in combination and a pharmaceutically-acceptable carrier, excipient, diluent or adjuvant.

In additional embodiments, these method of treating an animal that has cancer, particularly melanoma, prostate cancer, multiple myeloma or osteosarcoma, comprises the step of administering a therapeutically-effective amount of a pharmaceutical composition that inhibits cell growth in cancer cells comprising a gamma secretase inhibitor and a tumoricidal agent in combination and a pharmaceutically-acceptable carrier, excipient, diluent or adjuvant.

In certain embodiments, these methods for treating an animal that has cancer, particularly melanoma, prostate cancer, multiple myeloma or osteosarcoma, comprises the step of administering a therapeutically-effective amount of a pharmaceutical composition that induces apoptosis in cancer cells comprising a compound that inhibits Notch-1 gene expression or protein activity and a tumoricidal agent in combination and a pharmaceutically-acceptable carrier, excipient, diluent or adjuvant. The present pharmaceutical compositions that induces apoptosis in cancer cells can comprise any compound that inhibits Notch-1 gene expression or protein activity. In preferred embodiments, the compound that inhibits Notch-1 gene expression or protein activity is a Notch-1 siRNA, more particularly 5′-AAG TGT CTG AGG CCA GCA AGA-3′ (SEQ ID NO. 1).

In certain embodiments, these methods for treating an animal that has cancer, particularly melanoma, prostate cancer, multiple myeloma or osteosarcoma, comprises the step of administering a therapeutically-effective amount of a pharmaceutical composition that inhibits cell growth in cancer cells comprising a compound that inhibits Notch-1 gene expression or protein activity and a tumoricidal agent in combination and a pharmaceutically-acceptable carrier, excipient, diluent or adjuvant. The present pharmaceutical compositions that inhibit cell growth in cancer cells can comprise any compound that inhibits Notch-1 gene expression or protein activity. In preferred embodiments, the compound that inhibits Notch-1 gene expression or protein activity is a Notch-1 siRNA, more particularly 5′-AAG TGT CTG AGG CCA GCA AGA-3′ (SEQ ID NO. 1).

In yet further aspects, the invention provides methods for treating an animal that has cancer, particularly breast cancer or cervical cancer, comprising the step of administering a therapeutically-effective amount of a pharmaceutical composition of the invention

In certain embodiments, these methods for treating an animal that has cancer, particularly breast cancer or cervical cancer, comprises the step of administering a therapeutically-effective amount of a pharmaceutical composition that induces apoptosis in cancer cells comprising a gamma secretase inhibitor and a proteasome inhibitor in combination and a pharmaceutically-acceptable carrier, excipient, diluent or adjuvant.

In certain embodiments, these methods for treating an animal that has cancer, particularly breast cancer or cervical cancer, comprises the step of administering a therapeutically-effective amount of a pharmaceutical composition that induces apoptosis in cancer cells comprising a gamma secretase inhibitor and a tumoricidal agent in combination and a pharmaceutically-acceptable carrier, excipient, diluent or adjuvant.

In certain embodiments, these methods for treating an animal that has cancer, particularly breast cancer or cervical cancer, comprises the step of administering a therapeutically-effective amount of a pharmaceutical composition that induces apoptosis in cancer cells comprising a compound that inhibits Notch-1 gene expression or protein activity and a tumoricidal agent in combination and a pharmaceutically-acceptable carrier, excipient, diluent or adjuvant. The present pharmaceutical compositions that induces apoptosis in cancer cells can comprise any compound that inhibits Notch-1 gene expression or protein activity. In preferred embodiments, the compound that inhibits Notch-1 gene expression or protein activity is a Notch-1 siRNA, more particularly 5′-AAG TGT CTG AGG CCA GCA AGA-3′ (SEQ ID NO. 1).

In certain embodiments, these methods for treating an animal that has cancer, particularly breast cancer or cervical cancer, comprises the step of administering a therapeutically-effective amount of a pharmaceutical composition that inhibits cell growth in cancer cells comprising a gamma secretase inhibitor and a proteasome inhibitor in combination and a pharmaceutically-acceptable carrier, excipient, diluent or adjuvant.

In certain embodiments, these methods for treating an animal that has cancer, particularly breast cancer or cervical cancer, comprises the step of administering a therapeutically-effective amount of a pharmaceutical composition that inhibits cell growth in cancer cells comprising a gamma secretase inhibitor and a tumoricidal agent in combination and a pharmaceutically-acceptable carrier, excipient, diluent or adjuvant.

In certain embodiments, these methods for treating an animal that has cancer, particularly breast cancer or cervical cancer, comprises the step of administering a therapeutically-effective amount of a pharmaceutical composition that inhibits cell growth in cancer cells comprising a compound that inhibits Notch-1 gene expression or protein activity and a tumoricidal agent in combination and a pharmaceutically-acceptable carrier, excipient, diluent or adjuvant. The present pharmaceutical compositions that inhibit cell growth in cancer cells can comprise any compound that inhibits Notch-1 gene expression or protein activity. In preferred embodiments, the compound that inhibits Notch-1 gene expression or protein activity is a Notch-1 siRNA, more particularly 5′-AAG TGT CTG AGG CCA GCA AGA-3′ (SEQ ID NO. 1).

As utilized in accordance with the present disclosure, the following terms, unless otherwise indicated, shall be understood to have the following meanings:

The terms “polypeptide” or “protein” is used herein to refer to native proteins, that is, proteins produced by naturally-occurring and specifically non-recombinant cells, or by genetically-engineered or recombinant cells, and comprise molecules having the amino acid sequence of the native protein, or sequences that have deletions, additions, and/or substitutions of one or more amino acids of the native sequence.

The term “naturally-occurring” as used herein refers to an object that can be found in nature, for example, a polypeptide or polynucleotide sequence that is present in an organism (including a virus) that can be isolated from a source in nature and which has not been intentionally modified by man; conversely, “non-naturally occurring” does not encompass these embodiments. The term “naturally occurring” or “native” when used in connection with biological materials such as nucleic acid molecules, polypeptides, host cells, and the like, refers to materials which are found in nature and are not manipulated by man. Similarly, “recombinant,” “non-naturally occurring” or “non-native” as used herein refers to a material that is not found in nature or that has been structurally modified or synthesized by man.

The term “vector” is used to refer to any molecule (e.g., nucleic acid, plasmid, or virus) used to transfer coding information to a host cell or a target cell.

The term “agent” is used herein to denote a chemical compound, a mixture of chemical compounds, a biological macromolecule, or an extract made from biological materials.

It is well within the ability of one skilled in the art to establish suitable dose ranges for any particular compound of the invention. Suitable doses for inducing apoptosis, inhibiting cell growth, inducing NOXA and inhibiting Notch-1 expression or activity in cancer cells in an animal may depend on a variety of factors, including the age, size, and physical condition of the animal (e.g., hepatic or renal function), the activity and toxicity of the molecule and its metabolites, the half life of the molecule in the body, the type of cancer cell, the stage of cancer, and location of the cancer.

Conventional techniques well known to those with skill in the art were used for recombinant DNA production, oligonucleotide synthesis, and tissue culture and cell transformation (e.g., electroporation, lipofection) procedures. Enzymatic reactions and purification techniques were performed according to manufacturers' specifications or as commonly accomplished in the art or as described herein. The techniques and procedures were generally performed according to conventional methods well known in the art and as described in various general and more specific references that are cited and discussed throughout the present specification. See e.g., Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), which are incorporated herein by reference for any purpose. Unless specific definitions are provided, the nomenclature utilized in connection with, and the laboratory procedures and techniques of, molecular biology, genetic engineering, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

All references cited are herein incorporated by reference in their entirety.

As used herein, the singular forms “a”, “an” and “the” include plural referents unless the context clearly dictates otherwise.

### EXAMPLES

The following non-limiting Examples are intended to be purely illustrative.

### Example 1

### Effect of z-Leu-Leu-Nle-CHO on Melanoma Cells and Normal Human Melanocytes In Vitro

The effect of z-Leu-Leu-Nle-CHO on apoptosis was tested in vitro using normal human melanocytes and several melanoma cell cultures. Prior to testing, cutaneous (C8161, SK-MeI-28, SK-MeI-100, SK-MeI-5, C81-61), uveal or ocular (MUM2B, MUM2C, OCM-1A), and pulmonary metastases (RJ002L) melanoma cell lines were maintained in RPMI supplemented with 10% FBS. Normal human melanocytes were isolated from neonatal foreskins as previously described (Qin J et al., 2004, Mol Cancer Ther 3:895-902) and cultured with medium 154 containing growth supplements (Cascade Biologics, Portland, Oreg.).

The p53 gene of the melanoma cell lines was characterized. First, DNA was isolated from each cell line using standard methods. Then, each exon of the p53 gene was amplified using PCR, and the PCR products were purified using solid phase reversible immobilization (SPR1)-based technology (AMPURE; Agencourt Biosciences Corp., Beverly, Mass.). Sequencing reactions were performed using BigDye Terminator v3.1 premix on GeneAmp 9700 PCR machines (Applied Biosystems, Foster City, Calif.). Sequencing reactions were purified using CLEANSEQ (Agencourt Biosciences Corp., Beverly, Mass.) and analyzed on 3730×1 DNA analyzers (Applied Biosystems, Foster City, Calif.).

To assess the effect of z-Leu-Leu-Nle-CHO on the cell lines, the cells were grown in suitable culture media. Highly proliferating cells (i.e., cells that had reached approximately 30% confluency) were exposed to z-Leu-Leu-Nle-CHO as described below. The culture media was replenished with media containing z-Leu-Leu-Nle-CHO at various concentrations and the cells were incubated for a specified period of time prior to harvesting the cells for further analysis.

Following treatment with z-Leu-Leu-Nle-CHO, the melanoma and melanocyte cell cultures were assayed for apoptotic cells using the APO TARGET Annexin V FITC staining kit (Biosource, Camarillo, Calif.) according to the manufacturer's instructions followed by flow cytometric analysis using FACScaliber (Becton Dickinson, Palo Alto, Calif.) as described in Qin, J. et al., 2001, Nat Med 7:385-386. In some cases, cell cycle and apoptosis analysis was measured using propidium iodide staining and flow cytometry as described. Cells with DNA content less than the G₀ amount of untreated cells were considered apoptotic. For cell analysis, DNA histograms were analyzed using MultiCycle for Windows (Phoenix Flow Systems, San Diego, Calif.) as described in Denning, M. F. et al., 1998, J Biol Chem 273: 29995-30002.

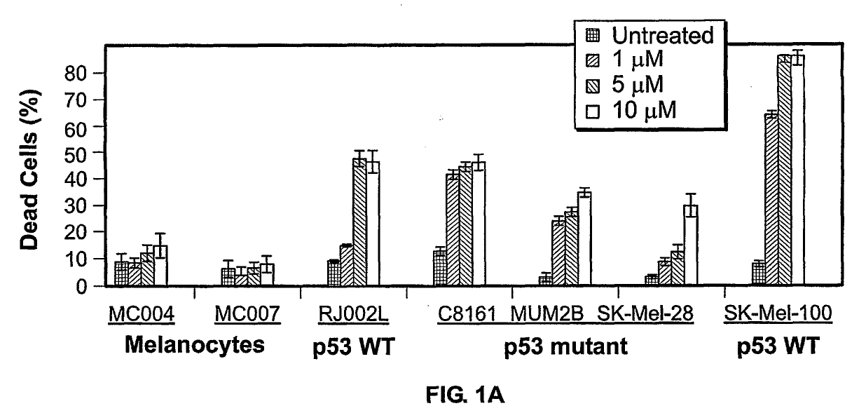

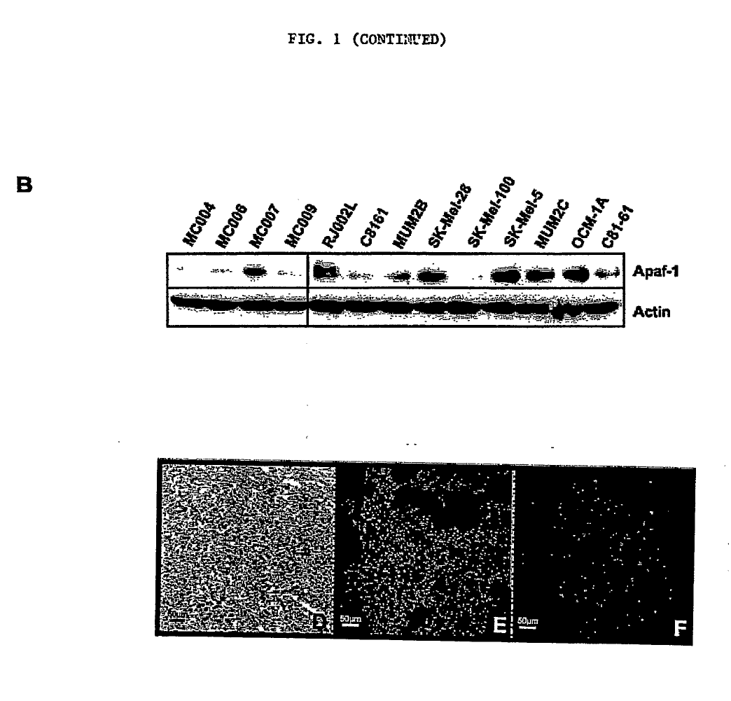

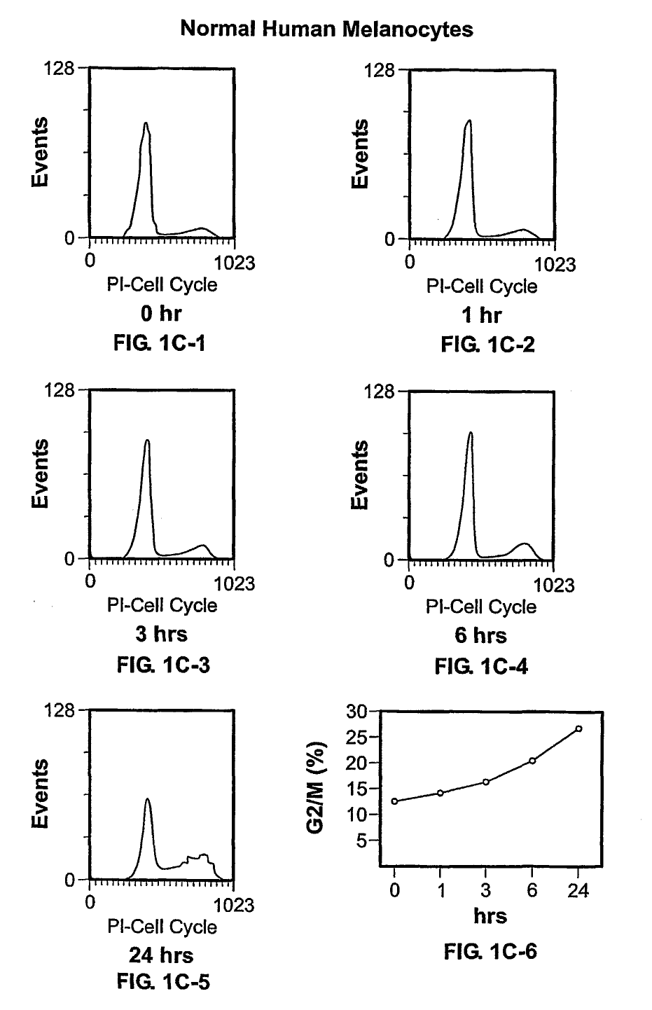

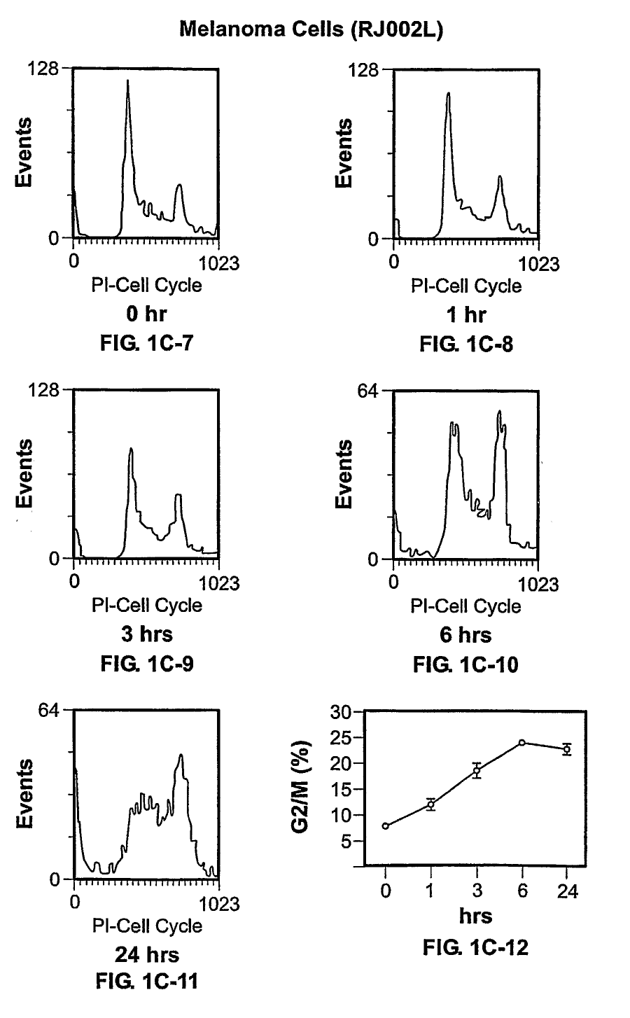

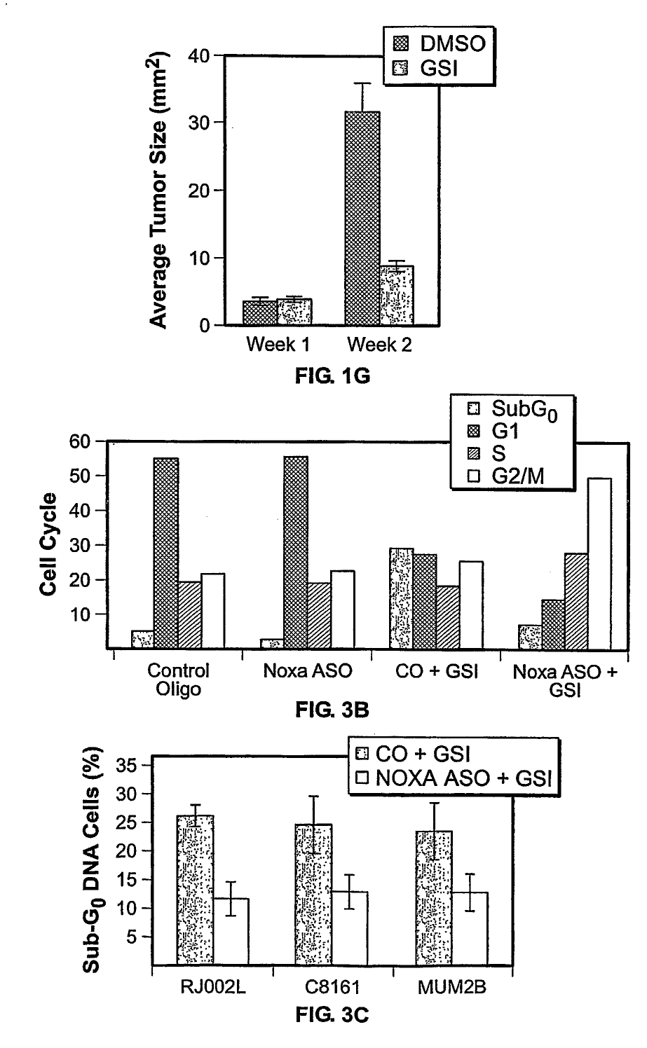

The addition of z-Leu-Leu-Nle-CHO to all cultures of proliferating normal human melanocytes (MC004, MC007) did not significantly induce apoptosis (FIG. 1A; 1-10 μM for 18 h), but it did induce a G2/M growth arrest (FIG. 1C; 10 μM for 24 h). In contrast, in each of the nine tested melanoma cell lines, z-Leu-Leu-Nle-CHO (10 μM for 24 h) triggered a dose-dependent increase in apoptosis, which was preceded by a G2/M growth arrest (see FIG. 1C for representative results from RJ002L melanoma cells). FIG. 1A shows representative apoptotic results for five different melanoma cell lines (RJ002L and SK-MeI-100, which possess wild-type p53; and C8161, MUM2B, and SK-MeI-28, each of which possess a p53 mutation) before and 18 hrs after contacting the cells with z-Leu-Leu-Nle-CHO (1-10 μM).

Because epigenetic inactivation of Apaf-1 was reported to occur in approximately 50% of melanoma cell lines, Apaf-1 levels in each of the melanoma cell lines and normal human melanocytes were measured by Western blot analysis. Whole cell extracts were prepared and analyzed as described by Denning, M. F. et al., 1998, J Biol Chem 273. 29995-30002, 1998. The enriched mitochondria pellet and mitochondria-free cytosol of melanoma cells were prepared with the APO ALERT cell fraction kit (Clontech Laboratories, Inc., Palo Alto, Calif.) according to the manufacturer's instructions. Equal amounts of protein extracted from each sample (20-30 μg) were loaded into pre-cast 7% NuPAGE Tris-acetate gels or 4-12% NuPAGE Bis-tris gels (Invitrogen). Proteins were separated by SDS-PAGE and transferred to polyvinylidene difluoride membranes (Bio-Rad, Hercules, Calif.) using Invitrogen's buffering system. The indicated primary antibodies were incubated for indicated period of time, washed, and visualized by incubation with horseradish peroxidase-conjugated secondary antibodies and chemiluminescent reagents (Roche Diagnostics Corporation, Indianapolis, Ind.). Antibodies to Apaf-1 (559683-BD Bioscience) were used for the analysis. Secondary antibodies anti-goat, anti-mouse, anti-rabbit and anti-rat (IgG-HRP) were purchased from Vector Laboratories (Burlingame, Calif.) and Santa Cruz Biotechnology. As shown in FIG. 1B, higher Apaf-1 levels were observed in most melanoma lines, relative to Apaf-1 levels of melanocytes, although two melanoma lines (C8161 and SK-MeI-100) have relatively low Apaf-1 levels.

### Example 2

### Effect of z-Leu-Leu-Nle-CHO on Melanoma Cells In Vivo

Assessment of tumor formation in Nude mice was performed by adapting the method described in Hendrix, M. J. et al., 1997, Am J Pathol 150:483-495. Tumors were established in Nude mice as follows. Briefly, 1×10⁶ aggressive melanoma cells (C8161) (ATCC, Rockville, Md.) were injected subcutaneously in Nude mice on day 0, and the tumors were allowed to grow for 1 week. Beginning on day 8, mice received injections (100 μl) of DMSO carrier only (control group) or z-Leu-Leu-Nle-CHO (1 mM in DMSO) (Calbiochem, LaJolla, Calif.) (experimental group) every other day for 1 week. Tumor sizes (mm²) were determined on day 7 prior to injections with or z-Leu-Leu-Nle-CHO or DMSO, and on day 14. Average tumor size for both groups was normalized, and size of tumors at 2 weeks was averaged. Detection of apoptosis in tissue sections was performed using TUNEL staining as described in Wrone-Smith et al, 1997, Am J Pathol. 151:1321-9.

FIGS. 1D-1F are images of z-Leu-Leu-Nle-CHO and DMSO treated melanoma cells to show z-Leu-Leu-Nle-CHO-induced apoptosis in melanoma cells. The cells are visualized by TUNEL staining and light microscopy. FIG. 1D shows Hematoxylin and Eosin (H&E) stained frozen section of the highly aggressive human cutaneous melanoma tumor xenograft (C8161 cells) after treatment with z-Leu-Leu-Nle-CHO (1 mM). High levels of apoptosis are observed by immunofluorescent TUNEL assay following 1 week of treatment with z-Leu-Leu-Nle-CHO (1 mM) (FIG. 1E) versus DMSO carrier (FIG. 1F). The H&E and TUNEL staining were performed as described in Wrone-Smith et al, 1997, Am J Pathol. 151(5):1321-9. FIG. 1G is a graph of data showing the average tumor size prior to treatment (week 1) for control (DMSO) and z-Leu-Leu-Nle-CHO-treated (“GSI”) mice and the average tumor size after one week of treatment (week 2) for control (DMSO) and z-Leu-Leu-Nle-CHO-treated (“GSI”) mice. Reduction in average tumor size following z-Leu-Leu-Nle-CHO administration was significant as z-Leu-Leu-Nle-CHO treatment for 1 week significantly reduced tumor size (p=0.046) compared to DMSO (n=11).

### Example 3

### Effectiveness of z-Leu-Leu-Nle-CHO Versus Chemotherapeutic Agents on Killing Melanoma Cells In Vitro

An initial dosing study comparing z-Leu-Leu-Nle-CHO against three different chemotherapeutic agents (i.e., adriamycin, etoposide, and cisplatin) was performed to evaluate the relative effectiveness of z-Leu-Leu-Nle-CHO in killing melanoma cells. In agreement with a previous report by Soengas, M. S. et al., 2001, Nature 409.207-211, SK-MeI-28 cells, carrying a mutated p53, were relatively resistant to killing by adriamycin, as well as to killing by other chemotherapeutic agents. For SK-MeI-28 melanoma cells, the maximal apoptotic response for adriamycin, etoposide, cisplatin, and z-Leu-Leu-Nle-CHO was 12.0%, 2.6%, 4.0%, and 29.4%, respectively. For RJ002L melanoma cells, the maximal apoptotic response for adriamycin, etoposide, cisplatin, and z-Leu-Leu-Nle-CHO was 23.9%, 12.7%, 3.4%, and 45.7%, respectively. Thus, z-Leu-Leu-Nle-CHO was more effective in killing both SK-MeI-28 and RJ002L melanoma cells than any of the tested chemotherapeutic agents.

### Example 4

### Effect of z-Leu-Leu-Nle-CHO on pro-Apoptotic Proteins Including NOXA in Melanoma Cells

To assess the effect of z-Leu-Leu-Nle-CHO on pro-apoptotic proteins, RJ002L pulmonary metastases melanoma cells were grown in RPMI supplemented with 10% FBS. Highly proliferating cells (i.e., cells that had reached approximately 30% confluency) were exposed to z-Leu-Leu-Nle-CHO by replenishing the culture media with media containing z-Leu-Leu-Nle-CHO (10 μM) (experimental group) or media without z-Leu-Leu-Nle-CHO (control group) and the cells were incubated for a specified period of time prior to harvesting the cells for further analysis.

Western blot analyses following z-Leu-Leu-Nle-CHO and control treatment of RJ002L melanoma cells were performed to determine the effect of z-Leu-Leu-Nle-CHO on pro-survival and pro-apoptotic proteins. Whole cell extracts were prepared and analyzed as described by Denning, M. F. et al., 1998, J Biol Chem 273: 29995-30002, 1998. The enriched mitochondria pellet and mitochondria-free cytosol of melanoma cells were prepared with the APO ALERT cell fraction kit (Clontech Laboratories, Inc. Palo Alto, Calif.) according to the manufacturer's instructions. For Western blot analysis, equal amounts of protein extracted from each sample (20-30 μg) were loaded into pre-cast 7% NuPAGE Tris-acetate gels or 4-12% NuPAGE Bis-tris gels (Invitrogen). Proteins were separated by SDS-PAGE and transferred to polyvinylidene difluoride membranes (Bio-Rad, Hercules, Calif.) using Invitrogen's buffering system. The indicated primary antibodies were incubated for indicated period of time, washed, and visualized by incubation with horseradish peroxidase-conjugated secondary antibodies and chemiluminescent reagents (Roche Diagnostics Corporation, Indianapolis, Ind.). Antibodies used were as follows: From Santa Cruz Biotechnology: Bcl-2 (SC-7382), BCl-x_{L} (SC-634), Mcl-1 (SC-819), survivin (SC-10811), Bax (SC-493), Bak (SC-832), Bim (SC-11425), PUMA (SC-19187), p53 (SC-126), and GADD45 (SC-796); From Cell Signaling: Bid (#2002), and cleaved caspase 9 (9501); NOXA (OP180—Oncogene Research Products), Apaf-1 (559683-BD Bioscience), Smac/Diablo (IMG-248-Imgenex), cytochrome C (part of kit—Apo Alert™ cell fraction kit—Clontech Laboratories, Inc.—Palo Alto, Calif.), PARP (Pharmingen, San Diego, Calif.) and β-actin (Sigma Chemical Co., St. Louis, Mo.). Secondary antibodies anti-goat, anti-mouse, anti-rabbit and anti-rat (IgG-HRP) were purchased from Vector Laboratories (Burlingame, Calif.), Santa Cruz Biotechnology and Amersham Biosciences.

FIG. 2A shows the profile of pro-survival and pro-apoptotic proteins in RJ002L melanoma cells following z-Leu-Leu-Nle-CHO exposure (10 μM; left side) compared to medium alone (right side). As seen in FIG. 2A, z-Leu-Leu-Nle-CHO treatment of RJ002L melanoma cells induced expression of Bak, Bim, and NOXA, whereas neither PUMA nor Bax levels appeared to be affected by z-Leu-Leu-Nle-CHO treatment. NOXA induction was detectable as early as 1 hr and was massive by 6-18 hrs. z-Leu-Leu-Nle-CHO treatment of normal melanocytes did not induce any of the pro-apoptotic proteins (i.e., Bak, Bim, or NOXA) (data not shown). Induction by z-Leu-Leu-Nle-CHO of these pro-apoptotic proteins in melanoma cell lines coincided with onset of apoptosis, Bid degradation, and the appearance of cleaved caspase 9 and PARP. At 6 hrs, there was little to no decrease in the levels of survival proteins (Bcl-2, Bcl-x_{L}, Mcl-1, or survivin). However, at longer time intervals (i.e., 18 hrs and 24 hrs), decreases in Bcl-2 and survivin levels were observed in the melanoma cells treated with z-Leu-Leu-Nle-CHO. Interestingly, Western blot analysis at similar time points for several melanoma cell lines following exposure to DNA damaging agents such as adriamycin, etoposide and cisplatin revealed that the tested DNA damaging agents did not induce NOXA (data not shown).

Mitochondrial integrity before and after exposure to z-Leu-Leu-Nle-CHO was evaluated using Western blot analysis as described above. As shown in FIG. 2B, cytochrome C and Smac/DLABLO were released into the cytoplasm of RJ002L melanoma cells as early as 1 hr following z-Leu-Leu-Nle-CHO exposure (10 μM).

Because NOXA is prominently induced in z-Leu-Leu-Nle-CHO-treated melanoma cells, but not in z-Leu-Leu-Nle-CHO-treated melanocytes, further investigations into the role of NOXA in z-Leu-Leu-Nle-CHO-induced apoptosis were undertaken. To determine if p53 status influences induction of NOXA, NOXA induction in z-Leu-Leu-Nle-CHO-treated melanoma cell lines (10 μM) containing p53 mutations (i.e. C8161, SK-MeI-28, MUM2B) were examined using Western blot analysis as described above and compared to that of Western blot results from z-Leu-Leu-Nle-CHO-treated melanoma cell lines (10 μM) having wild-type p53 (RJ002L, SK-MeI-100). As shown in FIG. 2C and, in relevant part, FIG. 2A, little to no p53 was detected in MUM2B and SK-MeI-28, which carry homozygous inactivating mutations (R196Stop and L145R, respectively), either before or after z-Leu-Leu-Nle-CHO exposure. In contrast, C8161, which carries a R196Stop mutation in only one allele, has p53 expression similar to that observed in wild-type cell lines. Induction of NOXA in response to z-Leu-Leu-Nle-CHO was observed in all melanocytes, regardless of p53 expression or mutation, indicating that NOXA induction is independent of p53. Since SK-MeI-100 cells, which have wild-type p53 and low Apaf-1 levels (FIG. 1B), were found to be highly sensitive to z-Leu-Leu-Nle-CHO-mediated killing (FIG. 1A), Apaf-1 and NOXA levels were examined before and after z-Leu-Leu-Nle-CHO treatment (10 μM) using Western blot analysis as described above. NOXA induction occurred earlier in SK-MeI-100 cells than in the other tested cell lines (FIG. 2C). Apaf-1 levels were not increased in response to z-Leu-Leu-Nle-CHO (data not shown).

Further evidence that p53 does not appear to be either necessary or sufficient for induction of NOXA was obtained by evaluating the effect of siRNA directed against p53 on NOXA induction in z-Leu-Leu-Nle-CHO-treated RJ002L cells. Smart pools of p53 siRNA duplexes were purchased from Upstate Biotechnology (Charlottesville Va.). Scramble control duplex was obtained from Dharmacon (LaFayette, Colo.). Melanoma cells were plated in 6-well plates at a density of 1.5×10⁵ cells per well. siRNA duplexes were transfected with oligofectamine in Opti-MEM medium (InVitrogen), using the manufacturer's protocol. Forty-eight hrs later, transfected cells were treated with z-Leu-Leu-Nle-CHO (10 μM) for another 24 hrs before assaying by Western blot analysis as described above. Despite a significant reduction in p53 levels (>80% reduction), NOXA was induced by z-Leu-Leu-Nle-CHO (FIG. 2D). The activity of p53 siRNA was confirmed by reduced induction of GADD45 and MDM2, which are p53-inducible proteins (FIG. 2D).

In summary, z-Leu-Leu-Nle-CHO induced NOXA in all tested melanoma cell lines, regardless of p53 mutation status, with lines carrying mutated p53 exhibiting delayed NOXA expression in response to z-Leu-Leu-Nle-CHO (FIGS. 2A and 2C). In addition, while all normal melanocytes contained abundant p53 levels, z-Leu-Leu-Nle-CHO did not induce NOXA in normal melanocytes (data not shown). Thus, within these two different cell types (i.e., melanocytes and melanoma cells), p53 does not appear to be either necessary or sufficient for induction of NOXA.

The role of NOXA in z-Leu-Leu-Nle-CHO-mediated apoptosis of melanoma cells was further evaluated using RJ002L, C8161 and MUM2B melanoma cell lines containing antisense oligonucleotide targeting NOXA. The antisense oligonucleotides (ASO) included a NOXA targeted sequence (5′-TCA GTC TAC TGA TTT ACT GG-3′) (SEQ ID NO: 2) and a control oligonucleotide (CO) (5′-CCT TCC CTG AAG GTT CCT CC-3′) (SEQ ID NO: 3). The ASO were purchased from ISIS Pharmaceuticals Inc. (Carlsbad, Calif.) as previously described by Qin J et al., 2004, Mol Cancer Ther 3:895-902. Melanoma cells were seeded at 2×10⁵ cells into 6 well plates I day before transfection. Opti-MEM (InVitrogen) was pre-incubated for 30 minutes at room temperature using a ratio of 3 μM/mL lipofectamine per 100 nmol/L to produce a final oligonucleotide concentration of 50 nmol/L. Cells were washed with PBS and transfection mix (1 ml) was added. After 4 hrs of incubation, RPMI 1640 (1 mL) containing 20% fetal bovine serum and z-Leu-Leu-Nle-CHO (10 μM) was added to the cells and the cells were incubated for 24 hrs. The treated cells were analyzed for cell viability using APO TARGET Annexin V-FITC staining kits (Biosource), for cell cycle progression and by Western blot using NOXA, Bim, Bak and β-actin antibodies as described above.

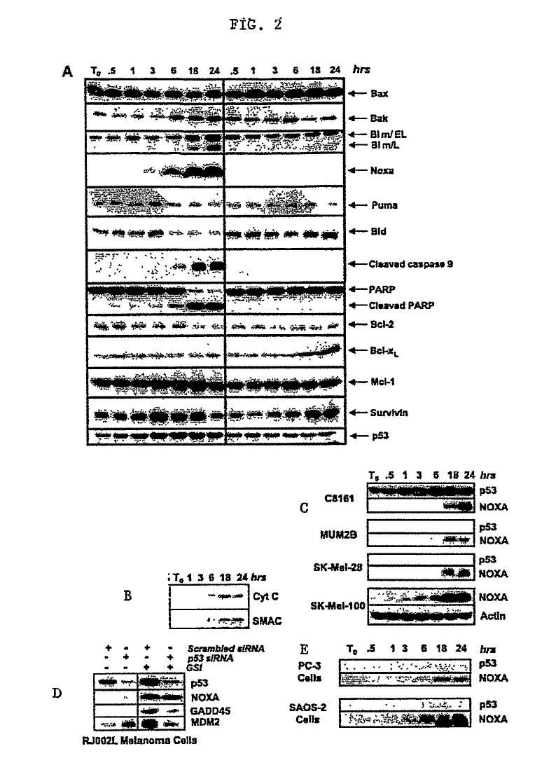

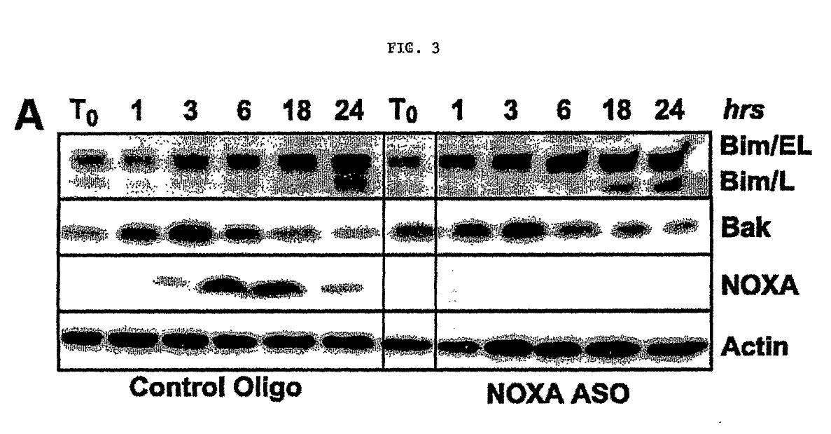

Pre-incubation of RJ002L melanoma cells with the anti-NOXA antisense oligonucleotide (ASO), but not control oligonucleotide (CO), blocked induction of NOXA, but not Bim or Bak (FIG. 3A, left panel). The ability of the anti-NOXA ASO to block NOXA induction (FIG. 3A, right panel) was correlated with a significant reduction in the apoptotic response (FIGS. 3B and 3C). Interestingly, z-Leu-Leu-Nle-CHO treatment of melanoma cells in the absence of NOXA induction was correlated with an increase in the relative percentage of melanoma cells in G2/M (FIG. 3B), which resembles the response of normal melanocytes to z-Leu-Leu-Nle-CHO (compare FIG. 3B with FIG. 1C). While neither the NOXA ASO nor the CO alone influenced the cell cycle or spontaneous apoptosis for RJ002L, C8161, or MUM2B cells, the NOXA ASO significantly reduced z-Leu-Leu-Nle-CHO mediated apoptosis (determined using sub-G0 DNA content) observed in 3-4 independent experiments for each tested cell line (p<0.01 for all cell lines comparing CO+z-Leu-Leu-Nle-CHO versus NOXA ASO+z-Leu-Leu-Nle-CHO) (FIG. 3C).

A role for caspase activation in z-Leu-Leu-Nle-CHO-mediated apoptosis was confirmed by pretreating RJ002L cells with various membrane permeable inhibitors against a broad group of caspases (i.e., ZVAD-CHO), as well as more selective inhibitors targeting caspase 9 (LETD-CHO) or and caspase 3 (DEVD-CHO) (caspases obtained from Calbiochem, San Diego, Calif.). Each of these caspase inhibitors reduced the z-Leu-Leu-Nle-CHO-mediated apoptotic response in melanoma cells by approximately 30-50% (data not shown).

### Example 5

### Effect of z-Leu-Leu-Nle-CHO on NOXA in Prostate Cancer and Osteosarcoma Cells

Two different cell lines known to be p53 null, PC-3 prostate cells and SAOS-2 osteosarcoma cells (ATCC, Rockville, Md.) were tested for z-Leu-Leu-Nle-CHO-induced expression of NOXA. Western blot analysis, performed as described above using p53 and NOXA antibodies described above, showed that z-Leu-Leu-Nle-CHO (10 μM) induced expression of NOXA (FIG. 2E) with prominent apoptotic responses (>50% dead cells after 24 hrs; data not shown), suggesting that the mechanism is not be limited to melanoma cell lines.

### Example 6

### Effect of Proteasome Inhibitors on Melanoma Cells and Normal Human Melanocytes In Vitro

The proteasome inhibitors lactacystin, MG-132, and bortezomib were evaluated for the ability to induce apoptosis in malignant melanoma cell lines (n=3) including an early passage line, RJ002L. MG-132 was obtained from Calbiochem (La Jolla, Calif.) and dissolved in DMSO. Lactacystin was purchased from Sigma Chemical Co. (St. Louis, Mo.) and dissolved in PBS. Bortezomib was obtained from a pharmacy as VELCADE (bortezomib) for Injection from a single use vial for intravenous use only (Millennium Pharmaceuticals, Cambridge, Mass.). A cell line with wild-type p53 (RJ002L) and two melanoma cell lines with mutant p53 alleles (C8161 and MUM2B) were included in this study. Two additional melanoma cell lines were also used: cutaneous SK-MeI-28 cells and primary, early passage MG012 cells. In addition, five normal melanocyte culture cell lines (MC-005, MC-006, MC-008, MC-009 and MC-011) were examined. Prior to testing, the melanoma cell lines were maintained in RPMI supplemented with 10% FBS. Normal human melanocytes were isolated from neonatal foreskins as previously described (Qin J et al., 2004, Mol Cancer Ther 3:895-902, 2004) and cultured with medium 154 containing growth supplements (Cascade Biologics, Portland, Oreg.).

To assess the effect of proteasome inhibitors on the cell lines, the cells were grown in suitable culture media as described above. Highly proliferating cells (i.e., cells that had reached approximately 30% confluency) were exposed to the inhibitors as described below. The culture media was replenished with media containing proteasome inhibitors at various concentrations and the cells were incubated for 24 hours prior to harvesting the cells for further analysis.

Following treatment with the proteasome inhibitors, the melanoma and melanocyte cell cultures were assayed for apoptotic cells using the APO TARGET Annexin V FITC staining kit (Biosource, Camarillo, Calif.) according to the manufacturer's instructions followed by flow cytometric analysis using FACScaliber (Becton Dickinson, Palo Alto, Calif.) as described in Qin, J. et al., 2001, Nat Med 7: 385-386. In some cases, cell cycle and apoptosis analysis was measured using propidium iodide staining and flow cytometry as described. Cells with DNA content less than the G0 amount of untreated cells were considered apoptotic. For cell analysis, DNA histograms were analyzed using MultiCycle for Windows (Phoenix Flow Systems, San Diego, Calif.) as described in Denning, M. F. et al., 1998, J Biol Chem 273: 29995-30002.

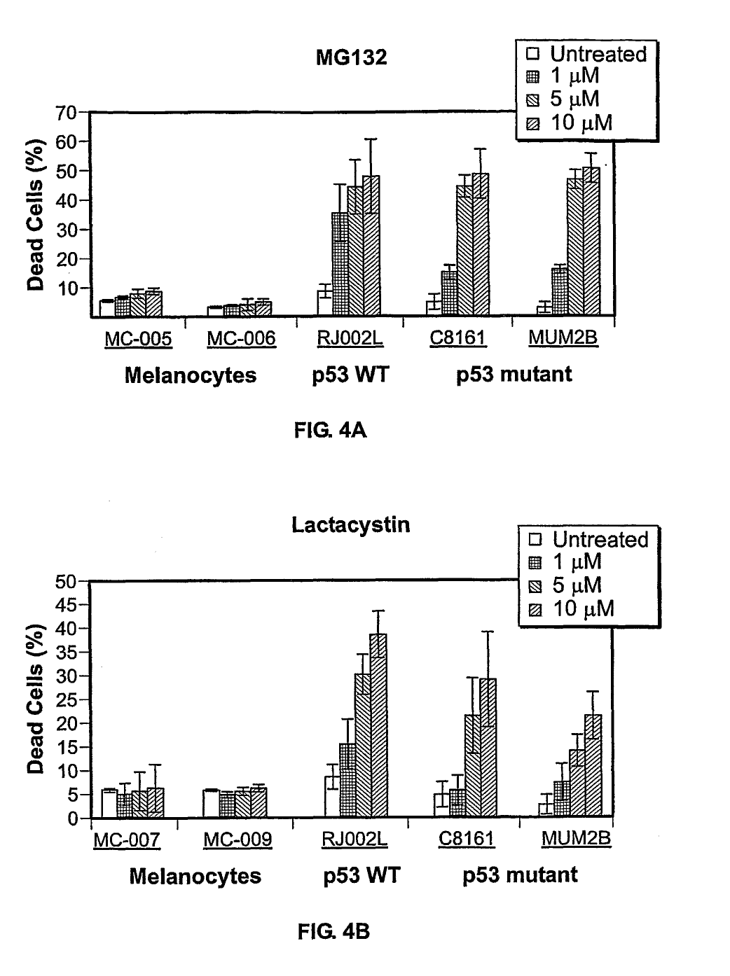

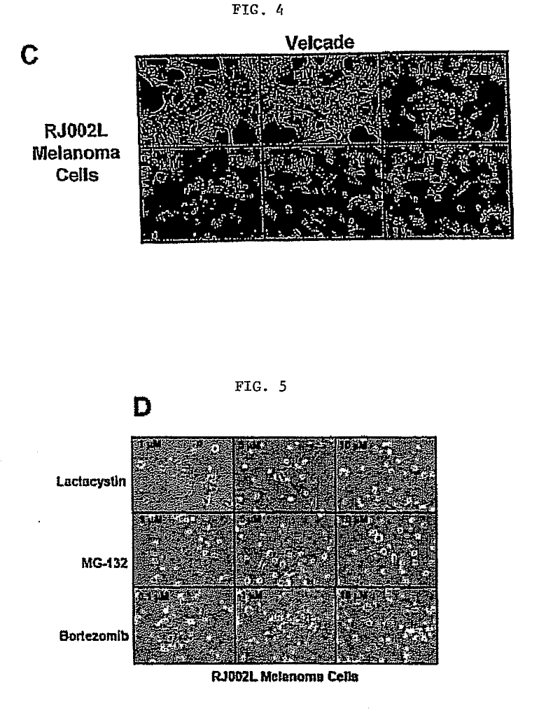

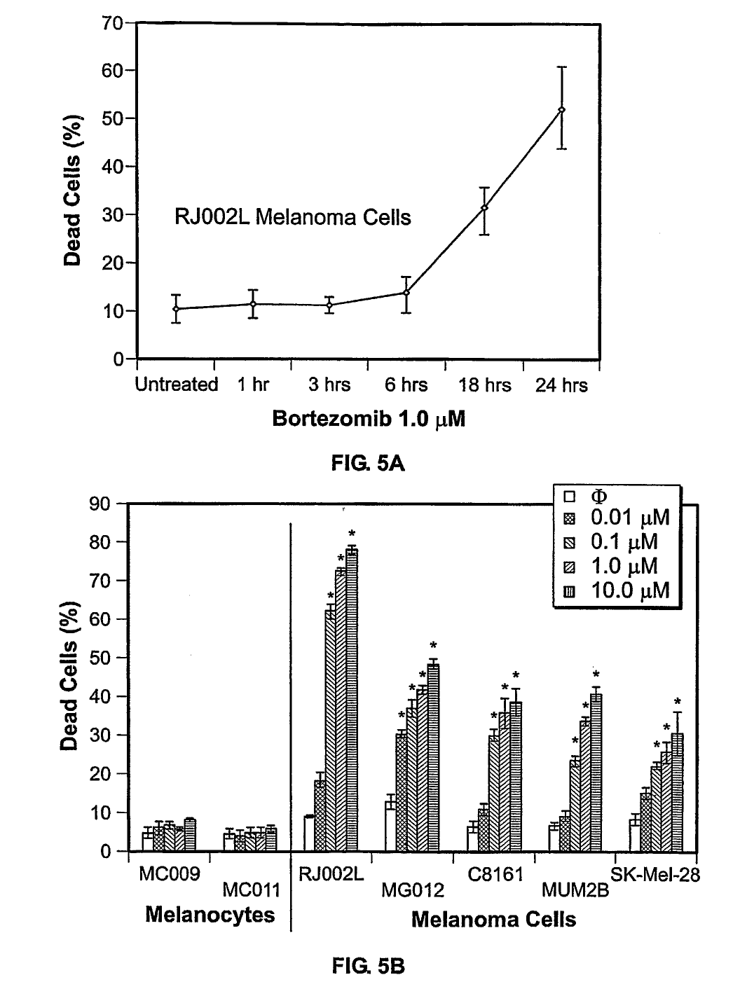

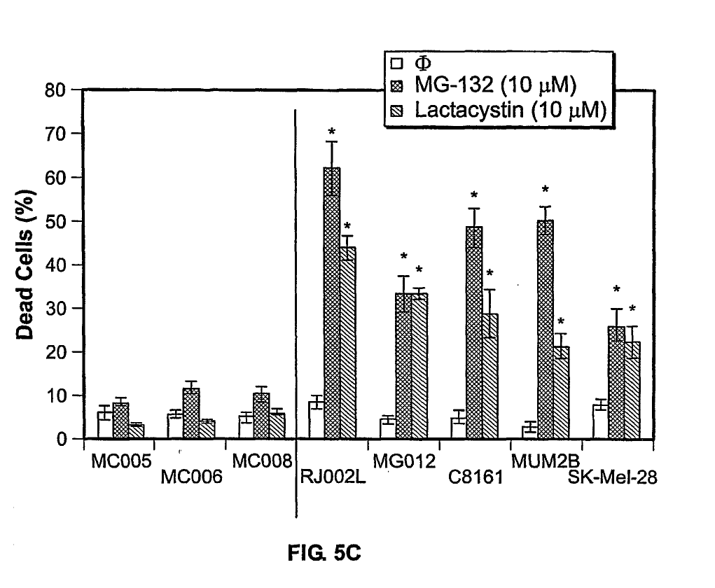

FIGS. 4A and 5C show that MG-132 (1-10 μM, FIG. 4A; 10 μM, FIG. 5C) increases cell death in melanoma cells but not in melanocytes, independent of p53 and in a concentration dependent manner. No more than 10% of normal melanocyte cultures designated as MC-005, MC-006 or MC-008 undergo apoptosis 24 hrs after exposure to MG-132 at concentrations of 1, 5 or 10 μM. In contrast, melanoma cell lines RJ002L, MG012, C8161, MUM2B and SK-MeI-28 undergo significantly enhanced apoptotic responses to increasing concentrations of MG-132, irrespective of their p53 status.

Similarly, lactacystin (1-10 μM, FIG. 4B; 10 μM, FIG. 5C) increases cell death in melanoma cells, even in MUM2B, which has little or p53, but did not cause cell death in melanocytes. As shown in FIGS. 4B and 5C, when the melanocytes were exposed to lactacystin, no more than 10% of normal melanocyte cultures designated as MC-005, MC-006 or MC-008 undergo apoptosis 24 hrs after exposure to lactacystin at concentrations of 1, 5 or 10 μM. In contrast, melanoma cell lines RJ002L, MG012, C8161, MUM2B and SK-MeI-28 undergo significantly enhanced apoptotic responses to increasing concentrations of lactacystin, irrespective of their p53 status. However, the degree of apoptosis was greater for RJ002L cells, which have wild type p53, than apoptosis for MUM2B cells, which possesses a p53 mutation. The C8161 cells, which carry a R196Stop mutation in only one allele, exhibited a percent cell death that is intermediate between wild-type cells and cells lacking p53 entirely. This suggests that lactacystin activates both p53-independent and -dependent pathways.

The ability of bortezomib to induce apoptosis in RJ002L cells was assayed by kinetic analysis. RJ002L cells were incubated with 1.0 μM bortezomib for 24 hours. At various time points during the 24 hours, the cells were assayed for apoptosis using APO TARGET Annexin V-FITC staining kits (Biosource, Camerillo, Calif.) according to manufacturer's instructions. The relative percentage of cells undergoing apoptosis was quantified by flow cytometric analysis using FACSCaliber (Becton Dickinson, Palo Alto, Calif.) as described in Qin J et al., 2004, Mol Cancer Ther 3:895-902. As shown in FIG. 5A, during the initial 6 hrs of exposure, minimal changes in viability of the culture were observed, but prominent apoptosis was detected at 18- and 24-hr time points.

As shown in FIG. 5B, bortezomib (0.01 to 10 μM) triggered an apoptotic response in less than 10% of all proliferating melanocytes (MC009 and MC011) after 24 hrs of continuous exposure (FIG. 5B, left side panel). In contrast, bortezomib triggered a dose-dependant increase in apoptosis of all proliferating melanoma cell lines tested ranging from 30-70% dead cells at a 10 μM concentration of the proteasome inhibitor after 24 hrs of continuous exposure (FIG. 5B, right side panel). For all melanoma cell lines examined with bortezomib, exposure to bortezomib at concentrations of 0.1 μM or greater triggered a significant apoptotic responses (p<0.05).

As can be seen from FIGS. 4C and 5D, which show images of RJ002L melanoma cells viewed with phase contrast microscopy before and after 24 hours of treatment with bortezomib (0.01 to 10 μM; FIG. 4C and 0.01 to 10 μM; FIG. 5D), lactacystin (1 to 10 μM; FIG. 5D) and MG-132 (1 to 10 μM; FIG. 5D) at varying concentrations, all three proteasome inhibitors induce apoptosis in melanoma cells in a concentration dependent manner. For example, FIG. 4C shows that while no apoptotic response is observed at a concentration of 0.01 μM bortezomib (top right panel) (2% of cells with sub-G0 DNA content by FACS analysis), increasing the concentration to 0.1 μM (bottom left panel) triggered a 10-fold increase (20%) in apoptotic cells, which was further increased to 29-30% using 1 μM (bottom middle panel) or 10 μM (bottom right panel). Melanoma cells exposed to concentrations of bortezomib ≧1 μM appeared rounded-up, with membrane blebbing and detachment from the dish.

### Example 7

### Effect of Bortezomib on Melanoma Cells In Vivo

A xenograft animal model system as described in Hendrix, M. J. et al., 1997, Am J Pathol 150:483-495 was utilized. Initially, 1×10⁶ aggressive melanoma cells (C8161) (ATCC, Rockville, Md.) were injected subcutaneously in Nude (nulnu) mice (6-7 weeks old; Harlan, Indianapolis, Ind.) on day 0, and the tumors were allowed bearing groups (5 mice/group) and injected with either (a) PBS as control; (b) bortezomib at 1.25 mg/kg; or (c) bortezomib at 2.5 mg/kg. Treatment began on day 8 when tumors were palpable and injected four times peri-tumorally. On day 20, mice were euthanized and tumors dissected from surrounding tissue and weighted. Detection of apoptosis in tissue sections was performed using Hematoxylin and Eosin staining as described in Wrone-Smith et al, 1997, Am J. Pathol. 151:1321-9.

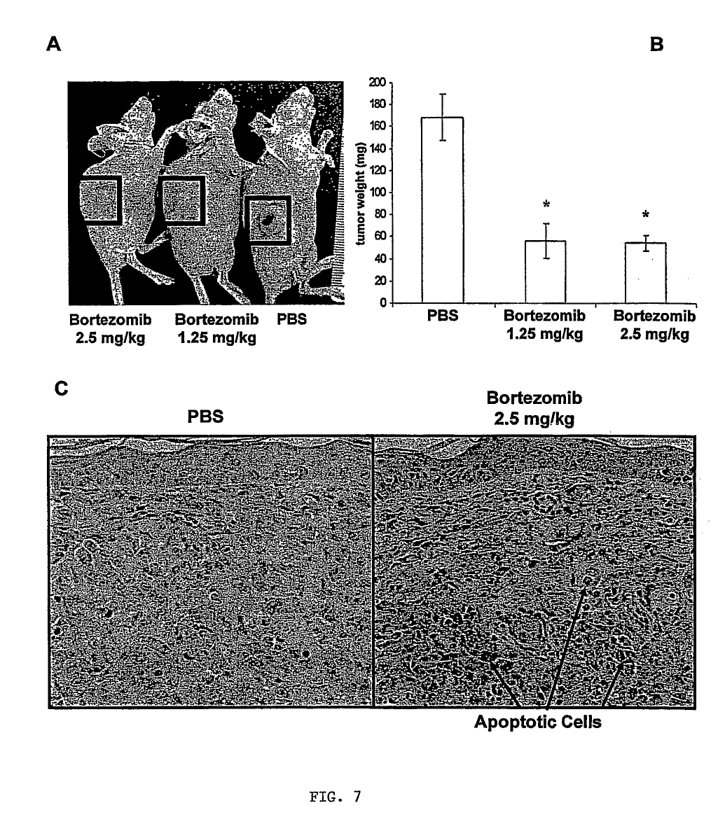

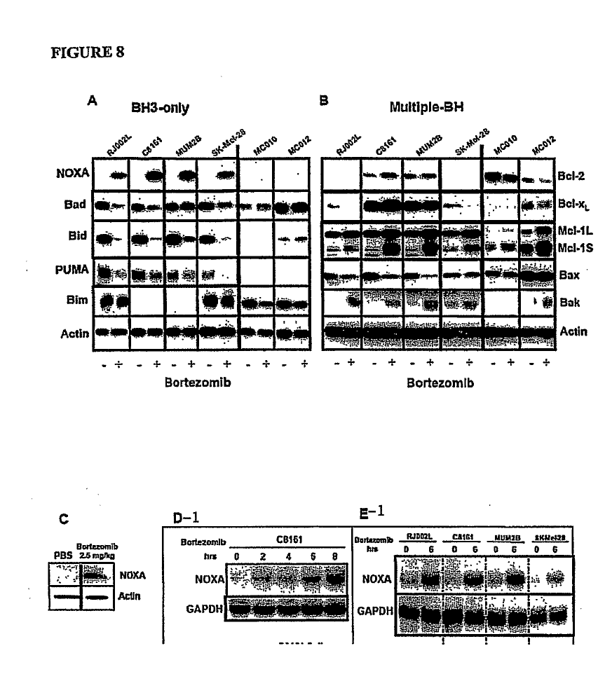

Compared to PBS-injected tumors that continued to grow, regression of melanoma tumors occurred using with 1.25 mg/kg or 2.5 mg/kg of bortezomib (FIGS. 7A and 7B). There was a significant (p<0.01) reduction in tumor weight comparing PBS injected versus tumors injected with 1.25 mg/kg bortezomib, and no further reduction was apparent at the 2.5 mg/kg dose (FIG. 7B).

PBS and bortezomib treated tumors were examined histologically using Hematoxylin and Eosin stain. Histological appearance of the tumors reveals an expansile mass of viable C8161 melanoma cells filling the upper dermis in tumor injected with PBS (FIG. 7C, left panel), compared to tumor injected with bortezomib at 2.5 mg/kg in which scattered apoptotic C8161 cells are present (FIG. 7C, right panel). The arrows in FIG. 7C indicate tumor cells undergoing apoptosis.

### Example 8

### Effect of Proteasome Inhibitors on Pro-Apoptotic Proteins Including NOXA in Melanoma Cells

A panel of melanoma cells were used to assess the effect of bortezomib on pro-apoptotic proteins. RJ002L, C8161, MUM2B and SK-MeI-28 melanoma cells, as well as two normal melanocyte cultures (MC-010 and MC-012) were examined. Prior to testing, the RJ002L melanoma cell lines were maintained in RPMI supplemented with 10% FBS. Late passage melanoma cell lines (C8161, MUM2B and SK-Me1028) were utilized as previously described (Welch D et al., 1991, Int J Cancer 47:227-237; Bittner M et al., 2000, Nature 406:536-540). Normal human melanocytes were isolated from neonatal foreskins and cultured as previously described (Qin J et al., 2004, Mol Cancer Ther 3:895-902). Proliferating cells were examined by Western blot analysis before and after 18 hrs of exposure to bortezomib (1 μM). Antibodies were obtained as follows: Bad (SC-8044), Bcl-2 (SC-7382), Bcl-x_{L} (SC-634), Mcl-1 (SC-819), Bak (SC-832) from Santa Cruz Biotechnology (Santa Cruz, Calif.); NOXA (OP180) from Calbiochem; Bid, Bim and PUMA from Cell Signaling (Beverly, Mass.); BAX from Upstate (Charlottesville, Va.); and β-actin from ICN (Irvine, Calif.). For the Western blot analysis, whole cell extracts were prepared as previously described (Denning M et al., 1998, J Biol Chem 273:29995-30002). Briefly, cells were harvested by scraping monolayers and washed with PBS. Cell pellets were resuspended in CHAPS buffers containing a protease inhibitor cocktail. Extracts were vigorously shaken at 4° C. for 15 min followed by centrifugation. Supernatants were collected and protein concentration determined using Bio-Rad reagent. Thirty to 50 microgram protein samples were resolved by SDS-polyacrylamide gel electrophoresis and transferred to polyvinylidene difluoride membrane by electroblotting. Membranes were probed with the various primary antibodies overnight at 4° C., followed by detection using ECL reagents (Amersham Pharmacia Biotech, Piscataway, N.J.) according to manufacturer's instructions.

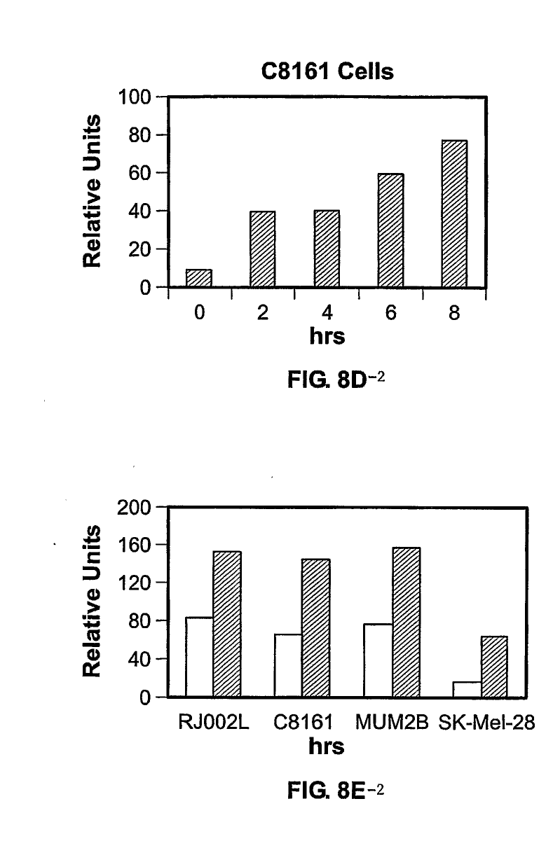

FIGS. 8A and 8B show the profile of B1H3-only proteins (FIG. 8A) and multiple BH-related proteins (FIG. 8A) in the melanoma and melanocytes cells following bortezomib exposure. Among the five different BH3-only family members examined, only NOXA was consistently induced in all four melanoma cells by bortezomib (FIG. 8A). Other BH3-only proteins examined in these melanoma cell lines revealed constitutive levels of Bad, Bid, PUMA and Bim. After bortezomib exposure, Bad, Bid and PUMA levels decreased, with no changes in Bim levels in all melanoma cell lines (FIG. 8A). These results indicate that amongst the two categories of BH3-only proteins, the only “sensitizing” molecule was NOXA in melanoma cells following treatment with bortezomib. When the multiple-BH related family members were examined, none of these proteins were consistently induced in all four melanoma cells by bortezomib (FIG. 8B). Normal melanocytes responded very differently to bortezomib (FIGS. 8A and 8B), and they did not exhibit NOXA induction in response to bortezomib (FIG. 8A).

Expanding the in vitro studies to in vivo studies, subcutaneous tumors produced in Nude mice (as described above in Example 7) were tested to detect NOXA using whole cell protein extracts as described above. Tumors of C8161 melanoma cells injected with PBS did not contain detectable NOXA, but injection of bortezomib (2.5 mg/kg) did induce NOXA in these treated tumors (FIG. 8C). Together the in vitro and in vivo data support a role for NOXA in the apoptotic response of melanoma cells to bortezomib.

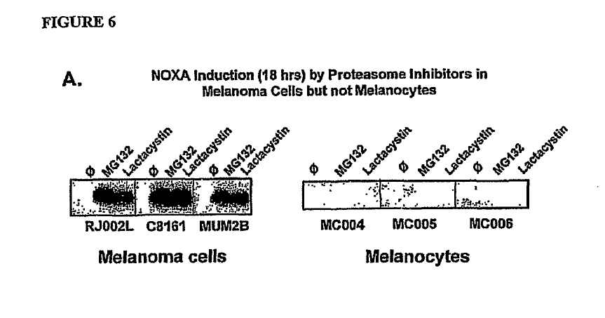

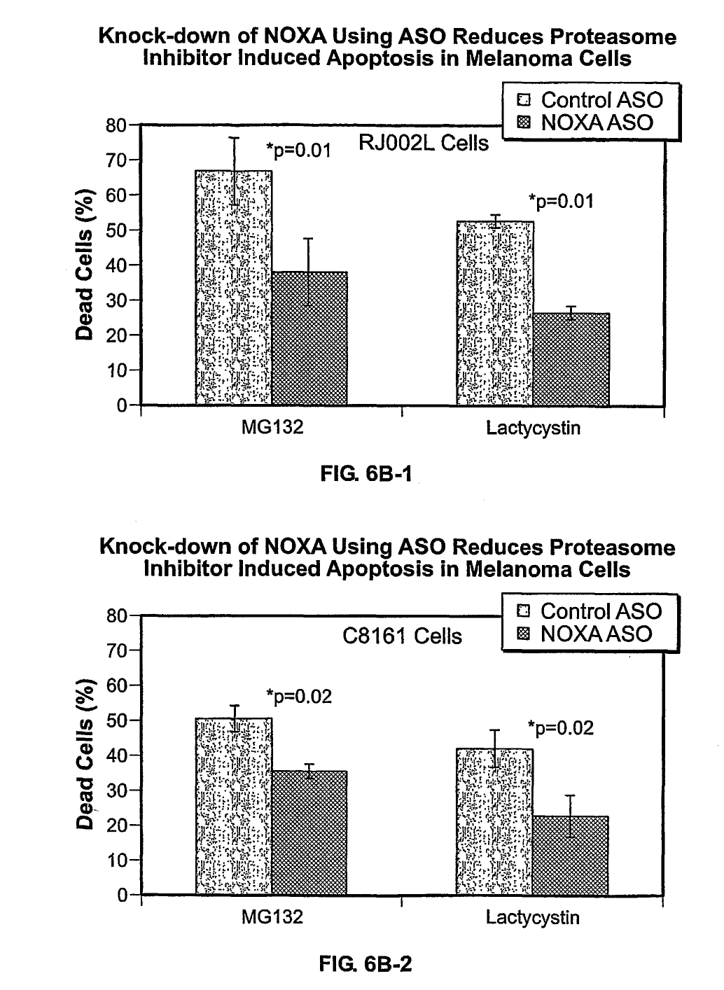

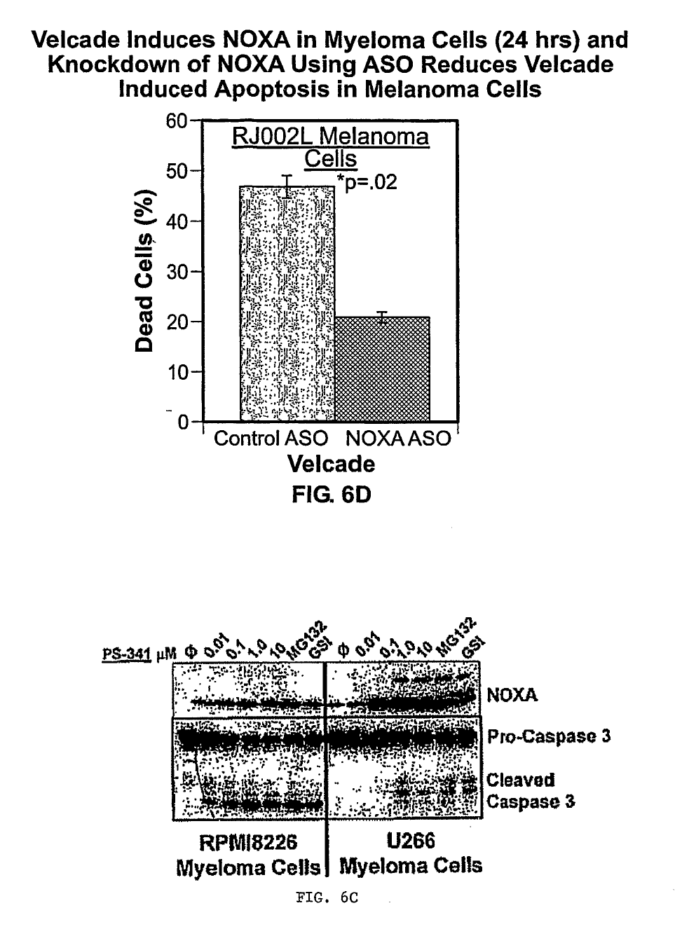

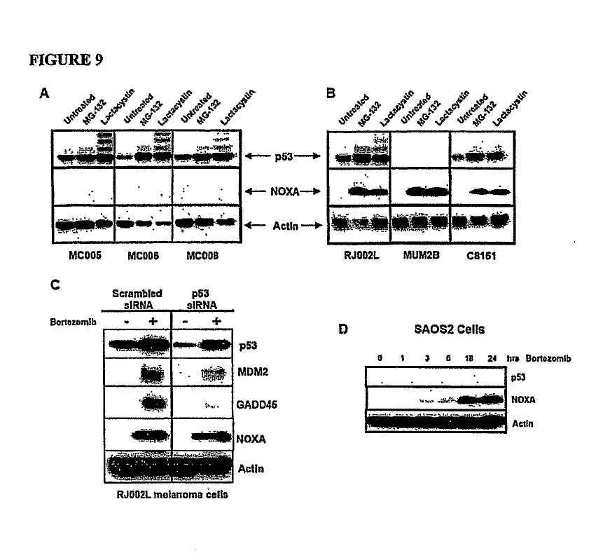

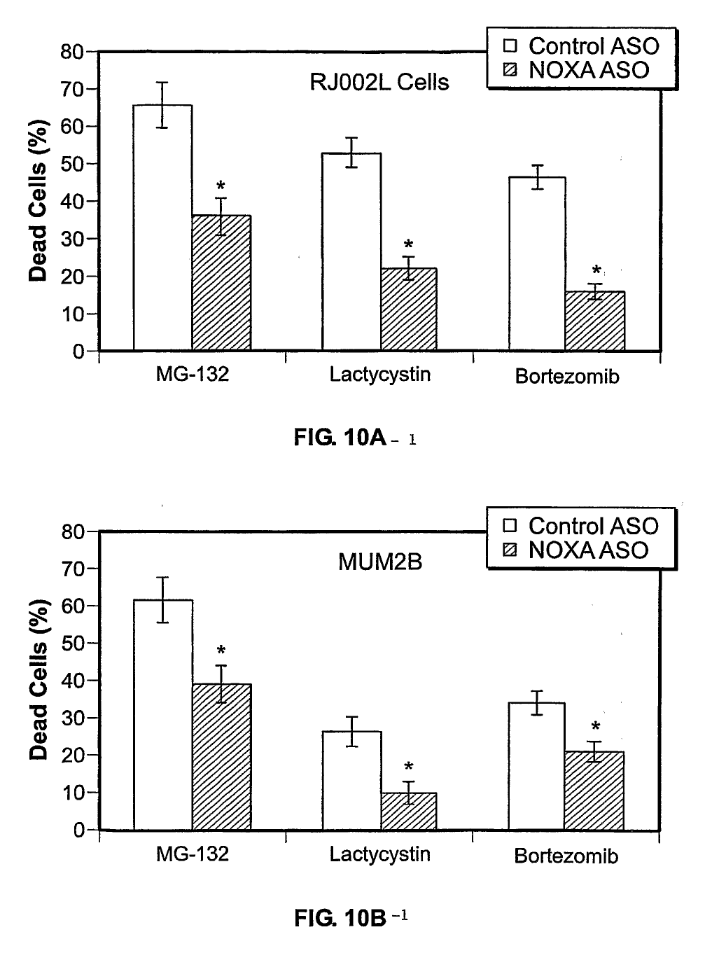

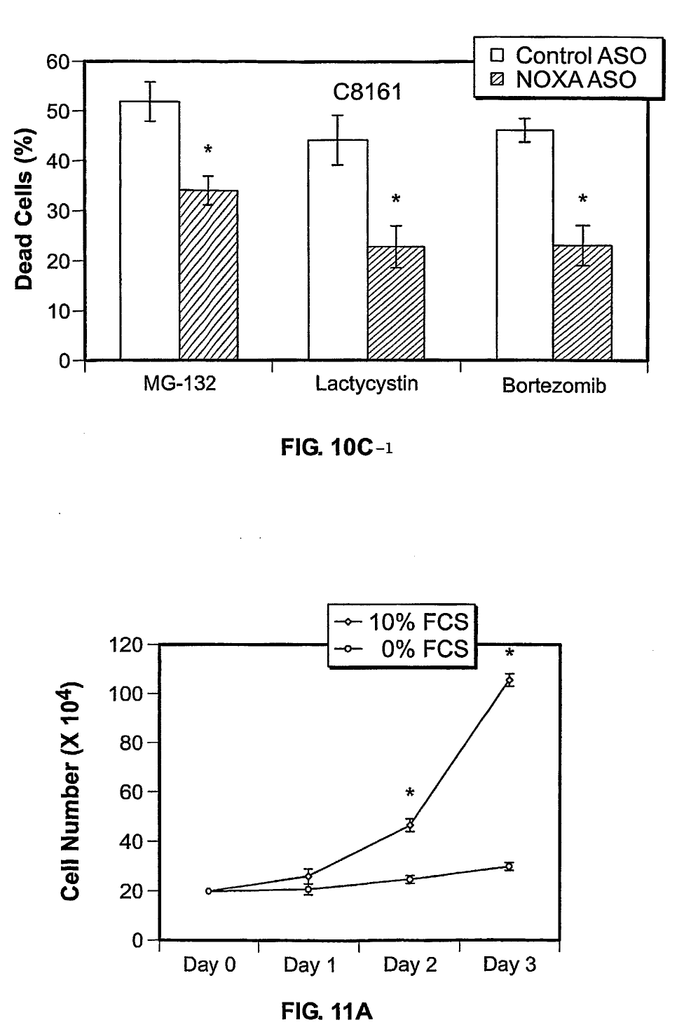

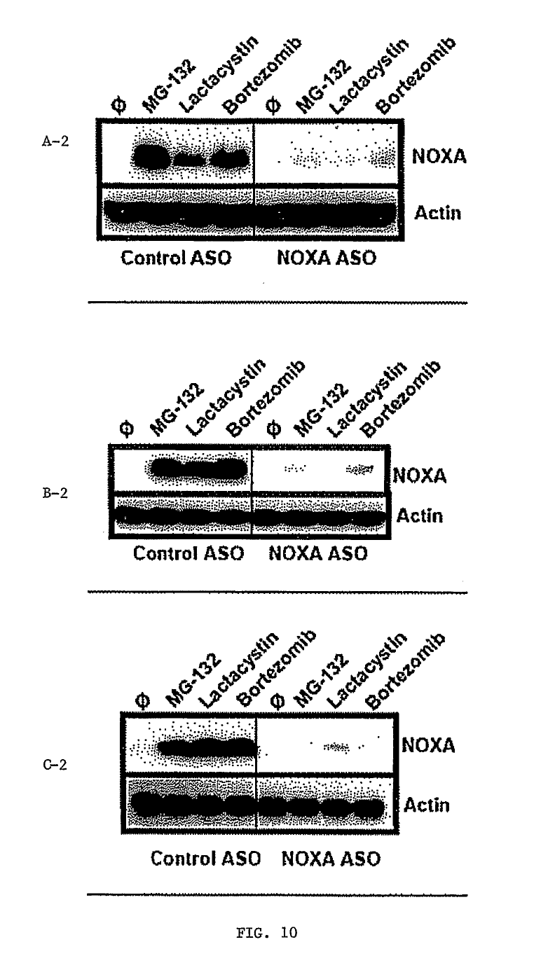

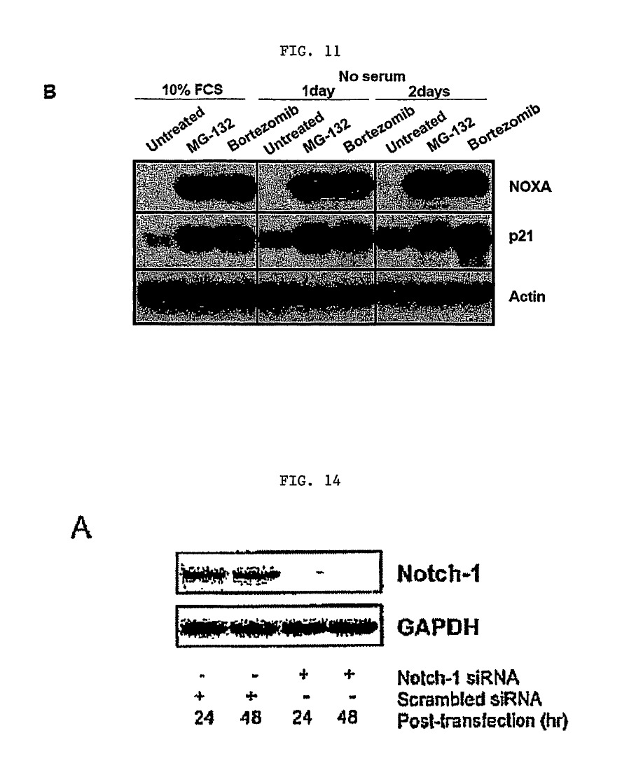

Two other proteasome inhibitors, lactasytsin and MG-132, were tested for the ability to induce NOXA in melanoma cells and melanocytes. RJ002L, C8161 and MUM2B melanoma cells, as well as four normal melanocyte cultures (MC-004, MC-005, MC-006 and MC-008) were examined. Prior to testing, the RJ002L melanoma cell lines were maintained in RPMI supplemented with 10% FBS. Late passage melanoma cell lines (C8161 and MUM2B) were utilized as previously described (Welch D et al., 1991, Int J Cancer 47:227-237; Bittner M et al., 2000, Nature 406:536-540). Normal human melanocytes were isolated from neonatal foreskins and cultured as previously described (Qin J et al., 2004, Mol Cancer Ther 3:895-902). Proliferating cells were examined by Western blot analysis before and after 18 hrs of exposure to MG-132 (1 μM; FIG. 9A or 10 μM; FIG. 6A) or lactacystin (1 μM; FIG. 9A or 10 μM; FIG. 6A). Also, since previous reports indicate that NOXA induction is p53-dependent (Bouillet P et al., 2001, Dev Cell 1:645-653; Oda E et al., 2000, Science 288:1053-1058; Shibue, T et al., 2003, Genes Dev 17:2233-2238, 2003), relative p53 levels were also examined. Antibodies were obtained as follows: p53 (SC-126) from Santa Cruz Biotechnology (Santa Cruz, Calif.); NOXA (OP180) from Calbiochem; and β-actin from ICN (Irvine, Calif.). Western blotting was performed as described above.

As shown in FIGS. 6A and 9A, there was no induction of NOXA in any of the normal melanocytes in the presence of 1 μM MG-132 or lactacystin (FIG. 9A) or 10 μM MG-132 or lactacystin (FIG. 6A), despite the accumulation of ubiquinated p53 consistent with inhibition of the proteasome activity (FIG. 9A). In contrast, all three of the melanoma cell lines treated with either 1 μM MG-132 or lactacystin (FIG. 9B) or 10 μM MG-132 or lactacystin (FIG. 6A) induced high NOXA levels.

To more definitively establish a role for NOXA in the proteasome inhibitor-induced apoptotic response, three different melanoma cell lines were pre-treated with an antisense oligonucleotides (ASO). The ASOs included a NOXA targeted sequence (5′-TCA GTC TAC TGA TTT ACT GG-3′) (SEQ ID NO: 2) and a universal scrambled control oligonucleotide (5′-TTC TAC CTC GCG CGA TTT AC-3′) (SEQ ID NO: 4) (FIGS. 10A, 10B and 10C), or control oligonucleotide (CO) (5′-CCT TCC CTG AAG GTT CCT CC-3′) (SEQ ID NO: 3) (FIGS. 6B and 6D). All three ASO were provided by ISIS Pharmaceuticals Inc. (Carlsbad, Calif.) as previously described by Qin et al., 2004, Mol Cancer Ther. 3:895-902). Melanoma cells were seeded at 2×10⁵ cells into 6 well plates 1 day before transfection. Opti-MEM (InVitrogen) was pre-incubated for 30 minutes at room temperature using a ratio of 3 μM/mL lipofectamine per 100 nmol/L to produce a final oligonucleotide concentration of 50 mmol/L. Cells were washed with PBS and transfection mix (1 ml) was added. After 4 hrs of incubation, RPMI 1640 (1 mL) containing 20% fetal bovine serum and the proteasome inhibitor (MG-132, lactacystin or bortezomib) was added at 1 μM (FIGS. 10A, 10B and 10C) or 10 μM (FIGS. 6B and 6D) and incubated for 24 hrs. The treated cells were analyzed for cell viability using APO TARGET Annexin V-FITC staining kits (Biosource) and by Western blot using NOXA and β-actin antibodies as described above.

While control ASO pretreated melanoma cells were sensitive to proteasome inhibitor-mediated apoptosis, the ability of the ASO-targeting NOXA to block NOXA induction was accompanied by significant reduction in the apoptotic response for all three proteasome inhibitors in all three melanoma cell lines (FIGS. 10A, 10B, 10C, 6B and 6D). The ASO-targeting NOXA was able to reduce the apoptotic response by approximately 30-50% depending on the proteasome inhibitor used and the type of melanoma cell line. Since the inhibition of apoptosis using ASO-targeting NOXA was incomplete, other components of the apoptotic machinery are likely to be involved. Nonetheless, these data indicate an important role for NOXA induction in mediating the killing of melanoma cells achieved by the use of proteasome inhibitors.

### Example 9

### Effect of Bortezomib on Osteosarcoma Cells

To test whether the NOXA inducing effect of bortezomib is limited to melanomas, SAOS2 cells (ATTC, Rockville, Md.), an osteogenic sarcoma cell line (p53-null) was exposed to 1 μM bortezomib. SAOS2 cells were tested for p53 expression and induction of NOXA expression before and during a 24 hr exposure to bortezomib (1 μM). The cells were assayed by Western blot using antibodies to NOXA, p53 and β-actin as described above. As shown in FIG. 9D, induction of NOXA occurred beginning at 3-6 hrs with more prominent levels detected at 18-24 hrs. These data indicate that bortezomib-induced NOXA expression is not limited to melanocytes, and that p53 is not absolutely required for NOXA induction.

### Example 10

### Relationship Between Proliferation and NOXA Induction In Vitro

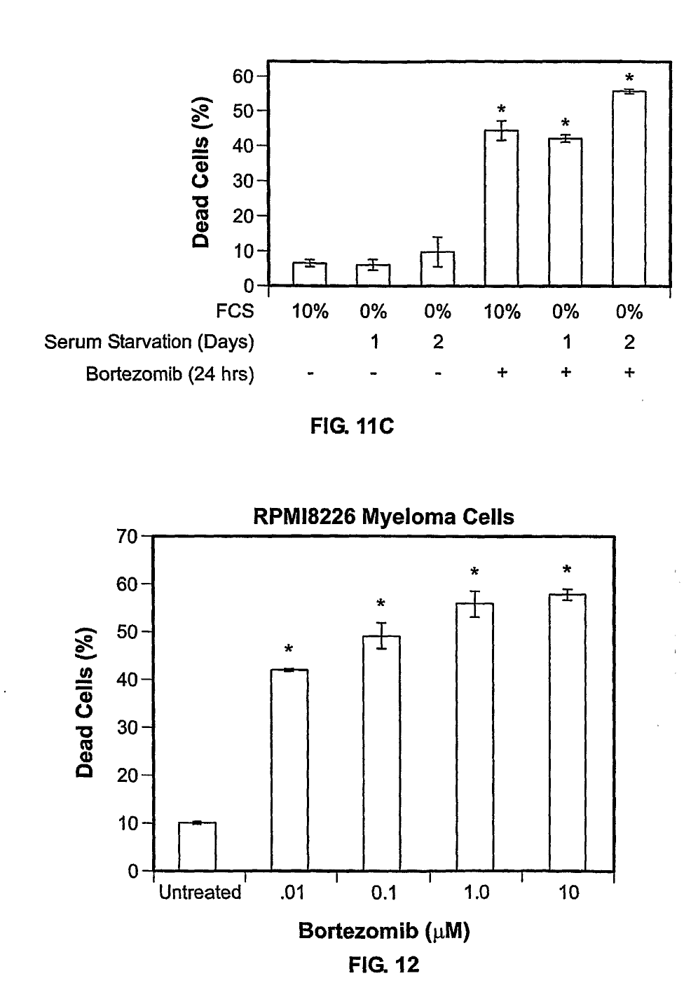

C8161 melanoma cells were induced into a relatively quiescent state by serum withdrawal. Proliferation assays were conducted in the presence or absence of 10% fetal calf serum (FCS) by manual counting of melanoma cells in triplicate wells on days 0, 1, 2 and 3. The proliferation assay revealed a minimal increase in cell number for C8161 melanoma cells after 2 and 3 days in serum free medium, compared to significantly increased cell number (p<0.05) in the presence of 10% FCS (FIG. 11A). By phase contrast microscopy, the withdrawal of FCS arrested the cells (data not shown). This growth arrest was confirmed by Western blots of the cells that were maintained as either rapidly proliferating cells in the presence of 10% FCS or were growth arrested by serum withdrawal and washing with PBS for either 1 or 2 days. Whole cell extracts were prepared and Western blot analysis was performed as described above in Example 8 using antibodies to NOXA, p21 (SC-817, Santa Cruz Biotechnology, Santa Cruz, Calif.) and β-actin as described here and above.

As shown in FIG. 11B, MG-132 and bortezomib were both able to trigger prominent NOXA induction in either proliferating melanoma cells (10% FCS) or in serum-deprived melanoma cells (no serum). Growth arrest induced by serum withdrawal was confirmed by induction of p21. Furthermore, the amount of cell death by apoptosis in bortezomib-treated, serum-deprived melanoma cells was comparable to that seen in bortezomib-treated proliferating melanoma cells, as measured using APO TARGET Annexin V-FITC staining kits (Biosource) as described above (FIG. 11C). Thus, not only can proteasome inhibitors selectively induce NOXA and kill melanoma cells and not kill melanocytes independent of p53, but melanoma cells are susceptible to killing even when maintained in a relatively quiescent state in vitro. The withdrawal of growth factors in the melanoma cells maintained in a serum-free environment indicates that proteasome inhibitors can induce NOXA and apoptosis in non-proliferating cells in an equivalent fashion as rapidly proliferating melanoma cells (FIG. 1B).

### Example 11

### Effect of Proteasome Inhibitors and a Gamma Secretase Inhibitor on Myeloma Cells In Vitro

Two different multiple myeloma cell lines (RMP18226 and U266) (ATCC, Rockville, Md.) were treated for 24 hours with various concentrations of the proteasome inhibitors bortezomib (0.01 to 10 μM) and MG-132 (10 μM), or the dual inhibitor z-Leu-Leu-Nle-CHO (10 μM). The cells were evaluated for induction of NOXA and an increase in cleaved caspase 3, by Western blot analysis and for cell viability using APO TARGET Annexin V-FITC staining kits (Biosource) as described above. The caspase 3 antibody was purchased from Abcam (Cambridge, Mass.). As shown in FIG. 6C, bortezomib induces NOXA in a concentration dependent manner and also activates caspase 3 by the cleaving of procaspase 3. Similarly, MG-132 and z-Leu-Leu-Nle-CHO were found to induce NOXA in the two different multiple myeloma cell lines (RPMI8226 and U266). At a concentration of 10 μM, bortezomib induces approximately equal NOXA levels compared to the other proteasome inhibitor tested (MG-132) and the dual inhibitor z-Leu-Leu-Nle-CHO. The induction of NOXA coincided with the appearance of cleaved caspase 3, which is involved in the final stage of cell death. The two proteasome inhibitors and the dual inhibitor z-Leu-Leu-Nle-CHO all triggered significant apoptotic response after 18 hrs in the myeloma cell line RPMI8226. FIG. 12 is graph showing representative results from bortezomib treatment. The induction of apoptosis is concentration-dependent (FIG. 12); with doses as low as 0.01 μM bortezomib trigger significant apoptosis (p<0.05).

### Example 12

### Notch-1 siRNA Potentiates Cisplatin-Induced Growth Inhibition and Apoptosis in Cervical Cancer Cells

The human papilloma virus (HPV)-positive human cervical cancer cell line CaSki was obtained from ATCC (Rockville, Md.) and cultured in DMEM medium with 10% FBS. Double-stranded synthetic 21-mer RNA oligonucleotides (siRNAs) were purchased from Dharmacon (Lafayette, Colo.). The most effective sequence for Notch-1,5′-AAG TGT CTG AGG CCA GCA AGA-3′ (SEQ ID NO: 1), was selected from pilot experiments. An siRNA having the sequence 5′-AAC AGT CGC GTT TGC GAC TGG-3′ (SEQ ID NO: 5), which does not match any know mammalian GENBANK sequences, was used as a “scrambled” control in all experiments. Sequence specificity was determined by BLAST searches for the uniqueness. Transfection of CaSki human cervical cancer cells with siRNAs (200 nM siRNA oligos/60 mm dish) was performed by using Oligofectamine Transfection Reagents (InVitrogen) according to manufacturer's instructions.

At 24 and 48 hours after transfection with Notch-1 siRNA or scrambled control siRNA, the CaSki cells were assayed for Notch-1 and Notch-4 expression by Western blotting. Immunoblotting was performed using antibodies to Notch-1 (C-20), and Notch-4 (H-225) from Santa Cruz Biotechnology (Santa Cruz, Calif.). Secondary anti-goat, anti-mouse, anti-rabbit and anti-rat antibodies (IgG-HR1) were purchased from Vector Laboratories (Burlingame, Calif.), Santa Cruz Biotechnology and Amersham Biosciences. For Western blot analysis, equal amounts of protein extracted from each sample (30 μg) were loaded into pre-cast 7% NuPAGE Tris-acetate gels or 4-12% NuPAGE Bis-tris gels (Invitrogen). Proteins were separated by SDS-PAGE and transferred to polyvinylidene difluoride membranes (Bio-Rad, Hercules, Calif.) using Invitrogen's buffering system. The membrane was incubated with indicated primary antibodies, washed, and visualized by incubation with horseradish peroxidase-conjugated secondary antibodies and chemiluminescent reagents (Roche Diagnostics Corporation, Indianapolis, Ind.).

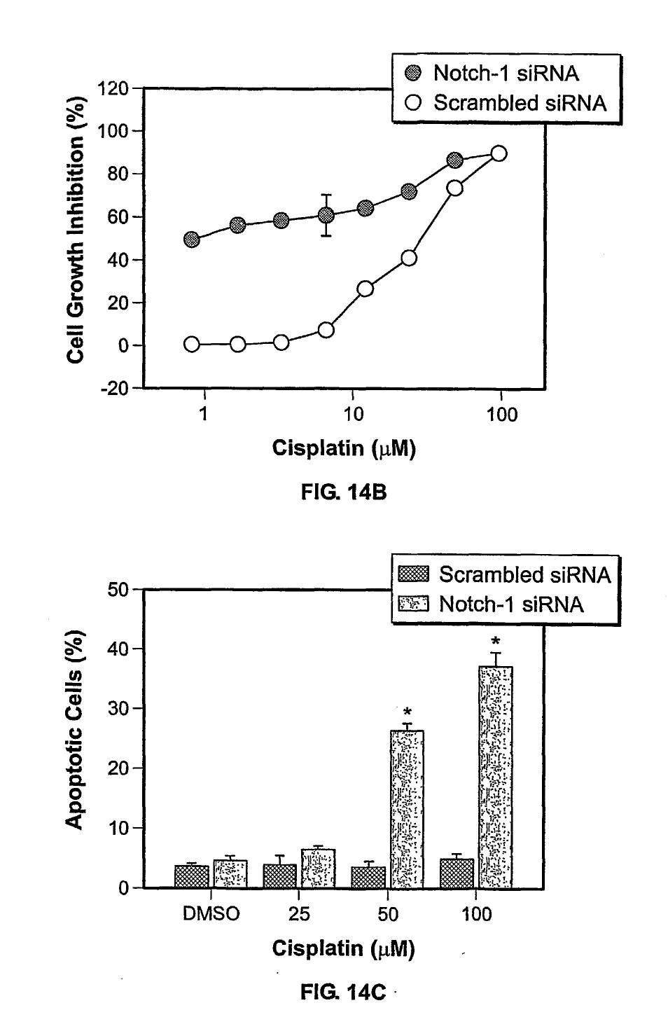

Introduction of Notch-1 siRNA into CaSki human cervical cancer cells was effective in genetically inhibiting Notch-1 signaling (FIG. 14A), but did not effect levels of Notch-4 (data not shown). This suggests that Notch-4 expression is regulated independently of Notch-1 in cervical cancer cells.

Twenty-four hours after transfection with Notch-1 siRNA or scrambled control siRNA, the CaSki cells were treated with various concentration of cisplatin before being collected for testing in a growth inhibition assay, and for measurement of the level of apoptosis by staining and assaying by flow cytometry.

The cell growth inhibition assay was performed in a 96-well plate format. Inhibition was assessed by monitoring the protein content in each well using the crystal violet staining method as described by Skehan et al., 1990, J Natl Cancer Inst 82:1107-1112 and Prochaska et al., 1988, Anal Biochem 169:328-336. Briefly, cells were plated at a density of 2×10⁴ cells/ml in 200 μl of DMEM supplemented with 10% FBS. The cells were transfected with siRNAs (2 mM siRNA oligos/well) using Oligofectamine Transfection Reagents (InVitrogen) according to manufacturer's instructions. Twenty-four hours post-transfection, the media was decanted, 190 μl of fresh media was added to each well, and 10 μl of cisplatin (4 mM) dissolved in 10% DMSO was added to the wells as a series 2-fold dilutions (final concentration of cisplatin in the wells ranged from 0.5-200 μM). The cells were incubated with cisplatin for 48 hrs, the media was decanted, and the cells were submerged with 200 μl of 0.2% crystal violet in 2% ethanol for 10 min. The plates were rinsed for 2 min with tap water. The bound dye was solublized by incubation at 37° C. for 1 h with 200 μl of 0.5% sodium dedecyl sulfate in 50% ethanol. The plates were then scanned with an ELISA reader at 562 nm. The percentage cell growth inhibition mediated by cisplatin was calculated as: % inhibition=[1-(OD of cisplatin-treated cells/OD DMSO-treated control cells)]×100/100.

For the apoptosis measurement assay, the transformed CaSki human cervical cancer cells were treated with DMSO, or 25 μM, 50 μM or 100 μM cisplatin for 24 hours. After cisplatin treatment, the cells were collected and stained with fluorescein-conjugated annexin V and propidium iodide, using the APO TARGET Annexin-V FITC Apoptosis Kit (BioSource International, Inc., Camarillo, Calif.) according to the manufacturer's instructions. After the staining, the cells were sorted by flow cytometry with a FACScan instrument (Becton-Dickinson), Annexin-V positive stained cells were counted as apoptotic cells. Cells that were propidium iodide positive but annexin V negative were not counted as apoptotic cells.

Notch-1 siRNA, when used in combination with cisplatin, a drug commonly used in the treatment of advanced cervical cancer, strikingly potentiated the cytotoxicity of cisplatin, as measured by the cell growth inhibition assay of Skehan P et al, 1990, lowering the IC₅₀ of cisplatin in CaSki cells by almost 2 orders of magnitude (from 27.3 μM to 0.46 μM) (FIG. 14B). Annexin V flow cytometry (FIG. 14C) showed that cytotoxicity was largely due to apoptotic cell death.

### Example 13

### z-Leu-Leu-Nle-CHO Potentiates Cisplatin-Induced Cell Growth Inhibition and Apoptosis in Cervical Cancer Cells

In light of the results showing biological inhibition of Notch signaling acting synergistically with cisplatin, CaSki human cervical cancer cells were tested to determine if pharmacological inhibition of Notch signaling also acted synergistically with cisplatin.

The CaSki human cervical cancer cells were incubated for 24 h with various concentrations (0-5 μM) of the dual inhibitor z-Leu-Leu-Nle-CHO (Calbiochem) (FIG. 13A). The cells were then collected and assayed for Notch-1 and Notch-4 expression by Western blotting. Immunoblotting was performed using antibodies to the transmembrane portion of Notch-1 (N^{TM}) (Santa Cruz Biotechnology (Santa Cruz, Calif.)), or the intracellular portion of Notch-1 (N^{IC}) (Abcam, Inc. (Cambridge, Mass.)). Secondary anti-goat, anti-rnouse, anti-rabbit and anti-rat antibodies (IgG-HRP) were purchased from Vector Laboratories (Burlingame, Calif.), Santa Cruz Biotechnology and Amersham Biosciences. For Western blot analysis, equal amounts of protein extracted from each sample (20 μg) were loaded into pre-cast 7% NuPAGE Tris-acetate gels or 4-12% NuPAGE Bis-tris gels (Invitrogen). Proteins were separated by SDS-PAGE and transferred to polyvinylidene difluoride membranes (Bio-Rad, Hercules, Calif.) using Invitrogen's buffering system. The membranes were incubated with the indicated primary antibodies, washed, and visualized by incubation with horseradish peroxidase-conjugated secondary antibodies and chemiluminescent reagents (Roche Diagnostics Corporation, Indianapolis, Ind.).

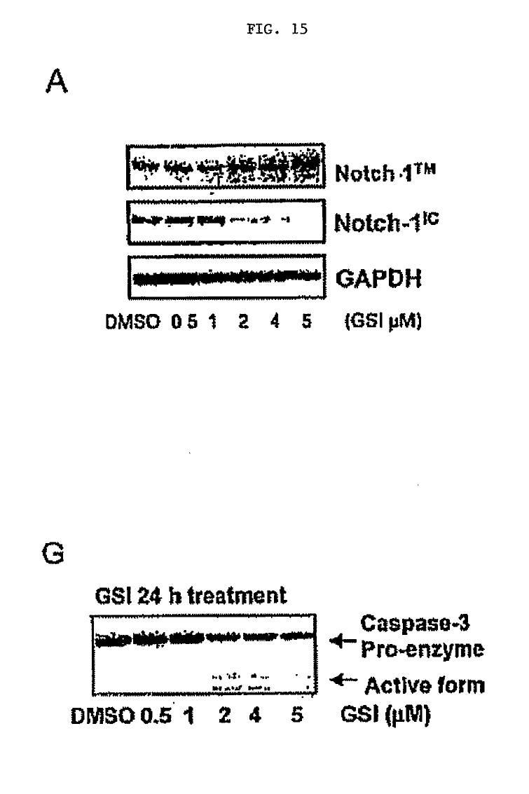

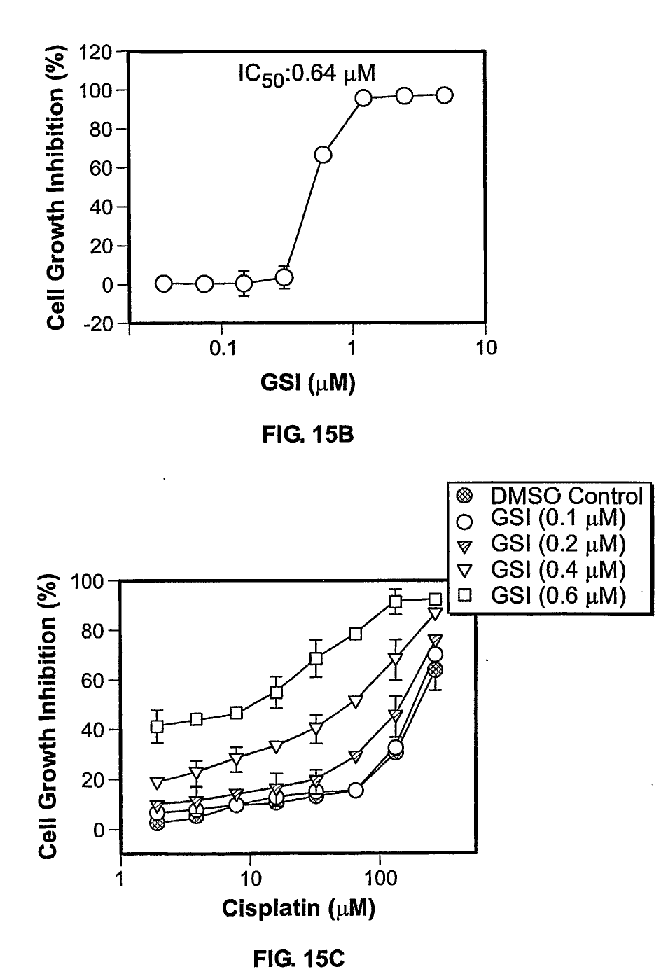

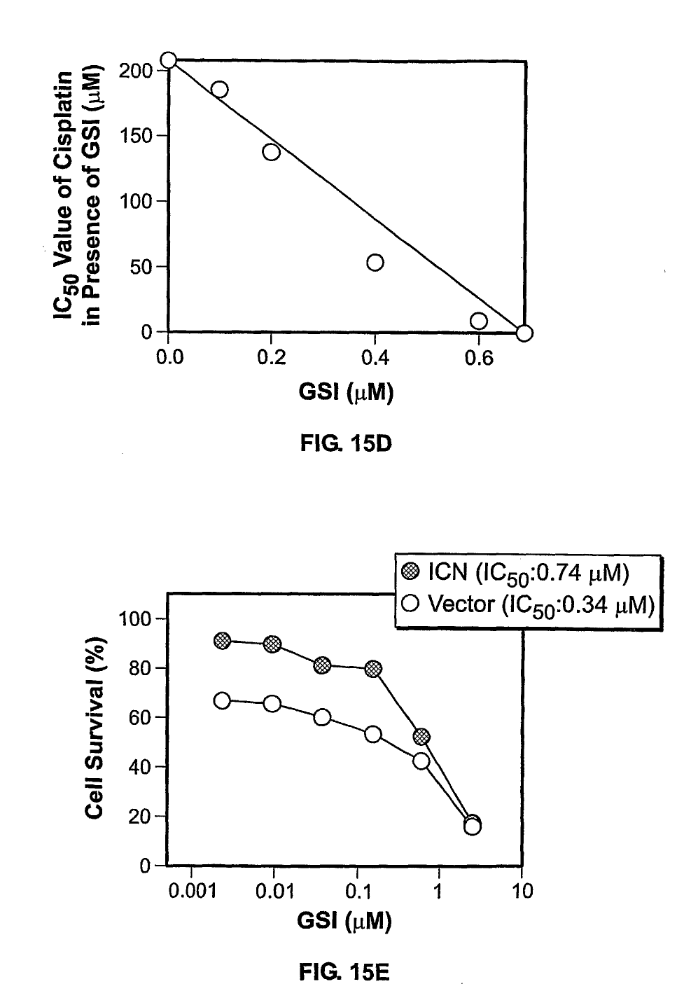

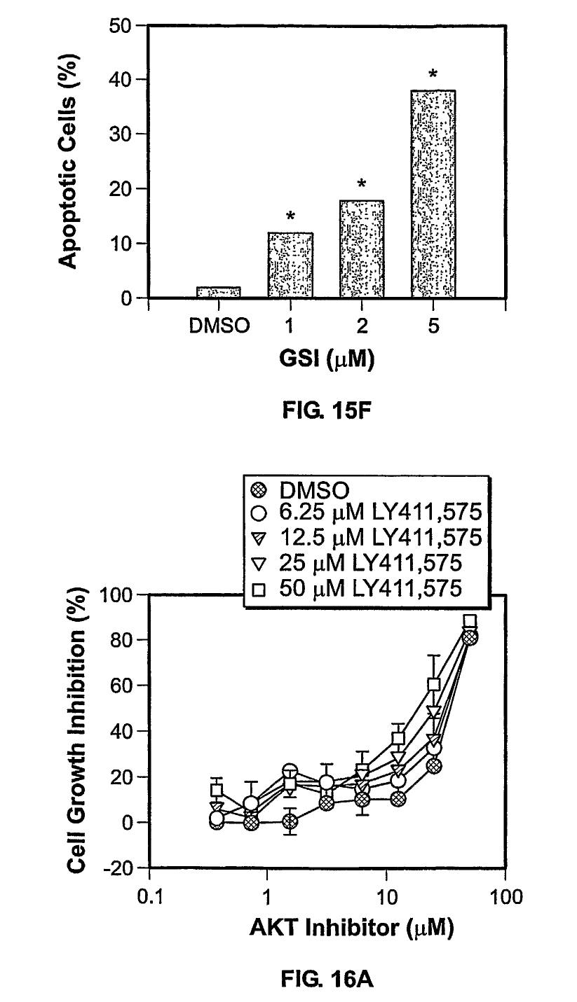

As seen in FIG. 15A, the dual inhibitor z-Leu-Leu-Nle-CHO caused a dose-dependent reduction in cleaved Notch-1, with a corresponding accumulation in transmembrane Notch-1.

CaSki human cervical cancer cells treated for 48 hrs with z-Leu-Leu-Nle-CHO alone were tested for cell growth inhibition. The cell growth inhibition assay was performed in a 96-well plate format. Inhibition was assessed by monitoring the protein content in each well using the crystal violet staining method Skehan, P. et al., 1990, J Natl Cancer Inst 82:1107-1112. Briefly, cells were plated at a density of 2×10⁴ cells/ml in 200 μl of DMEM supplemented with 10% FBS. After pre-incubation period of 24 h, the media was decanted, 190 μl of fresh media was added to each well, and 10 μl (150 μM) of z-Leu-Leu-Nle-CHO dissolved in 10% DMSO was added to the wells as a series 2-fold dilutions (final concentration of cisplatin in the wells ranged from 0.05-7.5 μM). The cells were incubated with z-Leu-Leu-Nle-CHO for 48 hrs, the media was decanted, and the cells were submerged with 200 μl of 0.2% crystal violet in 2% ethanol for 10 min. The plates were rinsed for 2 min with tap water. The bound dye was solublized by incubation at 37° C. for 1 h with 200 μl of 0.5% sodium dedecyl sulfate in 50% ethanol. The plates were then scanned with an ELISA reader at 562 nm. The percentage cell growth inhibition mediated by test agents was calculated as: % inhibition=[1-(OD of z-Leu-Leu-Nle-CHO-treated cells/OD DMSO-treated control cells)]×100/100. As shown in FIG. 15B, z-Leu-Leu-Nle-CHO alone is cytotoxic for CaSki cells, with an IC₅₀=0.64 μM.

To determine whether z-Leu-Leu-Nle-CHO treatment potentiates the effect of cisplatin on human breast cancer cells, the CaSki cells were treated for 48 h with 0.1-0.8 μM z-Leu-Leu-Nle-CHO and various concentrations of cisplatin (2-fold dilutions of a cisplatin solution to produce final concentrations of 1.6-200 μM cisplatin). Following the z-Leu-Leu-Nle-CHO and cisplatin treatment, the cells were tested for growth inhibition as described above.

At doses lower than its IC₅₀ (0.64 μM), z-Leu-Leu-Nle-CHO potentiated the effects of cisplatin in cell growth inhibition assays (FIG. 15C), which is consistent with the siRNA data (see Example 12). Isobologram analysis was performed by measuring the IC₅₀ values of cisplatin in the presence of various concentrations of z-Leu-Leu-Nle-CHO. These IC₅₀ values were plotted with SigmaPlot, and a straight line was traced between the IC₅₀ values of cells treated with either cisplatin or z-Leu-Leu-Nle-CHO alone. IC₅₀ values that fall below the line indicate synergistic effects. Isobologram analysis indicated moderate synergism between z-Leu-Leu-Nle-CHO and cisplatin, especially at low cisplatin concentrations (1-50 μM) (FIG. 15D).

To confirm that the effect of z-Leu-Leu-Nle-CHO on the CaSki cells was mediated through Notch-1, CaSki cells were transfected with either a constitutively active Notch-1 (ICN) or vector in a 96-well format as described above. The Notch constructs have been previously described (Weijzen et al., 2002, Nat Med 8:979-986). Briefly, the constructs were in created in pLZRS (Kinsella and Nolan, 1886, Hum Gene Ther. 17:1405-13.) including either the intracellular portion of Notch-1 or no insert (empty vector). The transfected cells were treated with various concentrations of z-Leu-Leu-Nle-CHO for 48 h and tested for growth inhibition using the APO TARGET Annexin-V FITC Apoptosis Kit (BioSource International, Inc., Camarillo, Calif.) as described above.

As shown in FIG. 15E, concomitant transfection of constitutively active Notch-1 significantly reversed the effects of z-Leu-Leu-Nle-CHO at concentrations below IC₅₀ in cytotoxicity assays. This indicates that the effects of z-Leu-Leu-Nle-CHO are at least in part mediated by Notch-1 inhibition.

To determine whether z-Leu-Leu-Nle-CHO induces cell death via apoptosis, CaSki cells were treated with various concentrations of z-Leu-Leu-Nle-CHO for 18 h and then either stained with Annexin-V-FITC as described above, or subject to Western blot analysis using antibody to both the pro-enzyme and active form of caspase-3 (Santa Cruz Biotechnology, Santa Cruz, Calif.) as described above.

Annexin V flow cytometry (FIG. 15F) and caspase 3 activation assays (FIG. 15G) indicate that cell death induced by z-Leu-Leu-Nle-CHO is primarily apoptotic and is accompanied by dose-dependent caspase 3 activation.

### Example 14

### A Benzodiazepine Gamma-Secretase Inhibitor Synergizes with Drugs that Inhibit AKT or the Proteasome in CaSki Cervical Cancer Cells

To evaluate the possibility that inhibition of Notch signaling may sensitize advanced cervical cancers to the effect of cisplatin chemotherapy because Notch-1 protects these cells from apoptosis through the AKT and NF-κB pathways, a non-peptide, benzodiazepine gamma-secretase inhibitor, LY411,575 (Wong et al., 2004, J Biol. Chem. 279:12876-82) was tested in combination with a AKT inhibitor (AKT inhibitor I) or a proteasome inhibitor (MG-132) for effects on cell growth. The latter class of drugs is thought to act primarily by preventing NF-KB activation (Adams, J, 2002, Curr Opin Oncol 14:628-634; Richardson, P, 2003, Cancer Treat Rev 29 Suppl 1:33-39) caused by proteasome-mediated degradation of IκB. LY411,575 was used in these experiments because it is less potent than z-Leu-Leu-Nle-CHO in vitro, and because its structure does not suggest that it could inhibit proteases other than γ-secretase.

To determine whether z-Leu-Leu-Nle-CHO treatment potentiates the effect of cisplatin on human breast cancer cells, the CaSki cells were treated for 48 h with 0.1-0.8 μM z-Leu-Leu-Nle-CHO and various concentrations of cisplatin (2-fold dilutions of a cisplatin solution to produce final concentrations of 1.6-200 μM cisplatin). Following the z-Leu-Leu-Nle-CHO and cisplatin treatment, the cells were tested for growth inhibition as described above.

To determine whether LY411,575 potentiates the effect of AKT inhibitor I or MG-132, CaSki cells were treated with (a) LY411,575 (6.25 μM to 50 μM) and AKT inhibitor 1 (0.5 μM to 75 μM), or (b) LY411,575 (1.5 μM to 200 μM) and MG-132 (0.05 μM to 0.2). Following the drug treatment, the cells were tested for growth inhibition as described above. LY411,575 was synthesized according to methods well known in the art (e.g., see WO-09828268, which is incorporated by reference), and AKT inhibitor I and MG-132 were purchased from Calbiochem.

Isobologram analysis was also performed to determine whether the drug combinations had synergistic effects on cell growth inhibition. The IC₅₀ values of LY411,575 in the presence of various concentrations of AKT inhibitor I or MG-132 were measured. These IC₅₀ values were plotted with SigmaPlot, and a straight line was traced between the IC₅₀ values of cells treated with either drug alone. IC₅₀ values that fall below the line indicate synergistic effects.

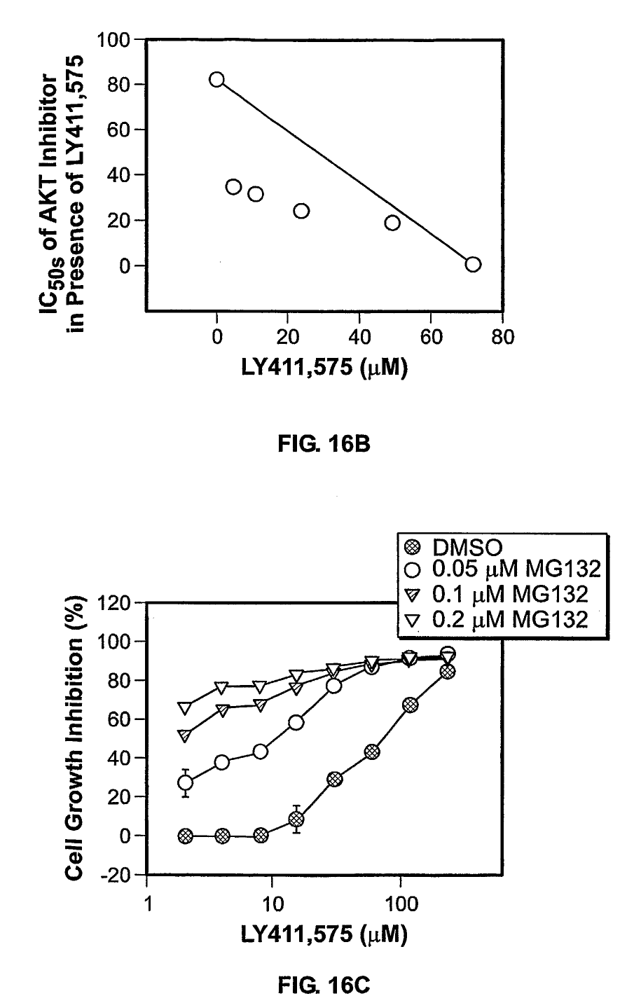

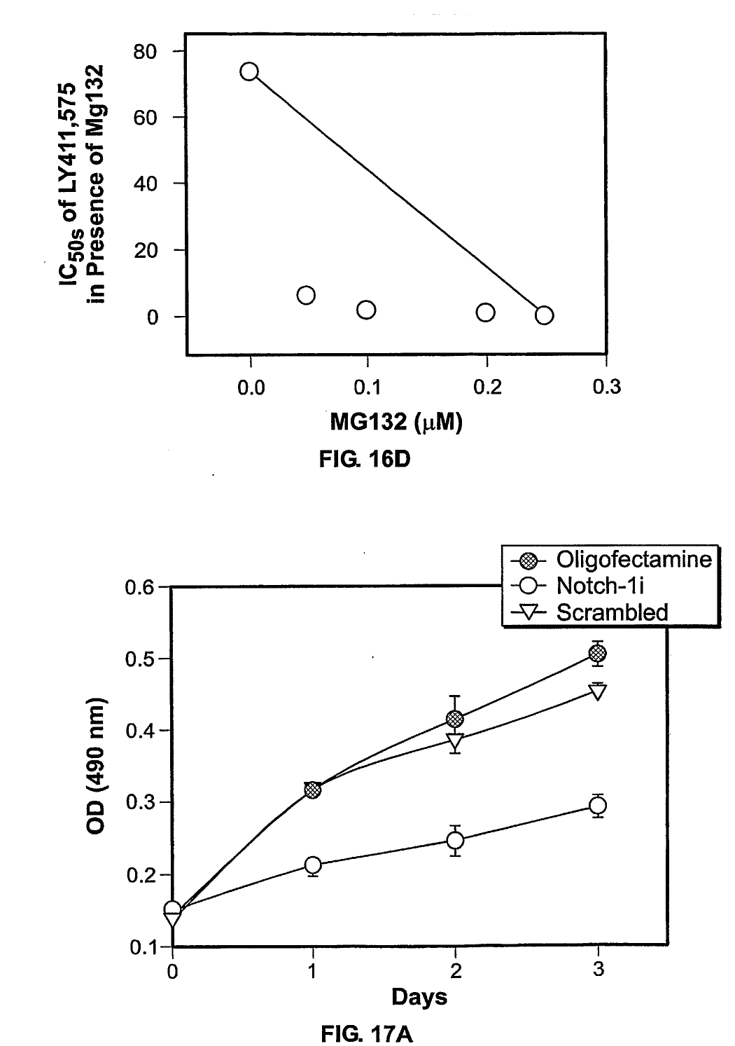

AKT inhibitor I has synergistic effects with the gamma-secretase inhibitor LY411,575 in cytotoxicity assays (FIGS. 16A and 16B). Similarly, the combination of MG-132 and LY411,575 showed striking synergism in CaSki cells (FIGS. 16C and 16D). Taken together, these data suggest that a combined treatment with Notch inhibitors and either AKT or proteasome inhibitor may have therapeutic uses in the treatment of advanced cervical cancer.

### Example 15

### Genetic Inhibition of Notch Signaling Inhibits Proliferation and Extracellular Matrix Invasion in Breast Cancer Cells

An invasive, estrogen-independent breast cancer cell line that spontaneously expresses high levels of Notch-1 and -4, MDA-MB231, and normal human breast cells were used to investigate whether inhibition of Notch activation or expression affects the biological behavior of breast cancer cells. Human Mammary Epithelial Cells (HMEC) were purchased from Clonetics and cultured in mammary epithelial cell basal medium (MEBM) supplemented with 52 μg/ml Bovine Pituitary Extract (APE), 10 μg/ml human recombinant Epidermal Growth Factor (hEGF), 5 μg/ml insulin, 0.5 μg/ml Hydrocortisone, 50 μg/ml Gentamicin and 50 μg/ml Amphotericin-B. MDA-MB231 (ATCC, Rockville, Md.) were cultured in DMEM medium with 10% FBS. Notch expression in HMEC and MDA-MB231 cells was silenced using highly specific RNAi. Double-stranded synthetic 21-mer RNA oligonucleotides (siRNAs) were purchased from Dharmacon (Lafayette, Colo.). The most effective sequences were selected in pilot experiments and were as follows: Notch-1,5′-AAG TGT CTG AGG CCA GCA AGA-3′ (SEQ ID NO: 1); and Notch-4,5′-AAC CCT GTG CCA ATG GAG GCA-3′ (SEQ ID NO: 6). A control siRNA that does not match any known mammalian GENBANK sequences (Dharmacon) was used in all experiments: 5′-AAC AGT CGC GTT TGC GAC TGG-3′ (SEQ ID NO: 5). Sequence specificity was determined by BLAST searches which determined the uniqueness of the sequences. Transfection of siRNAs was performed using Oligofectamine (Invitrogen) as recommended by the siRNA manufacturer (Dharmacon). Transfection efficiency was optimized and followed on parallel wells using DNA double stranded oligos of identical sequence to the siRNAs, labeled with biotin on one strand. Transfected cells were identified by Streptavidin-horseradish peroxidase staining (Vectastain) and counted. Transfection efficiencies around 80% were routinely obtained.

Cytotoxicity and growth of transfected cells were estimated by a standard assay used for cancer drug screening, through the total amount of TCA-precipitated cell proteins after drug treatment (Likhitwitayawuid, K et al., 1993, J Nat Prod 56:30-38): 200 μl of cell suspension (10⁵ cells/ml) were added to each well in a 96-well plate. After incubation at, 37° C., in 5% CO₂ (30 minutes to 72 hours depending on the time course chosen), 50 μl 50% trichloroacetic acid was added to each well. After incubation at 4° C. for 1 hour, precipitates were washed with water 4-5 times and air dried overnight. One hundred μl of SRB solution (0.4% sulforhodamine B in 1% acetic acid, filtered before use) was added to each well for 30 minutes. Wells were washed with 1% acetic acid 4-5 times and air dried. Two hundred μl 10 mM Tris base was added to each well. Plates were shaken for 5-10 minutes and OD at 490 nm (OD₄₉₀) was recorded via ELISA reader. To assay matrix invasion, 300 μl of cell suspension (10⁶ cells/mL in serum-free medium) were added to invasion inserts (BioCoat Matrigel Invasion Chambers, Becton Dickinson, Bedford, Mass.) and incubated for 22 hours at 37° C., 5% CO₂. Cells were stained with 0.9% crystal violet in 10% ethanol for 20 minutes. Inserts were dipped in water several times and air dried. After removal of the non-invading cells from the interior of the inserts, stained invading cells were lysed in 10% acetic acid and OD₅₉₅ was determined.

Notch silencing was confirmed by Western blot using commercial goat polyclonal antibodies from Santa Cruz Biotechnology to Notch-1 (C-20, Cat# sc-6014 S) and Notch-4 (C-19, Cat# sc-8644). Total cell lysates were prepared as follows: cells grown on 6 cm dishes were scraped in lysis buffer containing 1×PBS, 1% Nonidet P40, 0.5% deoxycholate, 0.1% SDS, freshly added aprotinin (45 μg/ml), phenylmethylsulfonyl fluoride (10 μg/ml), and sodium orthovanadate (10 μM). Lysates were sonicated 4 times for 5 seconds each, followed by 30 minutes incubation on ice. Lysates were centrifuged at 10,000 g for 20 minutes at 4° C. Supernatants were used as total cell lysates. Nuclear extracts were prepared as follows: 10,000,000 cells were pelleted, washed, and quickly frozen in a dry ice bath. Pellets were thawed by adding 100 μl of buffer 1 (10 mM HEPES, pH 7.9, 10 mM KCl, 1.5 mM MgCl₂, 1 mM DTT). Nuclei were pelleted and lysed in 15 μl Buffer 2 (20 mM HEPES, pH7.9, 0.4 M NaCl, 1.5 mM MgCl₂, 25% glycerol, 0.2 mM EDTA, 1 mM DTT, and 0.5 mM PMSF). Lysates were diluted in 50-70 μl Buffer 3 (20 mM HEPES, pH 7.9, 50 mM KCl, 20% glycerol, 0.2 mM EDTA, 1 mM DTT, 0.5 mM PMSF). Protein concentrations were estimated using the BCA protein assay (Pierce). Supernatants were boiled in reducing SDS sample buffer (Novex). Twenty μg protein per lane were run on a 3-8% Tris acetate gel (Invitrogen) in Tris Acetate Reducing Running Buffer (Novex). Protein bands were transferred onto PVDF membranes (BioRad) using transfer buffer (Novex). Membranes were then blocked overnight at 4° C. in 2% blocking solution in TBS (Roche). Primary antibodies were diluted in 2% Boehringer blocking solution (total volume 5 ml). Membranes were incubated for one hour at room temperature with shaking, then washed 6 times for 10 minutes each in TBS wash buffer. Membranes were incubated in secondary antibody in 2% of blocking solution for 30 minutes, then washed 6 times for 10 minutes each at RT in wash buffer. Bands were detected with chemiluminescent reagent (Roche) according to the manufacturer's directions. Membrane were stripped in 62.5 mM Tris-HCl (pH 6.7) containing 2% SDS and 0.7% β-mercaptoethanol for 30 minutes at 55° C., blocked, and then reprobed as described above.

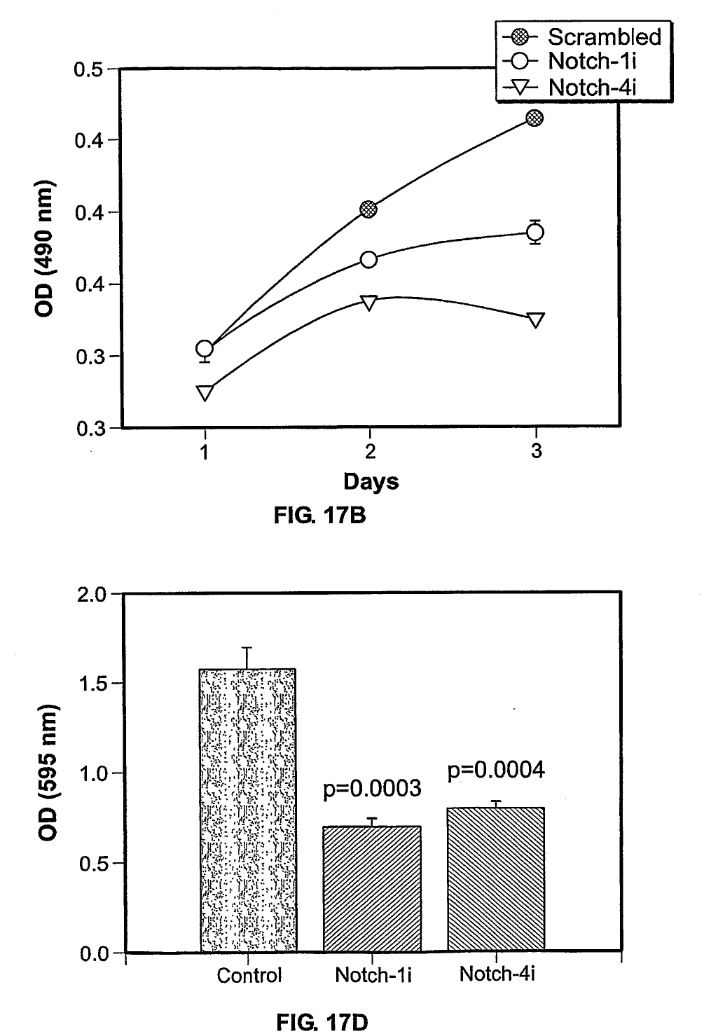

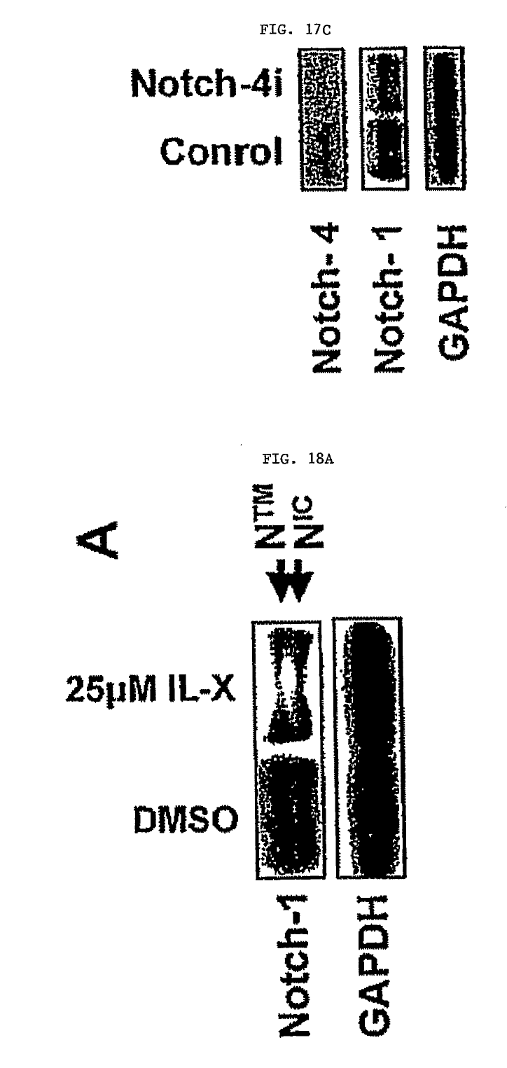

The Notch-1 targeted siRNA strongly inhibited the expression of both Notch-1 and -4 (data not shown). Notch-1 silencing (Notch-1i) inhibited proliferation without affecting survival in “normal” HMEC (FIG. 17A) and had a similar effect in MDA-MB231 cells (FIG. 17B). Notch-4 silencing (Notch-41) (FIG. 17C) caused an even stronger anti-proliferative effect in MDA-MB231 cells (FIG. 17B). Additionally, Notch-1 or Notch-4 silencing significantly inhibited extracellular matrix invasion, with similar potencies (FIG. 17D).

### Example 16

### Pharmacological Inhibition of Notch Signaling Inhibits Proliferation and Extracellular Matrix Invasion in Breast Cancer Cells

Because pharmacological agents may represent a more practical way of inhibiting Notch signaling in vivo for therapeutic purposes, two different gamma-secretase inhibitors were tested to determine whether these inhibitors can produce effects similar to those obtained by silencing Notch genetically (see Example 15). Tested gamma-secretase inhibitors included IL-X (cbz-IL-CHO) (Weijzen S et al., 2002, Nat Med 8:979-986, McLendon, C et al, 2000, FASEB J 14:2383-2386) which has previously been shown to have Notch-1-dependent anti-neoplastic activity in Ras-transformed fibroblasts, and a structurally related compound, z-Leu-Leu-Nle-CHO (FIG. 13A). IL-X was a kind gift from T. Golde (Mayo Clinic, Jacksonville, Fla.) and can be synthesized using techniques well know in the art (see e.g., McLendon et al, 2000, FASEB J 14:2383-2386). IL-X was dissolved in DMSO, aliquoted and stored at −80° C. Aliquots were thawed before use and not re-used. Gamma-secretase inhibitor z-Leu-Leu-Nle-CHO (Calbiochem 565750) was dissolved in DMSO, aliquoted and stored at −80° C.

Notch silencing was confirmed by Western blot as described above in Example 15 using commercial goat polyclonal antibodies from Santa Cruz Biotechnology to Notch-1 (C-20, Cat# sc-6014 S). For the Western blot analyses, MDA-MB231 cells were treated with 25 μM IL-X for 48 h or with 0.5, 1 or 2 μM z-Leu-Leu-Nle-CHO for 24 h. Cytotoxicity and matrix invasion assays were performed as described above in Example 15. For the cytotoxicity assays, MDA-MB231 cells were treated with 0-2000M IL-X for up to 3 days or with 0-20 μM z-Leu-Leu-Nle-CHO for up to 4 days. For the matrix invasion assays, MDA-MB231 cells were treated with 0, 25 or 50 μM IL-X for 22 h.

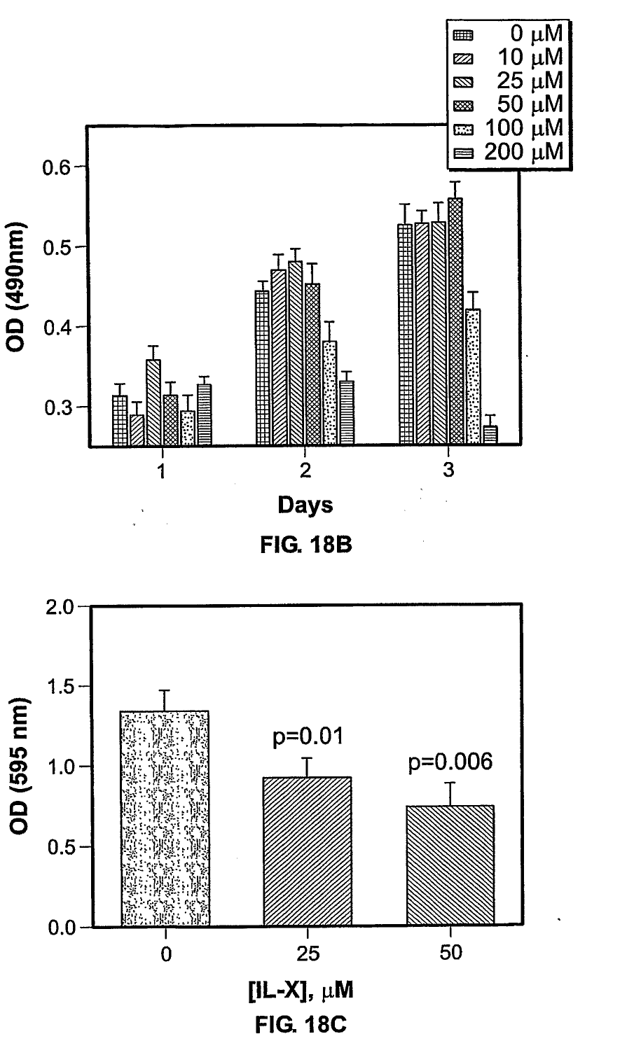

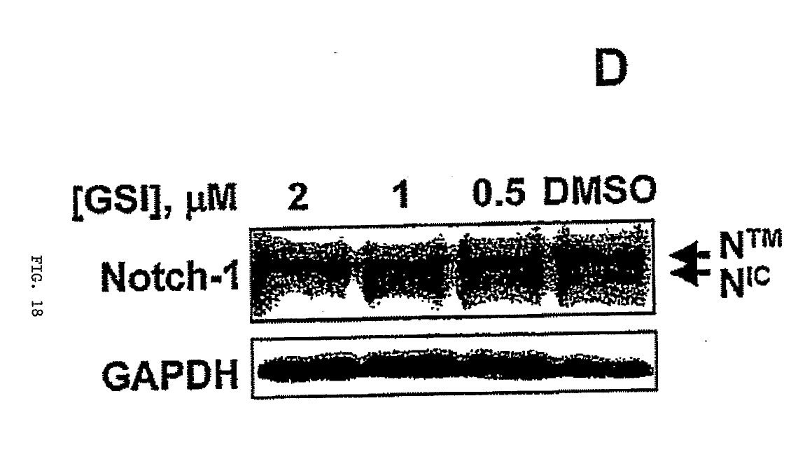

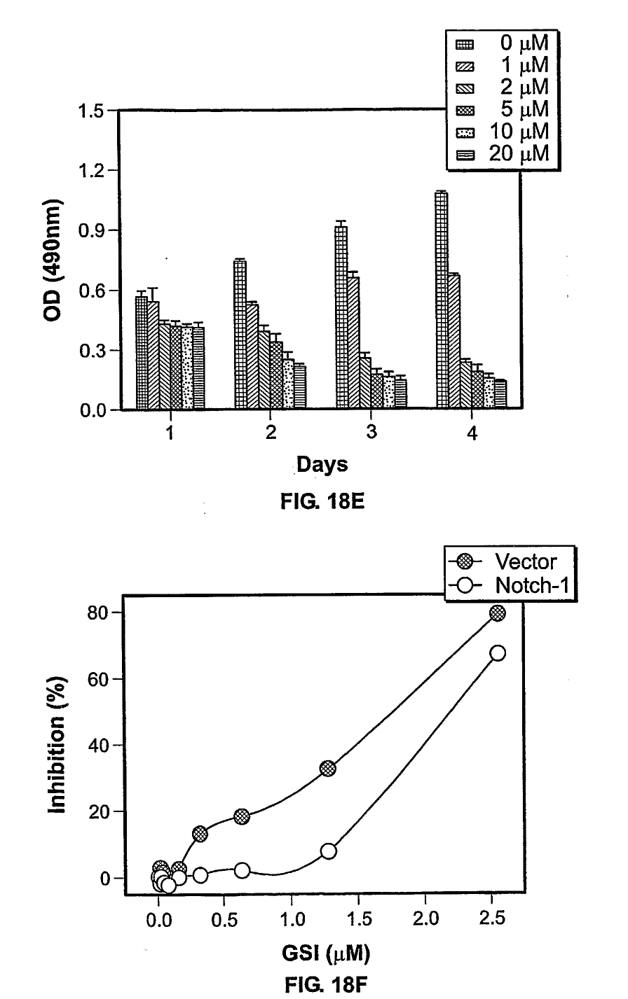

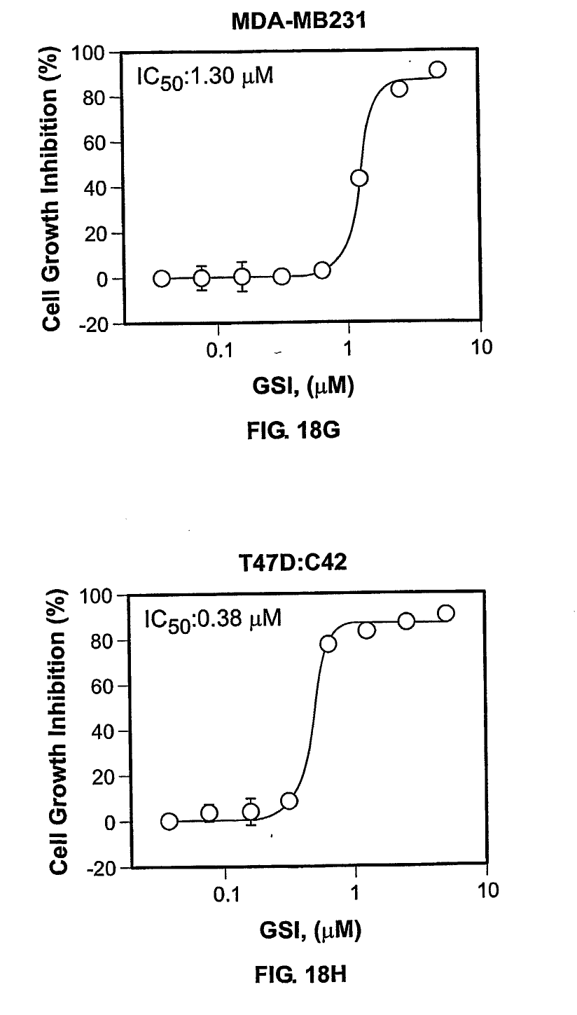

In MDA-MB231 cells, 25 μM IL-X caused an apparent reduction in all molecular forms of Notch-1 that was clearly evident after 48 hours of treatment (FIG. 18A). IL-X was cytotoxic for MDA-MB231 cells only above 100 μM (FIG. 18B). However, at 25 μM it significantly reduced in vitro matrix invasion of MDA-MB231 cells (FIG. 18C).

z-Leu-Leu-Nle-CHO caused a clear, dose-dependent decrease in N^{IC} and relative accumulation of N^{TM}, which was already evident at 24 h, and virtually complete at 2 μM (FIG. 18D). Consistent with this observation, z-Leu-Leu-Nle-CHO caused dose- and time-dependent cytotoxicity in MDA-MB231 cells that was statistically significant above 1 μM (FIG. 18E).

To test whether killing of the MDA-MB231 cells was due to Notch-1 inhibition, the cells were transiently transfected with either constitutively active Notch-1 (intracellular Notch-1 (Nlc)) or vector. The Notch constructs have been previously described (Weijzen S et al., 2002, Nat Med 8:979-986). Briefly, the constructs were in created in pLZRS (Kinsella and Nolan, 1996, Hum Gene Theo. 1:1405-13.) including either the intracellular portion of Notch-1 or no insert (empty vector). The constructs were used as plasmids and introduced into the MDA-MB231 cells by transfection as described above. Cytotoxicity assays were performed as described above in Example 15. Isobolograms were constructed using TableCurve (SPSS). For the cytotoxicity assays, MDA-MB231 cells were treated with 0-2.5 μM z-Leu-Leu-Nle-CHO for 48 h. As seen in FIG. 18F, the effects of z-Leu-Leu-Nle-CHO on MDA-MB231 cells were significantly rescued by concomitant transient transfection of constitutively active Notch-1. Since transfection efficiency in these experiments is never 100%, and 100% of the cells are exposed to z-Leu-Leu-Nle-CHO, the extent of rescue, though remarkable, is underestimated by these experiments.

Together with RNAi data (see Example 15), these findings suggest that genetic or pharmacological inhibition of Notch signaling have similar effects on MDA-MB231 cells, that Notch-4 mediates some of the biological effects of Notch-1 in these cells, and that Notch inhibition is a major mechanism of action of z-Leu-Leu-Nle-CHO in these cells.

### Example 17

### Inhibition of Proliferation in Breast Cancer Cells by z-Leu-Leu-Nle-CHO Inhibition of Notch Signaling Occurs Irrespective of p53 and ERα Status

To test whether p53 or ERα status was important for the effects of z-Leu-Leu-Nle-CHO in breast cancer cells, different breast cancer cells types were tested for proliferation in the presence of z-Leu-Leu-Nle-CHO. MDA-MB231 (p53-mutant, ERα-negative) cells were obtained from ATCC (Rockville, Md.). T47D:C42 (p53 wild-type, ERα-negative) and T47D:A18 (p53 wild-type, ERα-positive) cells were a kind gift of Debra Tonetti (University of Illinois-Chicago) and are described in Pin et al., 1996, Br. J. Cancer 74:1227-1236. T47D:C42 and T47D:A18 cells were propagated in RPMI 1640 with 10% FBS, 100 μM non-essential amino acid and 6 ng/ml insulin. Cytotoxicity assays were performed as described above in Example 15. Isobolograms were constructed using TableCurve (SPSS). For the cytotoxicity assays, the three types of breast cancer cells were treated with 0.05-10 μM z-Leu-Leu-Nle-CHO for 48 h.

FIGS. 18G-18I show that z-Leu-Leu-Nle-CHO treatment for 48 h caused dose-dependent growth arrest not only in p53-mutant, ERα-negative MDA-MB231 cells, but also in T47D:C42 cells (p53 wild-type, ERα-negative) as well as T47D:A18 cells (p53 wild-type, ERα-positive). Thus, γ-secretase inhibition was effective irrespective of p53 and ERαstatus.

### Example 18

### Pharmacological or Genetic Inhibition of Notch Signaling Causes Growth Arrest in G2/M in Breast Cancer Cells

Cell cycle analysis was performed to further study the effects of inhibition of Notch signaling in breast cancer cells. MDA-MB231 cells were treated overnight with 0-10 μM z-Leu-Leu-Nle-CHO prior to cell cycle distribution analysis by flow cytometry. Alternatively, MDA-MB231 cells were transfected with Notch-1, Notch-4 or control siRNA as described above in Example 15. For cell cycle distribution analysis by flow cytometry, z-Leu-Leu-Nle-CHO- or siRNA-treated cells (10⁶) were pelleted and washed twice in PBS. Cells were then fixed in 80% methanol and stored at −20° C. until use. Fixed cells were pelleted and washed twice in PBS, and resuspended in 50 μl PI:PBS solution (50 μg/ml PI). After RNase A (100 μg/ml) treatment at 37° C. for an hour, cells were analyzed by flow cytometry using a FACScalibur instrument.

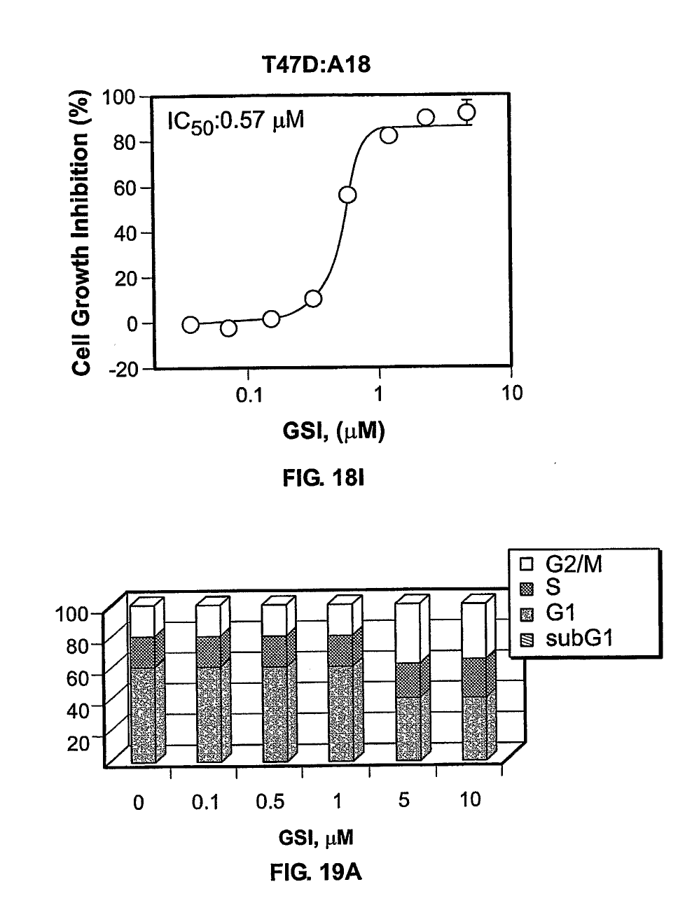

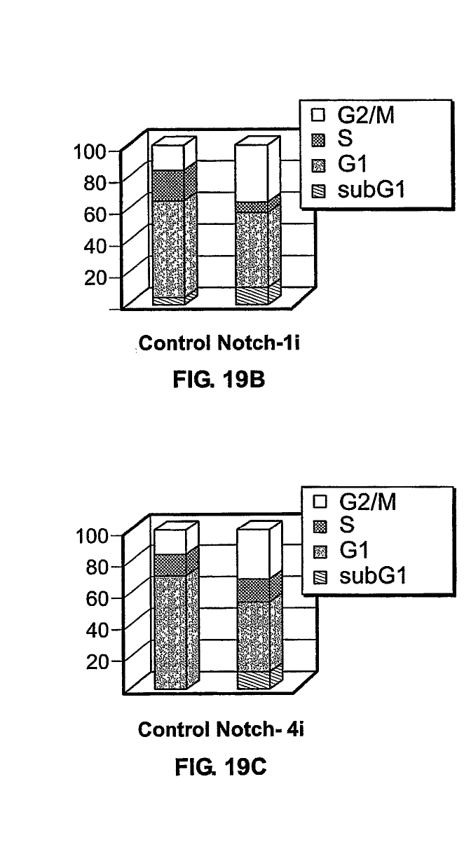

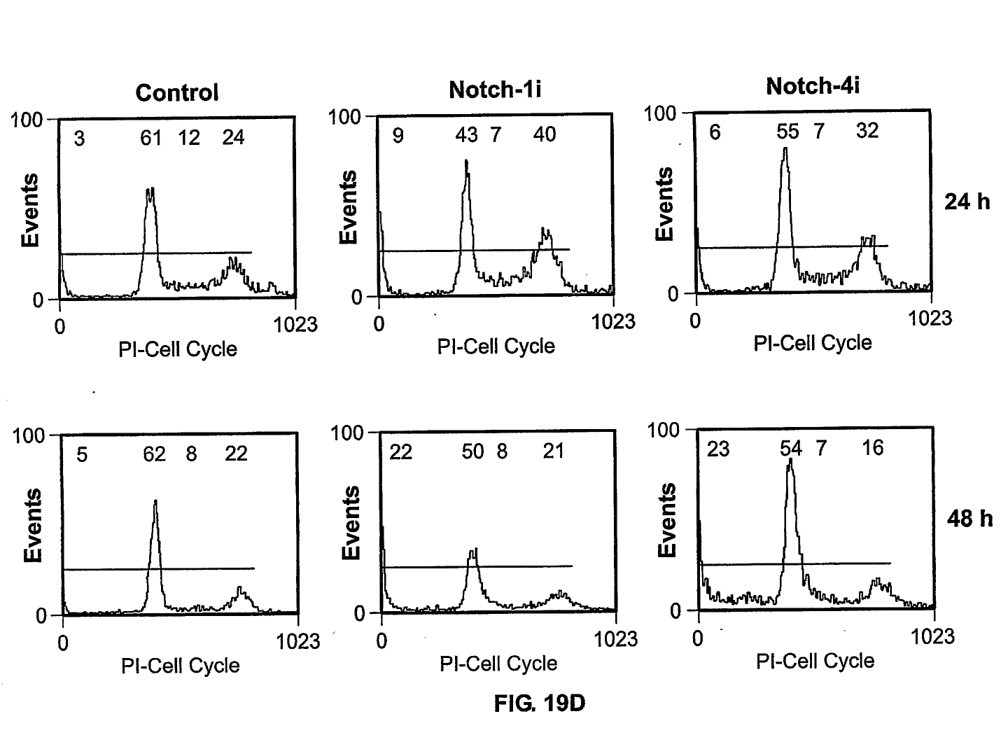

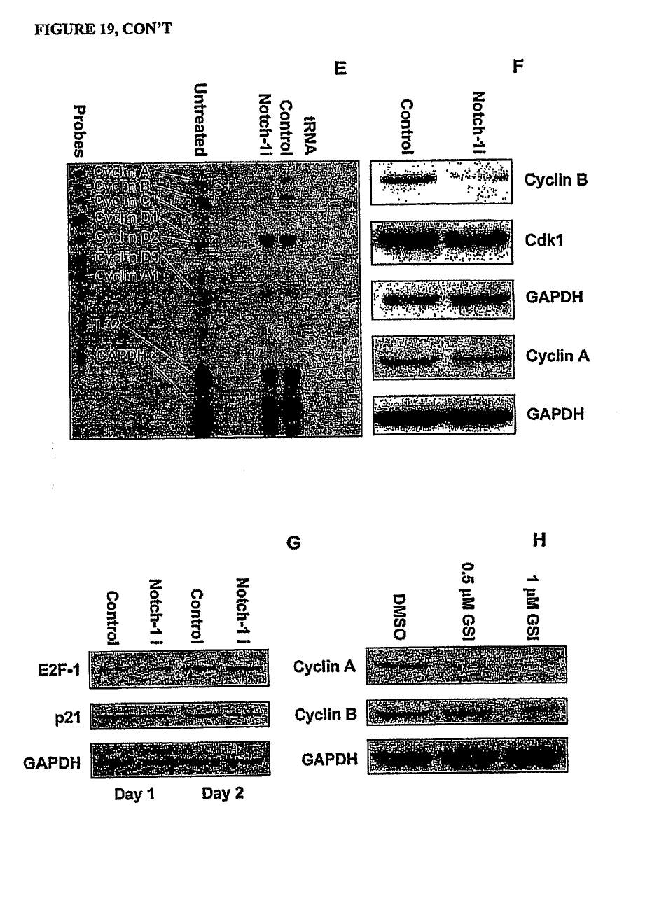

Both z-Leu-Leu-Nle-CHO (FIG. 19A) and RNAi silencing of Notch-1 (FIG. 19B) or Notch-4 (FIG. 19C) caused accumulation of cells in the G2/M stage of the cell cycle and a corresponding decrease in the number of cells in G1 and S. FIG. 19D shows representative raw data from flow cytometry experiments with the siRNA-treated cells. Numbers above the graphs indicate percentages of cells in subG1, G1, S and G2/M, respectively. With both Notch-1 siRNA- and Notch-4 siRNA-treated cells, the accumulation of cells in G2/M was maximal at 24 hours. At 48 hours, the effect disappeared and was replaced by an increased fraction of cells in the “subG1” region. The accumulation of cells in subG1 is indicative of cell death. These observations indicate a previously unknown effect of Notch signaling on the G2/M stages of the cell cycle in breast cancer cells.

To explore the effects on Notch-1 siRNA on the expression of cyclins, the main regulators of cell cycle progression, ribonuclease protection assays and Western blots on Notch-1 siRNA-treated cells were performed. Transfection of the MBA-MD231 cells with Notch-1 siRNA (5′-AAG TGT CTG AGG CCA GCA AGA-3′) (SEQ ID NO. 1) or control siRNA (5′-AAC AGT CGC GTT TGC GAC TGG-3′) (SEQ ID NO: 5), was performed as described above. Ribonuclease protection assays were performed by first preparing total RNA using a RNeasy Mini kit (QIAGEN Cat. #74104) according to the manufacturer's instructions. The multi-probe template set hCYC-1 (containing DNA templates for cyclin A, cyclin B, cyclin C, cyclin D1, cyclin D2, cyclin D3, cyclin A1 L32 and GAPDH) was purchased from BD Biosciences (Cat. # 556189). The DNA template was used to synthesize a [³²P]UTP (10 mCi/ml, Amersham Bioscience) labelled probe in the presence of a GACU pool using a T& RNA-polymerase (BD Bioscience RiboQuant RPA starter package, Cat. # 556144). Hybridization with 20 μg of each target RNA was performed at 56° C. overnight, followed by digestion with RNAse A and T1 according to the BD Bioscience standard protocol. After proteinase K treatment, samples were precipitated, loaded on a 4.75% acrylamide-urea gel, and run at 55 W with 0.5×TBE. Gels were dried in the gel dryer under vacuum for 2 h at 90° C. Then gels were exposed on Kodak film (Eastman Kodak, Rochester, N.Y.) with intensifying screens and developed at −70° C. Western blots were performed as described above. As shown in FIG. 19E, down regulation of Notch-1 decreases the expression of mRNAs for cyclins B1 and A, but not C, D, or A1. Western blot data confirmed these findings. FIG. 19F shows that siRNA silencing of Notch-1 causes a striking decrease in the steady state levels of cyclin B1 at 48 hours. Cyclin A was also decreased, with a maximal effect at 24 hours. CDK1 levels were only modestly affected. Cyclin A/cdk2 phosphorylates E2F-1/DP complexes at the end of S-phase, inactivating them. This prevents a re-initiation of DNA replication and assures that the genome is replicated only once at each S phase. Failure of cyclin A/cdk2 to inactivate E2F results in inappropriately persistent E2F activity, which may lead to apoptosis. E2F-1 is known to upregulate its own transcription. Consistent with this notion, Notch-1 siRNA caused a delayed (48 hours) accumulation of E2F-1 (FIG. 19G). A known Notch-1 target, p21, was downregulated at 24 hours (FIG. 19G). Additional experiments indicated that the ratio between nuclear and cytoplasmic levels of cyclin B1 is not affected by Notch silencing (not shown). z-Leu-Leu-Nle-CHO treatment (0.5 or 1 μM, 24 h) had identical effects as Notch-1 siRNA on cyclin A and B I levels (FIG. 19H).

### Example 19

### Notch-1 Inhibition Potentiates Paclitaxel-Induced Inhibition Breast Cancer Cell Growth

Three different types of breast cancer cells were contacted with varying concentrations of z-Leu-Leu-Nle-CHO (0-0.8 μM) and the mitotic poison Paclitaxel (0.01-1 μM). MDA-MB231 cells were obtained from ATCC (Rockville, Md.). T47D:C42 and T47D:A18 cells were a kind gift of Debra Tonetti (University of Illinois-Chicago). MDA-MB231 cells were cultured in DMEM medium with 10% FBS. T47D:C42 and T47D:A18 cells were propagated in RPMI 1640 with 10% FBS, 100 μM non-essential amino acid and 6 ng/ml insulin. Gamma-secretase inhibitor z-Leu-Leu-Nle-CHO (Calbiochem 565750) was dissolved in DMSO, aliquoted and stored at −80° C. Paclitaxel (Sigma, St. Louis Mo.) was dissolved in DMSO, aliquoted and stored at −80° C.

To determine whether z-Leu-Leu-Nle-CHO treatment potentiates the effect of Paclitaxel on human breast cancer cells, MDA-MB231, T47D:C42 and T47D:A18 cells were treated for 48 h with 0-0.8 μM z-Leu-Leu-Nle-CHO and various concentrations of Paclitaxel (0.008-1 μM) Following the z-Leu-Leu-Nle-CHO and Paclitaxel treatment, the cells were tested for growth inhibition as described above in Example 13.

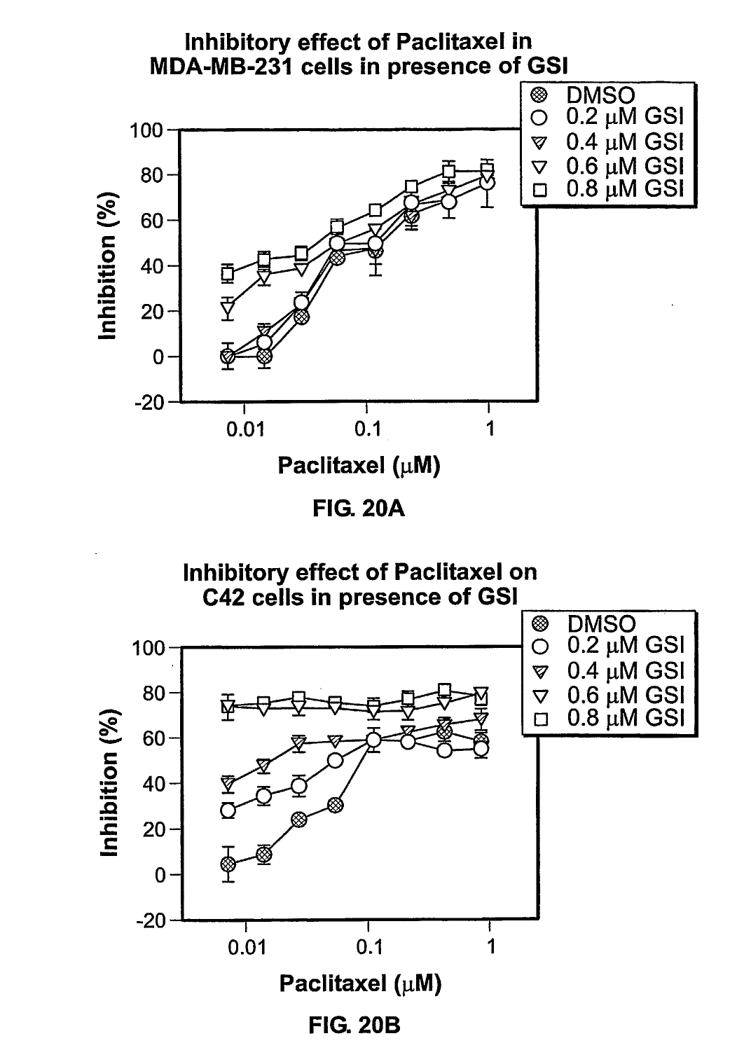

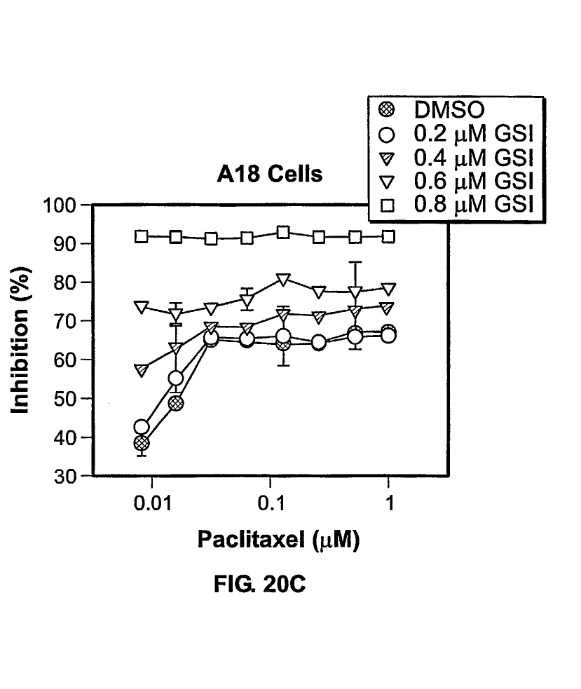

FIGS. 20A, 20B, and 20C show the percent inhibition of growth of MDA-MB-231, T47D:C42, or T47D:A18 breast cancer cell growth, respectively, as a function of Paclitaxel concentration in combination with different concentrations of z-Leu-Leu-Nle-CHO. All three different types of breast cancer cells contacted with varying concentrations of z-Leu-Leu-Nle-CHO and the mitotic poison Paclitaxel exhibited increased inhibition of cell growth. The results suggest that z-Leu-Leu-Nle-CHO and Paclitaxel act synergistically in inhibiting cell growth.

Each publication cited above or listed herein below is incorporated by reference in its entirety.

It should be understood that the foregoing disclosure emphasizes certain specific embodiments of the invention and that all modifications or alternatives equivalent thereto are within the spirit and scope of the invention as set forth in the appended claims.

What is claimed is:

1-32. (canceled)

33. A method for screening compounds for NOXA gene expression-inducing ability in cancer cells comprising:
(a) contacting cancer cells with a culture media in the presence and absence of a test compound;
(b) assaying the cells of step (a) for NOXA gene expression;
(c) comparing NOXA gene expression assayed in step (b) from cells contacted with culture media in the presence of the test compound with NOXA gene expression from cells contacted with culture media in the absence of the test compound; and
(d) identifying a compound that induces NOXA gene expression when NOXA gene expression is higher in cells contacted in the presence of the test compound than in cells contacted in the absence of the test compound.

34. The method of claim 33, further comprising:
(e) assaying NOXA gene expression-inducing compounds identified in step (d) for the ability to induce apoptosis in cancer cells.

35. The method of claim 33, further comprising:
(f) assaying NOXA gene expression-inducing compounds identified in step (d) for the ability to inhibit cell growth in cancer cells.

36. A method for screening a test compound for Notch-1 gene expression or activity inhibiting ability in cancer cells, the method comprising the steps of:
(a) contacting cancer cells with a culture media in the presence and absence of a test compound;
(b) assaying the cells of step (a) for Notch-1 gene expression or activity;
(c) comparing Notch-1 gene expression or activity assayed in step (b) from cells contacted with culture media in the presence of the test compound with Notch-1 gene expression or activity from cells contacted with culture media in the absence of the test compound; and
(d) identifying a compound that inhibits Notch-1 gene expression or activity when Notch-1 gene expression or activity is lower in cells contacted in the presence of the test compound than in cells contacted in the absence of the test compound.

37. The method of claim 36, further comprising:
(e) assaying Notch-1 gene expression or activity-inhibiting compounds identified in step (d) for the ability to induce apoptosis in cancer cells.

38. The method of claim 36, further comprising:
(e) assaying Notch-1 gene expression or activity-inhibiting compounds identified in step (d) for the ability to inhibit cell growth in cancer cells.

39. The method of claim 33 or 36, wherein the cancer cells are deficient in p53 tumor suppressor protein.

40. The method of claims 33 or 36, wherein the cancer cells are melanoma cells, myeloma cells, prostate cancer cells, osteosarcoma cells, cervical cancer cells or breast cancer cells.

41-82. (canceled)


SEQUENCE LISTING (6 sequences)

SEQ ID NO: 1 - DNA, 21 bp, Artificial
  Synthetic; Notch-1 siRNA
aagtgtctgaggccagcaaga

SEQ ID NO: 2 - DNA, 20 bp, Artificial
  Synthetic; NOXA targeted sequence
tcagtctactgatttactgg

SEQ ID NO: 3 - DNA, 20 bp, Artificial
  Synthetic; Control Oligonucleotide
ccttccctgaaggttcctcc

SEQ ID NO: 4 - DNA, 20 bp, Artificial
  Synthetic; Universal Scrambled Control Oligonucleotide
ttctacctcgcgcgatttac

SEQ ID NO: 5 - DNA, 21 bp, Artificial
  Synthetic; Control siRNA
aacagtcgcgtttgcgactgg

SEQ ID NO: 6 - DNA, 21 bp, Artificial
  Synthetic; Notch-4 siRNA
aaccctgtgccaatggaggca